(19) United States
(12) Patent Application Publication
(10) Pub. No.: US 2023/0038929 A1
(43) Pub. Date: Feb. 9, 2023

(54) Fused Tricyclic Heterocyclic Compounds and Uses Thereof

(71) Applicant: Verge Analytics, Inc., South San Francisco, CA (US)

(72) Inventors: Mark D. Rosen, South San Francisco, CA (US); Robert A. Galemmo, JR., San Francisco, CA (US); William J. Guilford, South San Francisco, CA (US); Weiling Liang, South San Francisco, CA (US); Irene Y. Choi, Pacifica, CA (US); Bryan Kopek, South San Francisco, CA (US); Jane Rhodes, South San Francisco, CA (US)

(73) Assignee: Verge Analytics, Inc., South San Francisco, CA (US)

(21) Appl. No.: 17/782,444

(22) PCT Filed: Dec. 4, 2020
(86) PCT No.: PCT/US2020/063298

§ 371 Date: Jun. 3, 2022

Related U.S. Application Data
(60) Provisional application No. 62/944,222, filed on Dec. 5, 2019.
(60) Provisional application No. 63/036,228, filed on Jun. 8, 2020.

(51) Int. Cl. C07D 491/147

(52) U.S. Cl. CPC C07D 491/147

(57) ABSTRACT

The present disclosure provides compounds that are inhibitors of PIKfyve and/or PI3 kinases, and are therefore useful for the treatment of neurological diseases that are treatable by inhibition of PIKfyve. Also provided are pharmaceutical compositions containing such compounds, and methods of treatment using such compounds.

### CROSS-REFERENCE TO RELATED APPLICATIONS

This application claims priority to U.S. Provisional Application No. 62/944,222, filed on Dec. 5, 2019, and U.S. Provisional Application No. 63/036,228, filed on Jun. 8, 2020, the disclosures of each of which are hereby incorporated by reference in their entireties.

### TECHNICAL FIELD

The present disclosure provides compounds that are phosphoinositide kinase inhibitors, in particular FYVE-type finger-containing phosphoinositide kinase (“PIKfyve”) inhibitors and are therefore useful for the treatment of central nervous system diseases. Also provided are pharmaceutical compositions containing such compounds and processes for preparing such compounds.

### BACKGROUND

Phosphoinositide kinases (PIKs) catalyze the phosphorylation of phosphatidylinositol, which is a component of eukaryotic cell membranes, and related phospholipids called phosphoinositides. The phosphoinositides are involved in the regulation of diverse cellular processes, including cellular proliferation, survival, cytoskeletal organization, vesicle trafficking, glucose transport, and platelet function. Fruman et al., “Phosphoinositide Kinases,” Ann. Review. Biochem. 1998, 67, 481-507. Phosphorylated derivatives of phosphatidylinositol regulate cytoskeletal functions, membrane trafficking, and receptor signaling by recruiting protein complexes to cell and endosomal membranes.

FYVE-type finger-containing phosphoinositide kinase (PIKfyve; also known as phosphatidylinositol-3-phosphate 5-kinase type III or PIPKIII) is a ubiquitously expressed PIK with both lipid and protein kinase activity. In its capacity as a lipid kinase, the enzyme phosphorylates the D-5 position in endosomal phosphatidylinositol and phosphatidylinositol-3-phosphate (PI3P) to generate the corresponding 5-phosphate phospholipid analogs. Shisheva et al., Cell Biol. Int. 2008, 32(6), 591. PI3P is found in cell membranes with roles in protein trafficking, protein degradation, and autophagy. Nascimbeni et al., FEBS J. 2017, 284, 1267-1278. PIKfyve regulates endomembrane homeostasis and plays a role in the biogenesis of endosome carrier vesicles from early endosomes. The enlarged endosome/lysosome structure was observed in cells expressing PIKfyve dominant negative or siRNA. Ikonomov et al., J. Biol. Chem. 2001, 276(28), 26141-26147; Rutherford et al., J. Cell Sci. 2006, 119, 3944-3957. Inhibition of PIKfyve activity increases levels of PI3P, stimulating autophagy and improving motor neuron health. Phosphorylated inositides produced by PIKfyve are localized in various cellular membranes and organelles, consistent with the various PIKfyve functions of endolysosomal transport, endomembrane homeostasis, and biogenesis of endosome carrier vesicles (ECV)/multivesicular bodies (MVB) from early endosomes. Further, PIKfyve is required for endocytic-vacuolar pathway and nuclear migration. Thus, PIKfyve helps maintain proper morphology of the endosome and lysosome.

In mammalian cells, PI3P levels are regulated by the reciprocal activities of PIKfyve and the phosphatidylinositol 3,5-bisphosphate 5-phosphatase (FIG4). Zolov et al., “In vivo, PIKfyve generates PI(3,5)P2, which serves as both a signaling lipid and the major precursor for PI5P,” Proc. Natl. Acad. Sci. USA 2012, 109(43), 17472-17477. Normally, FIG4 is localized on the cytoplasmic surface of endolysosomal vesicles in a complex. Inhibition of PIKfyve would mimic overexpression of FIG4, thereby increasing levels of PI3P, stimulating autophagy, and improving motor neuron health. Numerous diseases are correlated with FIG4 deficiencies, such as deleterious FIG4 mutations or diminished FIG4 function, and are therefore suitable as target diseases for treatment with PIKfyve inhibitors, including amyotrophic lateral sclerosis (ALS), primary lateral sclerosis (PLS), Charcot-Marie-Tooth (including type 4J (CMT4J)), and Yunis-Varon syndrome.

Exemplary diseases associated with FIG4 deficiencies are amyotrophic lateral sclerosis (ALS), primary lateral sclerosis (PLS), Charcot-Marie-Tooth (including type 4J (CMT4J)), Yunis-Varon syndrome, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, fronto-temporal dementia, diseases of neuronal nuclear inclusions of polyglutamine and intranuclear inclusion bodies, disease of Marinesco and Hirano bodies, Alzheimer's disease, neurodegeneration, spongiform neurodegeneration, autophagy, peripheral neuropathy, leukoencephalopathy, motor neuropathy, sensory neuropathy, Bharadwaj et al., Hum. Mol. Genet. 2016, 25(4), 682-692.

PIKfyve inhibitors are useful in a range of neurological disorders, such as tauopathies (including but not limited to Alzheimer's disease, progressive supranuclear palsy, corticobasal syndrome, frontotemporal dementias, and chronic traumatic encephalopathy), traumatic brain injury (TBI), cerebral ischemia, ALS, fronto-temporal dementia (FTD), Guillain-Barré Syndrome, chronic inflammatory demyelinating polyneuropathy, multiple sclerosis, CMT, lysosomal storage diseases (including but not limited to Fabry's disorder, Gaucher's disorder, Niemann Pick C, Tay-Sachs, and Mucolipidosis type IV), as well as several types of neuropathies. Other therapeutic targets for intervention with PIKfyve inhibitors include Huntington's disease and psychiatric disorders (such as ADHD, schizophrenia, mood disorders including but not limited to major depressive disorder, bipolar disorder I, and bipolar disorder II). Gardiner et al., “Prevalence of carriers of intermediate and pathological polyglutamine disease-associated alleles among large population-based cohorts,” JAMA Neural. 2019, 76(6), 650-656; PCT Publ. No. WO2016/210372; US Publ. No. US2018/0161335.

In some aspects, the compounds described herein inhibit PI3K, including various isoforms of PI3K such as PI3Kα, β, δ, and/or γ. PI3K, also known as phosphoinositide 3-kinase or phosphatidylinositol 3-kinase, is a family of enzymes involved in cellular functions such as cell growth, proliferation, differentiation, motility, survival, and intracellular trafficking. PI3K inhibitors are useful as potential therapeutics in a range of disease states including, for example, central nervous system diseases

### SUMMARY

In a first aspect, this disclosure is directed to a compound of Formula (I):

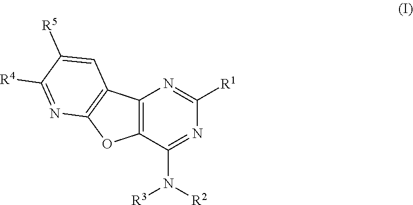

wherein:
R² is H, C₁₋₄alkyl, or —NR^{a}R^{b};
   wherein R^{a} is H or C₁₋₄alkyl; and
   R^{b} is −L—R^{c};
   wherein L is a bond, —C(O)—, —C(O)O—, or —C₁₋₄alkylene-; and
   R^{c} RC is phenyl, monocyclic cycloalkyl, monocyclic heterocycloalkyl, or monocyclic heteroaryl, wherein each phenyl, cycloalkyl, heterocycloalkyl, or heteroaryl is optionally substituted with one, two, or three R^{d} substituents;
      wherein each R^{d} substituent is independently C₁₋₄alkyl, C₁₋₄alkenyl, C₁₋₄alkynyl, —O—C₁₋₄alkyl, halo, cyano, nitro, azido, halo—C₁₋₄alkyl, —O—C₁₋₄-haloalkyl, —NR^{g}C(═O)R^{h}, —NR^{g}C(═O)NR^{g}R^{h}, —NR^{g}C(═O)OR^{h}, =NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —O^{g}, —SR^{g}, —S(═O)R^{g}, S(═O)₂R^{g}, —OS(═O)₁₋₂R^{g}, —S(═O)₁₋₂OR^{g}, —S(═O)₁₋₂NR^{g}R^{h}, phenyl, —C₁₋₄alkyl-phenyl, monocyclic cycloalkyl, —C₁₋₄alkyl-cycloalkyl, monocyclic heterocycloalkyl, or monocyclic heteroaryl;
      wherein each phenyl, monocyclic cycloalkyl, monocyclic heterocycloalkyl, or monocyclic heteroaryl of R^{d} is optionally substituted with one, two, or three substituents R^{e};
      wherein each R^{e} substituent is independently C₁₋₄alkyl, C₁₋₄alkenyl, C₁₋₄alkynyl, halo, cyano, nitro, azido, —OH, halo—C₁₋₄alkyl, —O—C₁₋₄alkyl, or —O—C₁₋₄-haloalkyl;
   R^{g} and R^{h} are each independently H or C₁₋₄alkyl;
R² and R³ taken together with the nitrogen to which they are attached form a monocyclic heterocycloalkyl, optionally substituted with one, two, or three substituents;
   wherein each R^{j} substituent is independently C₁₋₄alkyl, —OH, —NR^{k}R^{l}, halo, halo—C₁₋₄alkyl, -O—C₁₋₄alkyl, or —O—C₁₋₄-haloalkyl; where R^{k} and R^{l} are each independently H or C₁₋₄alkyl; and
R⁴ and R⁵ are each independently H, C₁₋₄alkyl, halo, —OH, or —OC₁₋₄alkyl, wherein each alkyl is optionally substituted with —NR^{m}Rⁿ;
   wherein R^{o} and Rⁿ are each independently H or C₁₋₄alkyl, or R^{m} and Rⁿ taken together with the nitrogen to which they are attached form a monocyclic heterocycloalkyl, optionally substituted with one or two R^{o} substituents;
      wherein each R^{o} substituent independently C₁₋₄alkyl, —OH, —OC₁₋₄alkyl, halo, cyano, or —NR^{p}R^{q};
         wherein R′ and Rq are each independently H or C₁₋₄alkyl; or a pharmaceutically acceptable salt thereof.

In a second aspect, this disclosure is directed to a pharmaceutical composition comprising a compound of Formula (I) (or any of the embodiments thereof described herein), or a pharmaceutically acceptable salt thereof, and a pharmaceutically acceptable excipient.

In a third aspect, this disclosure is directed to a method of inhibiting PIKfyve and/or a PI3 kinase in a subject in need thereof comprising administering to the subject an effective amount of a compound of Formula (I) (or any of the embodiments thereof described herein), or a pharmaceutically acceptable salt thereof, or a pharmaceutical composition comprising a compound of Formula (I) (or any of the embodiments thereof described herein), or a pharmaceutically acceptable salt thereof.

In a fourth aspect, this disclosure is directed to a method of treating a disease treatable by inhibition of PIKfyve and/or a PI3 kinase activity in a subject in need thereof comprising administering to the subject in need thereof a compound of Formula (I) (or any of the embodiments thereof described herein), or a pharmaceutically acceptable salt thereof, or a pharmaceutical composition comprising a compound of Formula (I) (or any of the embodiments thereof described herein), or a pharmaceutically acceptable salt thereof.

In a fourth aspect, the disclosure is directed a compound of Formula (I) (and any embodiments thereof described herein), or a pharmaceutically acceptable salt thereof, for use as a medicament. In one embodiment, the use of the compound of Formula (I) and/or a pharmaceutically acceptable salt thereof is for treating a disease treatable by inhibition of PIKfyve and/or a PI3 kinase or associated with PIKfyve and/or PI3 kinase activity.

In a fifth aspect is the use of a compound of Formula (I) (or any of the embodiments thereof described herein), or a pharmaceutically acceptable salt thereof, in the manufacture of a medicament for treating a disease in a mammal in which PIKfyve or PI3K contributes to the pathology and/or symptoms of the disease.

### DETAILED DESCRIPTION

Unless otherwise stated, the following terms used in the specification and claims are defined for the purposes of this disclosure and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylsulfonyl” means a —SO₂R radical where R is alkyl as defined above, e.g., methylsulfonyl, ethylsulfonyl, and the like.

“Amino” means a —NH₂.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with an alkoxy group, (in one embodiment one or two alkoxy groups), as defined above, e.g., 2-methoxyethyl, 1-, 2-, or 3-methoxypropyl, 2-ethoxyethyl, and the like.

“Alkoxycarbonyl” means a —C(O)OR radical where R is alkyl as defined above, e.g., methoxycarbonyl, ethoxycarbonyl, and the like.

“Acyl” means a —COR radical where R is alkyl, haloalkyl, or cycloalkyl, e.g., acetyl, propionyl, cyclopropylcarbonyl, and the like. When R is alkyl, the radical is also referred to herein as alkylcarbonyl.

“Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Carboxy” means —COOH.

“Halo” means fluoro, chloro, bromo, or iodo; in one embodiment fluoro or chloro.

“Haloalkyl” means alkyl radical as defined above, which is substituted with one or one to five halogen atoms (in one embodiment fluorine or chlorine,) including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this disclosure as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it can be referred to in this disclosure as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl. Further examples include, but are not limited to, 2-hydroxyethyl, 2,3-dihydroxypropyl, and 1-(hydroxymethyl)-2-hydroxyethyl.

“Heterocyclyl” means a saturated or unsaturated monovalent monocyclic or bi-cyclic group (fused bi-cyclic or bridged bi-cyclic) of 4 to 10 ring atoms in which one or two ring atoms are heteroatom selected from N, O, and S(O)n, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, oxetanyl, pyrrolidino, piperidino, homopiperidino, 2-oxopyrrolidinyl, 2-oxopiperidinyl, morpholino, piperazino, tetrahydropyranyl, thiomorpholino, hexahydropyrrolo[1,2-a]pyrazin-6(2H)-one-yl, tetrahydro-1H-oxazolo[3,4-a]pyrazin-3(5H)-one-yl, 5,6,7,8-tetrahydro-[1,2,4]triazolo[4,3-a]pyrazine-yl, 3-oxa-8-azabicyclo[3.2.1]octane-yl, and the like. When the heterocyclyl ring is unsaturated it can contain one or two ring double bonds provided that the ring is not aromatic.

“Heterocyclylalkyl” means a -(alkylene)-R radical where R is heterocyclyl ring as defined above e.g., tetraydrofuranylmethyl, piperazinylmethyl, morpholinylethyl, and the like.

“Heterocycloamino” means a saturated or unsaturated monovalent monocyclic group of 4 to 8 ring atoms in which one or two ring atoms are heteroatom selected from N, O, or S(O)n, where n is an integer from 0 to 2, the remaining ring atoms being C provided that at least one of the ring atoms is N. Additionally, one or two ring carbon atoms in the heterocycloamino ring can optionally be replaced by a —CO— group. When the heterocycloamino ring is unsaturated it can contain one or two ring double bonds provided that the ring is not aromatic.

“Heterocycloaminoalkyl” means a —(alkylene)-R radical where R is heterocycloamino as described above.

“Heteroaryl” means a monovalent monocyclic or bicyclic aromatic radical of 5 to 10 ring atoms where one or more, (in one embodiment one, two, or three), ring atoms are heteroatom selected from N, 0, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Mammal” as used herein means domesticated animals (such as dogs, cats, and horses), and humans. In one embodiment, mammal is a human.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Oxo” means an =(O) group and “carbonyl” means a >C(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

A “therapeutically effective amount” means the amount of a compound of Formula (I) (or any of the embodiments thereof described herein), that, when administered to a mammal for treating a disease, is sufficient to treat the disease. The “therapeutically effective amount” will vary depending on the compound, the disease and its severity and the age, weight, etc., of the mammal to be treated.

The compounds described herein may in some cases exist as diastereomers, enantiomers, or other stereoisomeric forms. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Certain compounds of Formula (I) (or any of the embodiments thereof described herein) and/or a pharmaceutically acceptable salt thereof can exist as tautomers and/or geometric isomers. All possible tautomers and cis and trans isomers, as individual forms and mixtures thereof, are within the scope of this disclosure. For example, pyrazole tautomers as shown below are equivalent structures. The depiction of one such structure is intended to encompass both structures.

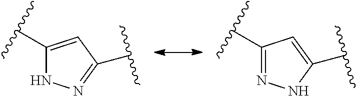 [SMILES: *c1cc(*)[nH]n1 ; *c1cc(*)[nH]n1]

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

Pharmaceutically acceptable salts of the compounds of Formula (I) (or any of the embodiments thereof described herein) are within the scope of this disclosure. In addition, the compounds described herein include hydrates and solvates of the compounds or pharmaceutically acceptable salts thereof.

The present disclosure also includes the prodrugs of compounds of Formula (I) (or any of the embodiments thereof described herein) and/or a pharmaceutically acceptable salt thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt thereof are also within the scope of this disclosure.

The present disclosure also includes polymorphic forms (amorphous as well as crystalline) and deuterated forms of compounds of Formula (I) (or any of the embodiments thereof described herein) and/or a pharmaceutically acceptable salt thereof.

The compounds disclosed herein, in some embodiments, are used in different enriched isotopic forms, e.g., enriched in the content of ²H, ³H, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In one aspect is a compound of Formula (I):

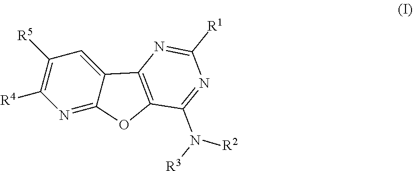

wherein:
R¹ is H, C₁₋₄alkyl, or —NR^{a}R^{b};
   wherein R^{a} is H or C₁₋₄alkyl; and
   R^{b} is —L−R^{c};
   wherein L is a bond, —C(O)—, —C(O)O—, or —C₁₋₄alkylene-; and
   R^{c} is phenyl, monocyclic cycloalkyl, monocyclic heterocycloalkyl, or monocyclic heteroaryl, wherein each phenyl, cycloalkyl, heterocycloalkyl, or heteroaryl is optionally substituted with one, two, or three R^{d} substituents;
      wherein each R^{d} substituent is independently C₁₋₄alkyl, C₁₋₄alkenyl, C₁₋₄alkynyl, —O—C₁₋₄alkyl, halo, cyano, nitro, azido, halo—C₁₋₄alkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, =NOR_{g}, —NR^{g}S(═O)₁₋₂R^{h}, —NR^{g}S(═O)₁₋₂NR^{g}R^{h}, =NSO2R_{g}, —C(═O)R_{g}, —C(═O)OR_{g}, —OC(═O)OR_{g}, —OC(═O)R_{g}, —C(═O)NR^{g}R^{h}, —OC(═O)NR^{g}R^{h}, —OR_{g}, —SR_{g}, —S(═O)R_{g}, —S(═O)₂R_{g}, —OS(═O)₁₋₂R_{g}, —S(═O)₁₋₂OR_{g}, —S(═O)₁₋₂NR^{g}R^{h}, phenyl, —C₁₋₄alkyl-phenyl, monocyclic cycloalkyl, —C₁₋₄alkyl-cycloalkyl, monocyclic heterocycloalkyl, or monocyclic heteroaryl;
      wherein each phenyl, monocyclic cycloalkyl, monocyclic heterocycloalkyl, or monocyclic heteroaryl of R^{d} is optionally substituted with one, two, or three substituents R^{e};
         wherein each R^{e} substituent is independently C₁₋₄alkyl, C₁₋₄alkenyl, C₁₋₄alkynyl, halo, cyano, nitro, azido, —OH, halo—C₁₋₄alkyl, —O—C₁₋₄alkyl, or —O—C₁₋₄haloalkyl;
      R^{g} and R^{h} are each independently H or C₁₋₄alkyl;
R² and R³ taken together with the nitrogen to which they are attached form a monocyclic heterocycloalkyl, optionally substituted with one, two, or three R¹ substituents;
   wherein each R¹ substituent is independently C₁₋₄alkyl, —OH, —NR^{k}R^{l}, halo, halo—C₁₋₄alkyl, —O—C₁₋₄alkyl, or —O—C₁₋₄-haloalkyl; where R^{k} and R^{I} are each independently H or C₁₋₄alkyl; and
R⁴ and R⁵ are each independently H, C₁₋₄alkyl, halo, —OH, or —OC₁₋₄alkyl, wherein each alkyl is optionally substituted with —NR^{m}Rⁿ;
   wherein R^{m} and R⁵ are each independently H or C₁₋₄alkyl, or R^{m} and Rⁿ taken together with the nitrogen to which they are attached form a monocyclic heterocycloalkyl, optionally substituted with one or two R^{o} substituents;
      wherein each R^{o} substituent independently C₁₋₄alkyl, —OH, —OC₁₋₄alkyl, halo, cyano, or —NRₚR^{q};
      wherein R^{p} and R^{q} are each independently H or C₁₋₄alkyl;
         or a pharmaceutically acceptable salt thereof.

In some embodiments, R¹ is —NR^{a}R^{b}. In some embodiments, R¹ is H. In some embodiments, R¹ is C₁₋₄alkyl. In some embodiments, R¹ is methyl.

In some embodiments, R^{a} is H. In some embodiments, R^{a} is C₁₋₄alkyl. In some embodiments, R^{a} is methyl.

In some embodiments, L is a bond. In some embodiments, L is —C(O)— or —C(O)O—. In some embodiments, L is —C₁₋₄alkylene-. In some embodiments, L is methylene or ethylene.

In some embodiments, R^{c} is optionally substituted phenyl. In some embodiments, R^{c} is optionally substituted monocyclic cycloalkyl. In some embodiments, R^{c} is optionally substituted cyclopropyl, cyclobutyl, cyclopentyl, or cyclohexyl. In some embodiments, R^{c} is optionally substituted cyclopropyl. In some embodiments, R^{c} is optionally substituted monocyclic heterocycloalkyl. In some embodiments, R^{c} is optionally substituted pyrrolidinyl, tetrahydrofuranyl, piperidinyl, morpholinyl, or piperazinyl. In some embodiments, R^{c} is optionally substituted monocyclic heteroaryl. In some embodiments, R^{c} is optionally substituted pyrrole, imidazole, pyrazole, triazole, tetrazole, furan, oxazole, isoxazole, thiazole, isothiazole, pyridine, pyrimidine, pyrazine, or pyridazine. In some embodiments, W is optionally substituted pyrazole, imidazole, pyridine, or pyrimidine. In some embodiments, R^{c} is optionally substituted pyrazole. In some embodiments, each R^{c} is optionally substituted with one or two R^{d} substituents.

In some embodiments, each R^{d} substituent is independently C₁₋₄alkyl, C₁₋₄alkenyl, C₁₋₄alkynyl, —O—C₁₋₄alkyl, halo, cyano, nitro, azido, halo—C₁₋₄alkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, =NOR_{g}, —NR^{g}S(═O)₁₋₂R^{h}, —NR^{g}S(═O)₁₋₂NR^{g}R^{h}, ═NSO₂R_{g}, —C(═O)R_{g}, —C(═O)OR_{g}, —OC(═O)OR_{g}, —OC(═O)R_{g}, —C(═O)NR^{g}R^{h}, —OC(═O)NR^{g}R^{h}, —OR_{g}, —SR_{g}, —S(═O)R_{g}, —S(═O)₂R_{g}, —OS(═O)₁₋₂R_{g}, —S(═O)₁-₂₀R_{g}, —S(═O)₁₋₂NR^{g}R^{h}, phenyl, —C₁₋₄alkyl-phenyl, monocyclic cycloalkyl, —C₁₋₄alkyl-cycloalkyl, monocyclic heterocycloalkyl, or monocyclic heteroaryl, wherein the phenyl, monocyclic cycloalkyl, monocyclic heterocycloalkyl, and monocyclic heteroaryl of R^{d} are each optionally substituted with one or two substituents R^{e}. In some embodiments, each R^{d} substituent is independently C₁₋₄alkyl, halo—C₁₋₄alkyl, phenyl, —C₁₋₄alkyl-phenyl, pyridyl, thiophenyl, cycloalkyl, or —C₁₋₄alkyl-cycloalkyl, wherein the phenyl, pyridyl, and thiophenyl are each optionally substituted with one or two substituents R^{e}. In some embodiments, each R^{d} substituent is independently methyl, ethyl isopropyl, —CF₃, —OCH₃, —OCF₃, phenyl, pyridyl, thiophenyl, benzyl, cyclopropyl, cyclobutyl, cyclopentyl, cyclopropylmethyl, cyclobutylmethyl, or cyclopentylmethyl, wherein the phenyl, cycloalkyl, and heteroaryl of R^{d} are each optionally substituted with one or two substituents R^{e}.

In some embodiments, each R^{e} substituent is independently C₁₋₄alkyl, halo, halo-C₁₋₄alkyl, —O—C₁₋₄alkyl, or —O—C₁₋₄-haloalkyl. In some embodiments, each R^{e} substituent is independently methyl, —CF₃, fluoro, chloro, —OCH₃, or —OCF₃. In some embodiments, each R^{d} substituent is independently methyl, ethyl isopropyl, —CF₃, phenyl, pyridyl, thiophenyl, benzyl, cyclopropyl, cyclobutyl, cyclopentyl, cyclopropylmethyl, cyclobutylmethyl, or cyclopentylmethyl, wherein each R^{e} is independently methyl, —CF₃, fluoro, chloro, —OCH₃, or —OCF₃.

In some embodiments, R^{g} and R^{h} are each independently H or methyl.

In some embodiments, R² and R³ taken together with the nitrogen to which they are attached form pyrrolidine, piperidine, piperazine, morpholine, thiomorpholine, or thiomorpholine-1,1-dioxide, each optionally substituted with one, two, or three R^{j} substituents. In some embodiments, R² and R³ taken together with the nitrogen to which they are attached form morpholine, optionally substituted with one or two R^{j} substituents.

In some embodiments, each substituent is independently methyl, hydroxy, —OCH₃, halo, —CF₃, or —OCF₃.

In some embodiments, R^{k} and R^{l} are each independently H or methyl.

In some embodiments, R⁴ and R⁵ are each H. In some embodiments, one of R⁴ and R⁵ is H and the other is C₁₋₄alkyl, halo, —OH, or —OC₁₋₄alkyl, wherein each alkyl is optionally substituted with —NR^{m}Rⁿ. In some embodiments, one of R⁴ and R⁵ is H and the other is -OH, halo, or —OCH₃. In some embodiments, one of R⁴ and R⁵ is H and the other is C₂₋₃alkyl substituted with —NR^{m}Rⁿ.

In some embodiments, R^{m} and Rⁿ are each independently H or C₁₋₄alkyl. In some embodiments, R^{m} and Rⁿ are each methyl. In some embodiments, R^{m} and Rⁿ taken together with the nitrogen to which they are attached form a monocyclic heterocycloalkyl, optionally substituted with one or two R^{o} substituents. In some embodiments, R^{m} and Rⁿ taken together with the nitrogen to which they are attached form pyrrolidine, piperidine, piperazine, morpholine, thiomorpholine, or thiomorpholine-1,1-dioxide, each optionally substituted with one or two R^{o} substituents. In some embodiments, R^{m} and Rⁿ taken together with the nitrogen to which they are attached form pyrrolidine, piperidine, piperazine, or morpholine, each optionally substituted with one or two R^{o} substituents.

In some embodiments, each R^{o} substituent is C₁₋₄alkyl. In some embodiments, R^{o} and R^{q} are each independently H or methyl.

In some embodiments, the compound of Formula (I) or the pharmaceutically acceptable salt thereof is a compound of Formula (II):

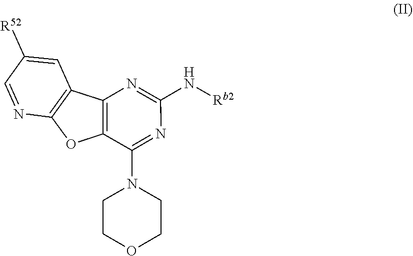

wherein
R^{b2} is pyrazole, phenyl, pyridyl, or pyrimidinyl, each optionally substituted with one or two substituents selected from C₁₋₄alkyl, phenyl, and pyridyl,
   wherein the phenyl and pyridyl substituents are optionally substituted with methyl, —CF₃, fluoro, chloro, —OCH₃, —OCF₃, or phenyl;
R⁵² is H or —C₂₋₃-alkylene-NR^{m2}Rⁿ²;
   wherein R^{m2} and Rⁿ² are each independently H or methyl, or R^{m2} and Rⁿ² taken together with the nitrogen to which they are attached form pyrrolidine, piperidine, piperazine, or morpholine, each optionally substituted with methyl;
or a pharmaceutically acceptable salt thereof.

In some embodiments, R^{b2} is pyrazole, optionally substituted with methyl, —CF₃, fluoro, chloro, —OCH₃, —OCF₃, or phenyl.

In some embodiments, the compound of Formula (I) or the pharmaceutically acceptable salt thereof is a compound of Formula (III):

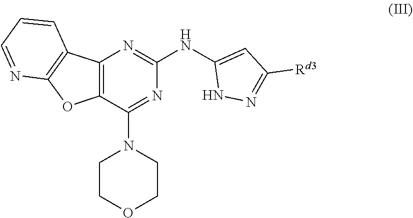

wherein
R^{d3} is (a) methyl, ethyl, isopropyl, —CF₃, —OCH₃, or —OCF₃, or (b) phenyl, benzyl, cyclopropyl, cyclobutyl, cyclopentyl, cyclopropylmethyl, cyclobutylmethyl, cyclopentylmethyl, thiophenyl, or pyridyl, wherein the phenyl, benzyl, each optionally substituted with one or two substituents independently selected from methyl, fluoro, chloro, —CF₃, —OCH₃, and —OCF₃;
or a pharmaceutically acceptable salt thereof.

In some embodiments, the compound is a compound selected from those of Table 1:

TABLE 1

| Compd | | |
|---|---|---|
| # | Chemical Name | Structure |
| 1 | N-(3-(4-Fluorophenyl)-1H-pyrazol- 5-yl)-4- morpholinopyrido[3′,2′:4,5]furo[3,2- d]pyrimidin-2-amine hydrochloride | 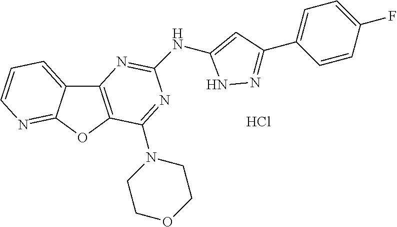 [SMILES: Fc1ccc(-c2cc(Nc3nc(N4CCOCC4)c4oc5ncccc5c4n3)[nH]n2)cc1 ; Cl] |
| 2 | N-Benzyl-4- morpholinopyrido[3′,2′:4,5]furo[3,2- d]pyrimidin-2-amine | 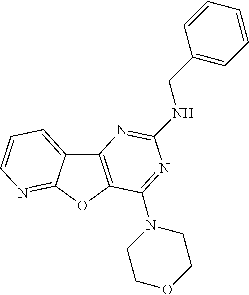 [SMILES: c1ccc(CNc2nc(N3CCOCC3)c3oc4ncccc4c3n2)cc1] |
| 3 | N-(4-Morpholinobenzofuro[3,2- d]pyrimidin-2-yl)benzamide | 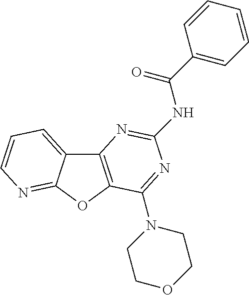 [SMILES: O=C(Nc1nc(N2CCOCC2)c2oc3ncccc3c2n1)c1ccccc1] |
| 4 | 7-(2-(Dimethylamino)ethoxy)-4- morpholino-N-(3-phenyl-1H- pyrazol-5- yl)pyrido[3′,2′:4,5]furo[3,2- d]pyrimidin-2-amine hydrochloride | 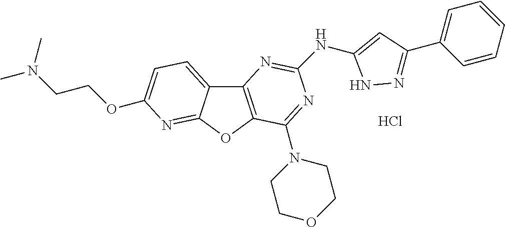 [SMILES: CN(C)CCOc1ccc2c(n1)oc1c(N3CCOCC3)nc(Nc3cc(-c4ccccc4)n[nH]3)nc12 ; Cl] |
| 5 | 4-Morpholino-2-((3-phenyl-1H- pyrazol-5- yl)amino)pyrido[3′,2′:4,5]furo[3,2- d]pyrimidin-7-ol hydrochloride | 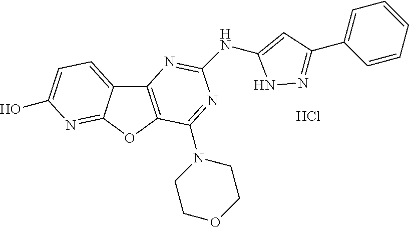 [SMILES: Oc1ccc2c(n1)oc1c(N3CCOCC3)nc(Nc3cc(-c4ccccc4)n[nH]3)nc12 ; Cl] |
| 6 | 4-Morpholino-N-(3-(pyridin-2-yl)- 1H-pyrazol-5- yl)pyrido[3′,2′:4,5]furo[3,2- d]pyrimidin-2-amine hydrochloride | 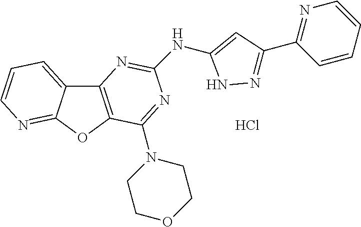 [SMILES: c1ccc(-c2cc(Nc3nc(N4CCOCC4)c4oc5ncccc5c4n3)[nH]n2)nc1 ; Cl] |
| 7 | N-(3-(4-Methylpyridin-2-yl)-1H- pyrazol-5-yl)-4- morpholinopyrido[3′,2′:4,5]furo[3,2- d]pyrimidin-2-amine hydrochloride | 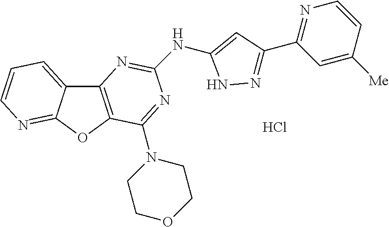 [SMILES: Cc1ccnc(-c2cc(Nc3nc(N4CCOCC4)c4oc5ncccc5c4n3)[nH]n2)c1 ; Cl] |
| 8 | N-(3-Methyl-1H-pyrazol-5-yl)-4- morpholinopyrido[3′,2′:4,5]furo[3,2- d]pyrimidin-2-amine | 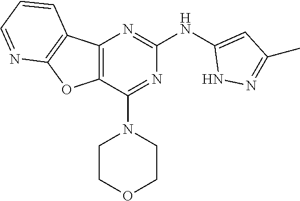 [SMILES: Cc1cc(Nc2nc(N3CCOCC3)c3oc4ncccc4c3n2)[nH]n1] |
| 9 | 4-Morpholino-N-(3-phenyl-1H- pyrazol-5- yl)pyrido[3′,2′:4,5]furo[3,2- d]pyrimidin-2-amine | 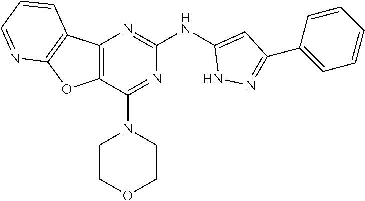 [SMILES: c1ccc(-c2cc(Nc3nc(N4CCOCC4)c4oc5ncccc5c4n3)[nH]n2)cc1] |
| 10 | 4-Morpholino-N-(3-(pyridin-4-yl)- 1H-pyrazol-5- yl)pyrido[3′,2′:4,5]furo[3,2- d]pyrimidin-2-amine | 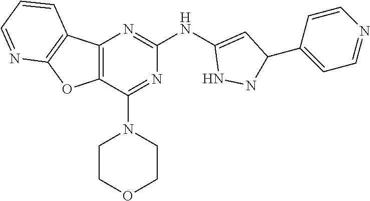 [SMILES: C1=C(Nc2nc(N3CCOCC3)c3oc4ncccc4c3n2)NNC1c1ccncc1] |
| 11 | N-(1,5-Dimethyl-1H-pyrazol-3-yl)- N-methyl-4- morpholinopyrido[3′,2′:4,5]furo[3,2- d]pyrimidin-2-amine | 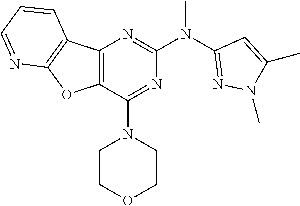 [SMILES: Cc1cc(N(C)c2nc(N3CCOCC3)c3oc4ncccc4c3n2)nn1C] |
| 12 | N-(1-Methyl-1H-imidazol-2-yl)-4- morpholinopyrido[3′,2′:4,5]furo[3,2- d]pyrimidin-2-amine | 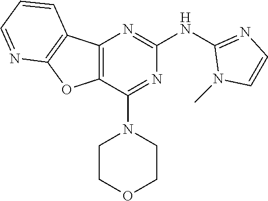 [SMILES: Cn1ccnc1Nc1nc(N2CCOCC2)c2oc3ncccc3c2n1] |
| 13 | 8-(2-(Dimethylamino)ethyl)-4- morpholino-N-(3-phenyl-1H- pyrazol-5- yl)pyrido[3′,2′:4,5]furo[3,2- d]pyrimidin-2-amine | 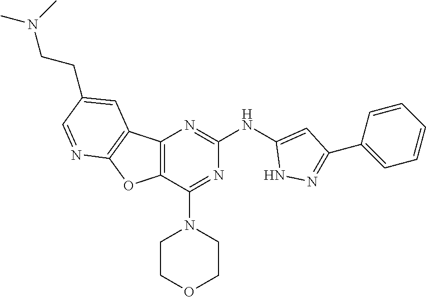 [SMILES: CN(C)CCc1cnc2oc3c(N4CCOCC4)nc(Nc4cc(-c5ccccc5)n[nH]4)nc3c2c1] |
| 14 | 4-Morpholino-N-(3-phenyl-1H- pyrazol-5-yl)-8-(2-(pyrrolidin-1- yl)ethyl)pyrido[3′,2′:4,5]furo[3,2- d]pyrimidin-2-amine | 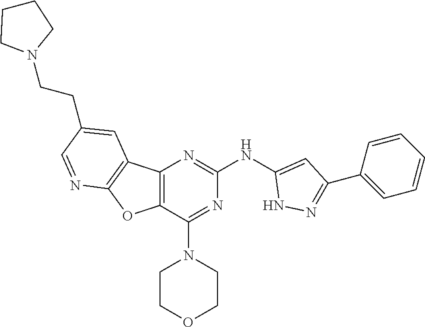 [SMILES: c1ccc(-c2cc(Nc3nc(N4CCOCC4)c4oc5ncc(CCN6CCCC6)cc5c4n3)[nH]n2)cc1] |
| 15 | N-(3-(2-fluorophenyl)-1H-pyrazol-5- yl)-4- morpholinopyrido[3′,2′:4,5]furo[3,2- d]pyrimidin-2-amine hydrochloride | 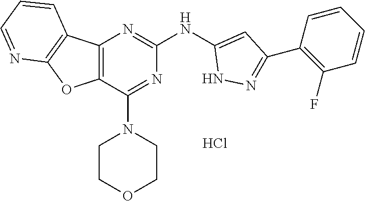 [SMILES: Fc1ccccc1-c1cc(Nc2nc(N3CCOCC3)c3oc4ncccc4c3n2)[nH]n1 ; Cl] |
| 16 | N-(3-(3-fluorophenyl)-1H-pyrazol-5- yl)-4- morpholinopyrido[3′,2′:4,5]furo[3,2- d]pyrimidin-2-amine hydrochloride | 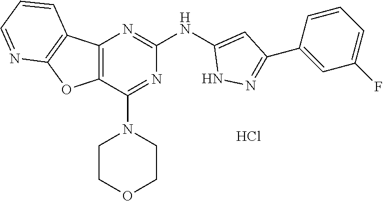 [SMILES: Fc1cccc(-c2cc(Nc3nc(N4CCOCC4)c4oc5ncccc5c4n3)[nH]n2)c1 ; Cl] |
| 17 | N-(3-(3-chlorophenyl)-1H-pyrazol-5- yl)-4- morpholinopyrido[3′,2′:4,5]furo[3,2- d]pyrimidin-2-amine hydrochloride | 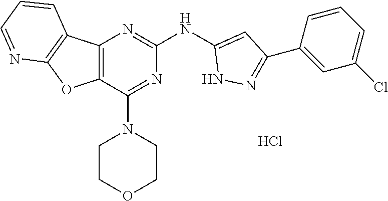 [SMILES: Clc1cccc(-c2cc(Nc3nc(N4CCOCC4)c4oc5ncccc5c4n3)[nH]n2)c1 ; Cl] |
| 18 | 4-morpholino-N-(3-(3- (trifluoromethyl)phenyl)-1H-pyrazol- 5-yl)pyrido[3′,2′:4,5]furo[3,2- d]pyrimidin-2-amine hydrochloride | 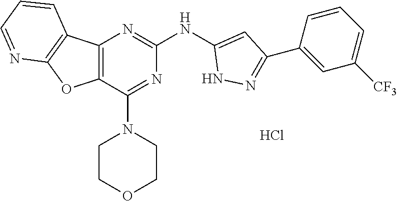 [SMILES: FC(F)(F)c1cccc(-c2cc(Nc3nc(N4CCOCC4)c4oc5ncccc5c4n3)[nH]n2)c1 ; Cl] |
| 19 | N-(3-(3,4-difluorophenyl)-1H-pyrazol- 5-yl)-4- morpholinopyrido[3′,2′:4,5]furo[3,2- d]pyrimidin-2-amine hydrochloride | 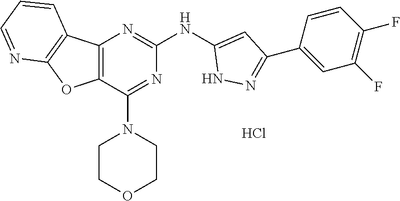 [SMILES: Fc1ccc(-c2cc(Nc3nc(N4CCOCC4)c4oc5ncccc5c4n3)[nH]n2)cc1F ; Cl] |
| 20 | 4-morpholino-N-(4-phenyl-1H- imidazol-2- yl)pyrido[3′,2′:4,5]furo[3,2- d]pyrimidin-2-amine hydrochloride | 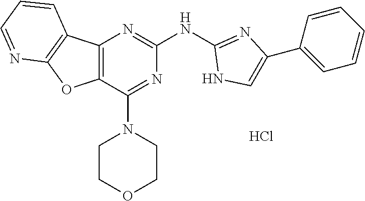 [SMILES: c1ccc(-c2c[nH]c(Nc3nc(N4CCOCC4)c4oc5ncccc5c4n3)n2)cc1 ; Cl] |
| 21 | 4-morpholino-N- phenethylpyrido[3′,2′:4,5]furo[3,2- d]pyrimidin-2-amine | 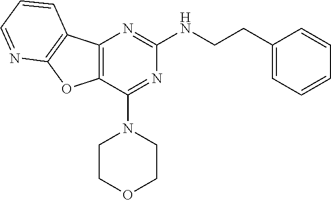 [SMILES: c1ccc(CCNc2nc(N3CCOCC3)c3oc4ncccc4c3n2)cc1] |
| 22 | phenyl(4- morpholinopyrido[3′,2′:4,5]furo[3,2- d]pyrimidin-2-yl)carbamate hydrochloride | 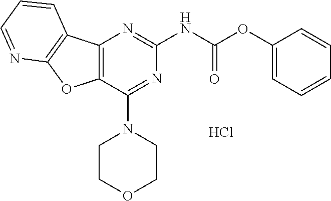 [SMILES: O=C(Nc1nc(N2CCOCC2)c2oc3ncccc3c2n1)Oc1ccccc1 ; Cl] |
| 23 | 7-(3-(dimethylamino)propoxy)-4- morpholino-N-(3-phenyl-1H-pyrazol- 5-yl)pyrido[3′,2′:4,5]furo[3,2- d]pyrimidin-2-amine hydrochloride | 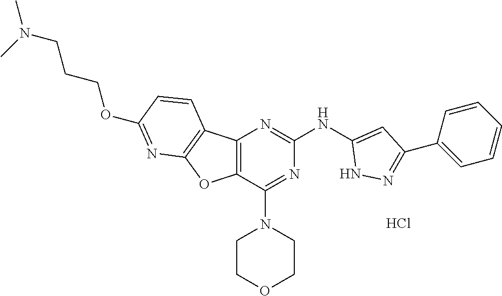 [SMILES: CN(C)CCCOc1ccc2c(n1)oc1c(N3CCOCC3)nc(Nc3cc(-c4ccccc4)n[nH]3)nc12 ; Cl] |
| 24 | 4-morpholino-7-(2- morpholinoethoxy)-N-(3-phenyl-1H- pyrazol-5-yl)pyrido[3′,2′:4,5]furo[3,2- d]pyrimidin-2-amine hydrochloride | 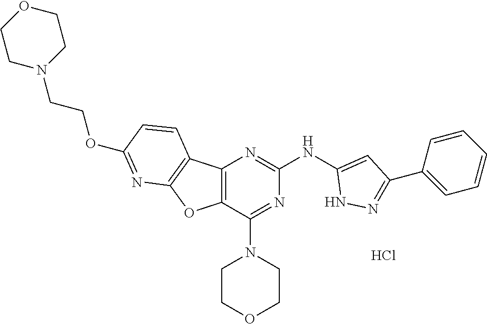 [SMILES: c1ccc(-c2cc(Nc3nc(N4CCOCC4)c4oc5nc(OCCN6CCOCC6)ccc5c4n3)[nH]n2)cc1 ; Cl] |
| 25 | 4-morpholino-7-(3- morpholinopropoxy)-N-(3-phenyl-1H- pyrazol-5-yl)pyrido[3′,2′:4,5]furo[3,2- d]pyrimidin-2-amine hydrochloride | 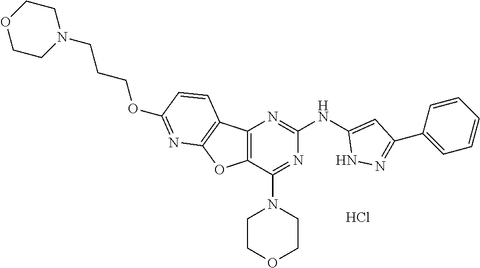 [SMILES: c1ccc(-c2cc(Nc3nc(N4CCOCC4)c4oc5nc(OCCCN6CCOCC6)ccc5c4n3)[nH]n2)cc1 ; Cl] |
| 26 | 4-morpholino-N-(3-phenyl-1H- pyrazol-5-yl)-7-(2-(pyrrolidin-1- yl)ethoxy)pyrido[3′,2′:4,5]furo[3,2- d]pyrimidin-2-amine hydrochloride | 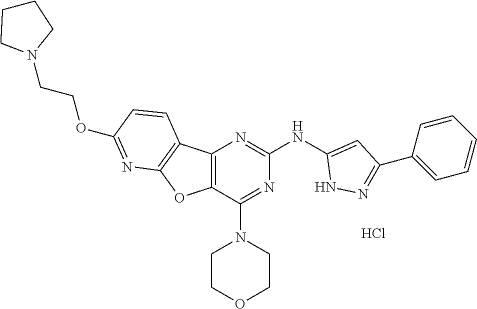 [SMILES: c1ccc(-c2cc(Nc3nc(N4CCOCC4)c4oc5nc(OCCN6CCCC6)ccc5c4n3)[nH]n2)cc1 ; Cl] |
| 27 | 4-morpholino-N-(3-phenyl-1H- pyrazol-5-yl)-7-(3-(pyrrolidin-1- yl)propoxy)pyrido[3′,2′:4,5]furo[3,2- d]pyrimidin-2-amine hydrochloride | 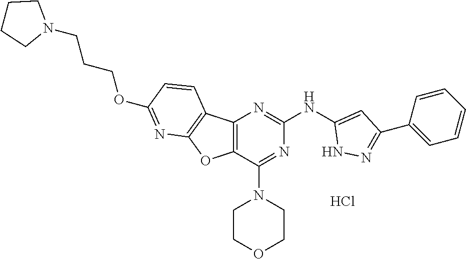 [SMILES: c1ccc(-c2cc(Nc3nc(N4CCOCC4)c4oc5nc(OCCCN6CCCC6)ccc5c4n3)[nH]n2)cc1 ; Cl] |
| 28 | 7-(2-(4-methylpiperazin-1-yl)ethoxy)- 4-morpholino-N-(3-phenyl-1H- pyrazol-5-yl)pyrido[3′,2′:4,5]furo[3,2- d]pyrimidin-2-amine hydrochloride | 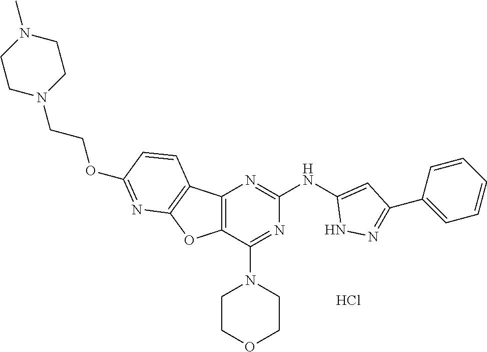 [SMILES: CN1CCN(CCOc2ccc3c(n2)oc2c(N4CCOCC4)nc(Nc4cc(-c5ccccc5)n[nH]4)nc23)CC1 ; Cl] |
| 29 | 7-(3-(4-methylpiperazin-1- yl)propoxy)-4-morpholino-N-(3- phenyl-1H-pyrazol-5- yl)pyrido[3′,2′:4,5]furo[3,2- d]pyrimidin-2-amine hydrochloride | 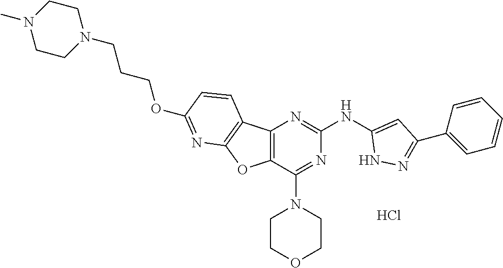 [SMILES: CN1CCN(CCCOc2ccc3c(n2)oc2c(N4CCOCC4)nc(Nc4cc(-c5ccccc5)n[nH]4)nc23)CC1 ; Cl] |
| 30 | 7-methoxy-4-morpholino-N-(3- phenyl-1H-pyrazol-5- yl)pyrido[3′,2′:4,5]furo[3,2- d]pyrimidin-2-amine hydrochloride | 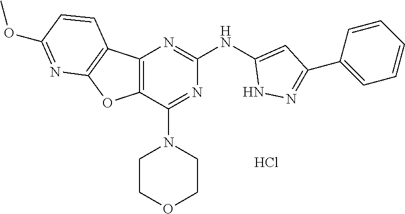 [SMILES: COc1ccc2c(n1)oc1c(N3CCOCC3)nc(Nc3cc(-c4ccccc4)n[nH]3)nc12 ; Cl] |
| 31 | 4-morpholino-N-(3-(pyridin-3-yl)-1H- pyrazol-5-yl)pyrido[3′,2′:4,5]furo[3,2- d]pyrimidin-2-amine hydrochloride | 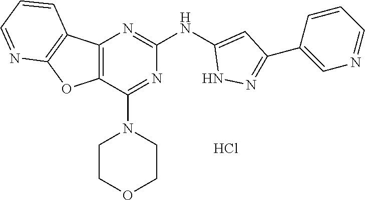 [SMILES: c1cncc(-c2cc(Nc3nc(N4CCOCC4)c4oc5ncccc5c4n3)[nH]n2)c1 ; Cl] |
| 32 | N-(3-(5-methylpyridin-3-yl)-1H- pyrazol-5-yl)-4- morpholinopyrido[3′,2′:4,5]furo[3,2- d]pyrimidin-2-amine hydrochloride | 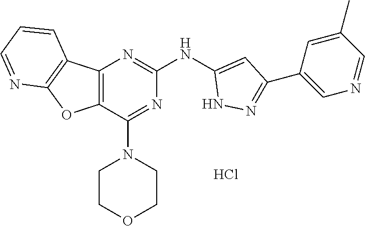 [SMILES: Cc1cncc(-c2cc(Nc3nc(N4CCOCC4)c4oc5ncccc5c4n3)[nH]n2)c1 ; Cl] |
| 33 | N-(3-(6-methylpyridin-2-yl)-1H- pyrazol-5-yl)-4- morpholinopyrido[3′,2′:4,5]furo[3,2- d]pyrimidin-2-amine hydrochloride | 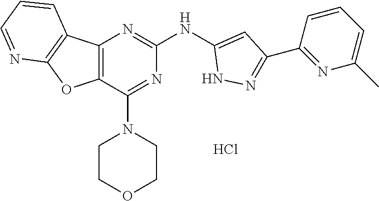 [SMILES: Cc1cccc(-c2cc(Nc3nc(N4CCOCC4)c4oc5ncccc5c4n3)[nH]n2)n1 ; Cl] |
| 34 | N-(3-(3-methoxyphenyl)-1H-pyrazol- 5-yl)-4- morpholinopyrido[3′,2′:4,5]furo[3,2- d]pyrimidin-2-amine hydrochloride | 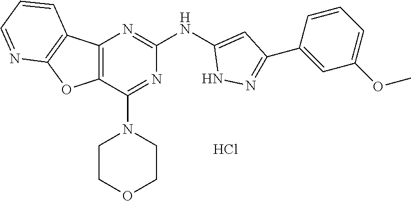 [SMILES: COc1cccc(-c2cc(Nc3nc(N4CCOCC4)c4oc5ncccc5c4n3)[nH]n2)c1 ; Cl] |
| 35 | 4-morpholino-N-(3-(3- (trifluoromethoxy)phenyl)-1H- pyrazol-5-yl)pyrido[3′,2′:4,5]furo[3,2- d]pyrimidin-2-amine hydrochloride | 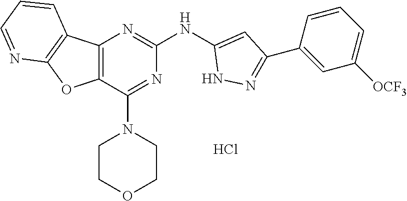 [SMILES: FC(F)(F)Oc1cccc(-c2cc(Nc3nc(N4CCOCC4)c4oc5ncccc5c4n3)[nH]n2)c1 ; Cl] |
| 36 | (S)-N-(1-cyclopropylethyl)-4- morpholinopyrido[3′,2′:4,5]furo[3,2- d]pyrimidin-2-amine | 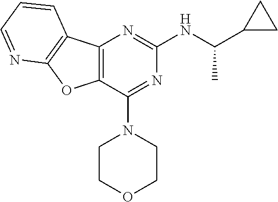 [SMILES: C[C@H](Nc1nc(N2CCOCC2)c2oc3ncccc3c2n1)C1CC1] |
| 37 | 4-morpholino-N- phenylpyrido[3′,2′:4,5]furo[3,2- d]pyrimidin-2-amine | 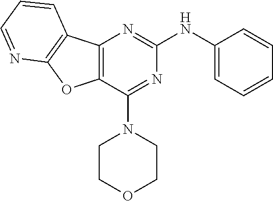 [SMILES: c1ccc(Nc2nc(N3CCOCC3)c3oc4ncccc4c3n2)cc1] |
| 38 | 4-morpholino-N-(pyridin-4- yl)pyrido[3′,2′:4,5]furo[3,2- d]pyrimidin-2-amine | 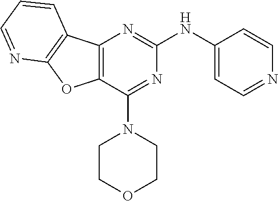 [SMILES: c1cnc2oc3c(N4CCOCC4)nc(Nc4ccncc4)nc3c2c1] |
| 39 | 4-morpholino-N-(pyridin-3- yl)pyrido[3′,2′:4,5]furo[3,2- d]pyrimidin-2-amine | 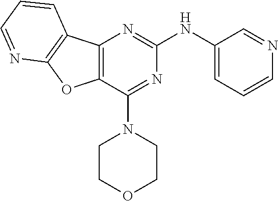 [SMILES: c1cncc(Nc2nc(N3CCOCC3)c3oc4ncccc4c3n2)c1] |
| 40 | 4-morpholino-N-(pyridin-2- yl)pyrido[3′,2′:4,5]furo[3,2- d]pyrimidin-2-amine | 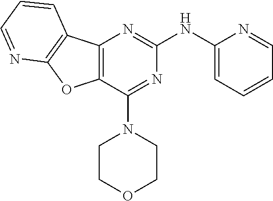 [SMILES: c1ccc(Nc2nc(N3CCOCC3)c3oc4ncccc4c3n2)nc1] |
| 41 | 4-morpholino-N-(pyrimidin-4- yl)pyrido[3′,2′:4,5]furo[3,2- d]pyrimidin-2-amine | 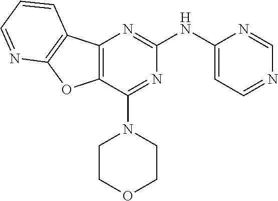 [SMILES: c1cnc2oc3c(N4CCOCC4)nc(Nc4ccncn4)nc3c2c1] |
| 42 | 4-morpholino-N-(pyrimidin-2- yl)pyrido[3′,2′:4,5]furo[3,2- d]pyrimidin-2-amine | 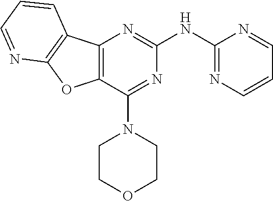 [SMILES: c1cnc(Nc2nc(N3CCOCC3)c3oc4ncccc4c3n2)nc1] |
| 43 | 4-morpholino-N-(pyrimidin-5- yl)pyrido[3′,2′:4,5]furo[3,2- d]pyrimidin-2-amine | 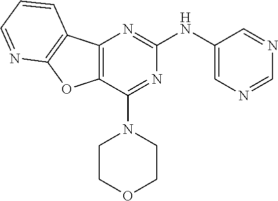 [SMILES: c1cnc2oc3c(N4CCOCC4)nc(Nc4cncnc4)nc3c2c1] |
| 44 | N-([1,1′-biphenyl]-4-yl)-4- morpholinopyrido[3′,2′:4,5]furo[3,2- d]pyrimidin-2-amine | 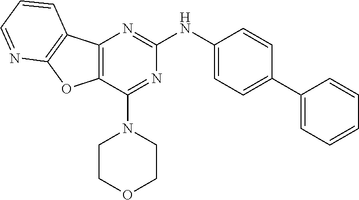 [SMILES: c1ccc(-c2ccc(Nc3nc(N4CCOCC4)c4oc5ncccc5c4n3)cc2)cc1] |
| 45 | N-([1,1′-biphenyl]-3-yl)-4- morpholinopyrido[3′,2′:4,5]furo[3,2- d]pyrimidin-2-amine | 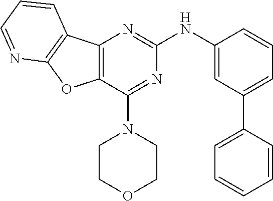 [SMILES: c1ccc(-c2cccc(Nc3nc(N4CCOCC4)c4oc5ncccc5c4n3)c2)cc1] |
| 46 | N-([1,1′-biphenyl]-2-yl)-4- morpholinopyrido[3′,2′:4,5]furo[3,2- d]pyrimidin-2-amine | 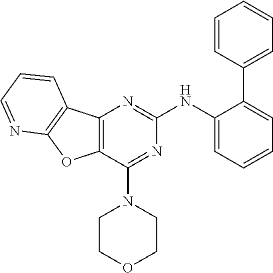 [SMILES: c1ccc(-c2ccccc2Nc2nc(N3CCOCC3)c3oc4ncccc4c3n2)cc1] |
| 47 | N-(1,5-dimethyl-1H-pyrazol-3-yl)-4- morpholinopyrido[3′,2′:4,5]furo[3,2- d]pyrimidin-2-amine | 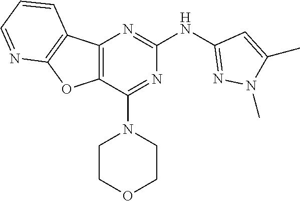 [SMILES: Cc1cc(Nc2nc(N3CCOCC3)c3oc4ncccc4c3n2)nn1C] |
| 48 | 4-morpholino-N-(3-(o-tolyl)-1H- pyrazol-5-yl)pyrido[3′,2′:4,5]furo[3,2- d]pyrimidin-2-amine | 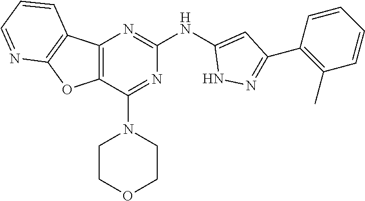 [SMILES: Cc1ccccc1-c1cc(Nc2nc(N3CCOCC3)c3oc4ncccc4c3n2)[nH]n1] |
| 49 | 4-morpholino-N-(3-(m-tolyl)-1H- pyrazol-5-yl)pyrido[3′,2′:4,5]furo[3,2- d]pyrimidin-2-amine | 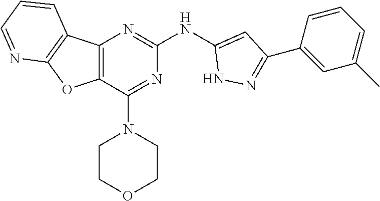 [SMILES: Cc1cccc(-c2cc(Nc3nc(N4CCOCC4)c4oc5ncccc5c4n3)[nH]n2)c1] |
| 50 | 4-morpholino-N-(3-(p-tolyl)-1H- pyrazol-5-yl)pyrido[3′,2′:4,5]furo[3,2- d]pyrimidin-2-amine | 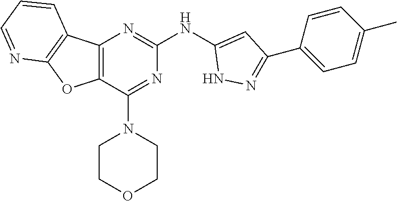 [SMILES: Cc1ccc(-c2cc(Nc3nc(N4CCOCC4)c4oc5ncccc5c4n3)[nH]n2)cc1] |
| 51 | 8-bromo-4-morpholino-N-(5-phenyl- 1H-pyrazol-3- yl)pyrido[3′,2′:4,5]furo[3,2- d]pyrimidin-2-amine | 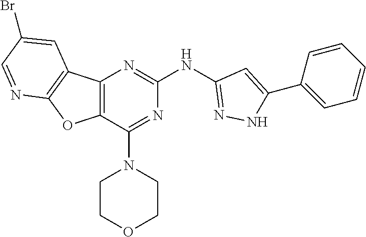 [SMILES: Brc1cnc2oc3c(N4CCOCC4)nc(Nc4cc(-c5ccccc5)[nH]n4)nc3c2c1] |
| 52 | 4- Morpholinopyrido[3′,2′:4,5]furo[3, 2-d]pyrimidine | 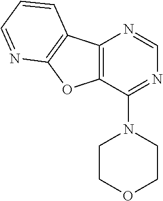 [SMILES: c1cnc2oc3c(N4CCOCC4)ncnc3c2c1] |
| 53 | N-(3-cyclopropyl-1H-pyrazol-5- yl)-4- morpholinopyrido[3′,2′:4,5]furo[3,2- d]pyrimidin-2-amine hydrochloride | 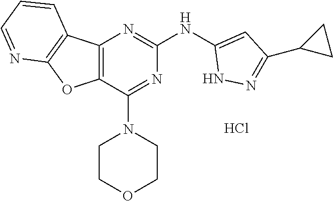 [SMILES: c1cnc2oc3c(N4CCOCC4)nc(Nc4cc(C5CC5)n[nH]4)nc3c2c1 ; Cl] |
| 54 | N-(3-(cyclopropylmethy1)-1H- pyrazol-5-yl)-4- morpholinopyrido[3′,2′:4,5]furo[3,2- d]pyrimidin-2-amine hydrochloride | 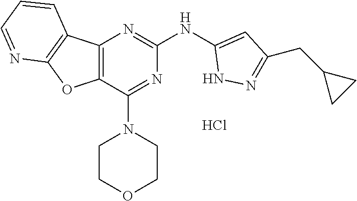 [SMILES: c1cnc2oc3c(N4CCOCC4)nc(Nc4cc(CC5CC5)n[nH]4)nc3c2c1 ; Cl] |
| 55 | N-(3-cyclobutyl-1H-pyrazol-5-yl)- 4- morpholinopyrido[3′,2′:4,5]furo[3,2- d]pyrimidin-2-amine hydrochloride | 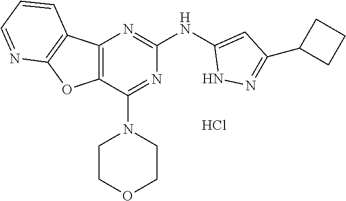 [SMILES: c1cnc2oc3c(N4CCOCC4)nc(Nc4cc(C5CCC5)n[nH]4)nc3c2c1 ; Cl] |
| 56 | N-(3-(cyclobutylmethyl)-1H- pyrazol-5-yl)-4- morpholinopyrido[3′,2′:4,5]furo[3,2- d]pyrimidin-2-amine hydrochloride | 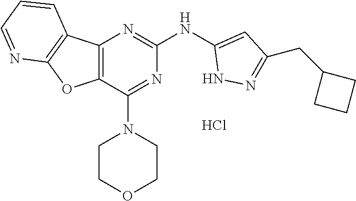 [SMILES: c1cnc2oc3c(N4CCOCC4)nc(Nc4cc(CC5CCC5)n[nH]4)nc3c2c1 ; Cl] |
| 57 | 4-morpholino-N-(3- (trifluoromethyl)-1H-pyrazol-5- yl)pyrido[3′,2′:4,5]furo[3,2- d]pyrimidin-2-amine hydrochloride | 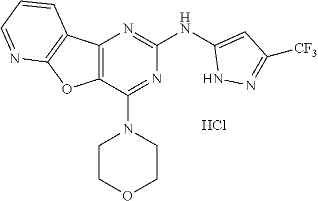 [SMILES: FC(F)(F)c1cc(Nc2nc(N3CCOCC3)c3oc4ncccc4c3n2)[nH]n1 ; Cl] |
| 58 | N-(3-benzyl-1H-pyrazol-5-yl)-4- morpholinopyrido[3′,2′:4,5]furo[3,2- d]pyrimidin-2-amine hydrochloride | 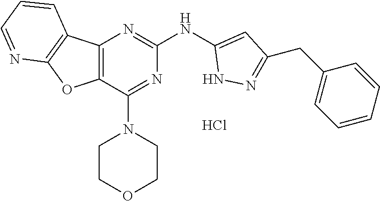 [SMILES: c1ccc(Cc2cc(Nc3nc(N4CCOCC4)c4oc5ncccc5c4n3)[nH]n2)cc1 ; Cl] |
| 59 | N-(3-cyclopentyl-1H-pyrazol-5-yl)- 4- morpholinopyrido[3′,2′:4,5]furo[3,2- d]pyrimidin-2-amine hydrochloride | 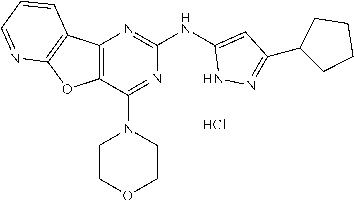 [SMILES: c1cnc2oc3c(N4CCOCC4)nc(Nc4cc(C5CCCC5)n[nH]4)nc3c2c1 ; Cl] |
| 60 | 4-morpholino-N-(3-(thiophen-2- yl)-1H-pyrazol-5- yl)pyrido[3′,2′:4,5]furo[3,2- d]pyrimidin-2-amine hydrochloride | 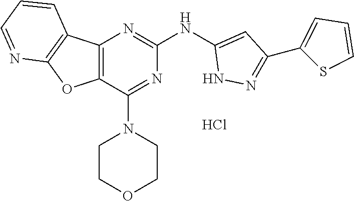 [SMILES: c1csc(-c2cc(Nc3nc(N4CCOCC4)c4oc5ncccc5c4n3)[nH]n2)c1 ; Cl] |
| 61 | 4-morpholino-N-(3-(thiophen-3- yl)-1H-pyrazol-5- yl)pyrido[3′,2′:4,5]furo[3,2- d]pyrimidin-2-amine hydrochloride | 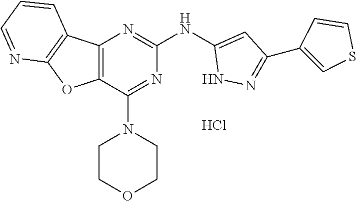 [SMILES: c1cnc2oc3c(N4CCOCC4)nc(Nc4cc(-c5ccsc5)n[nH]4)nc3c2c1 ; Cl] |
| 62 | N-(3-ethyl-1H-pyrazol-5-yl)-4- morpholinopyrido[3′,2′:4,5]furo[3,2- d]pyrimidin-2-amine hydrochloride | 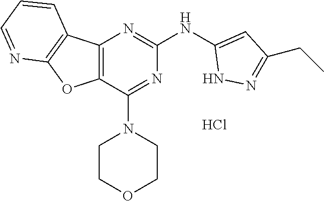 [SMILES: CCc1cc(Nc2nc(N3CCOCC3)c3oc4ncccc4c3n2)[nH]n1 ; Cl] |
| 63 | N-(3-isopropyl-1H-pyrazol-5-yl)-4- morpholinopyrido[3′,2′:4,5]furo[3,2- d]pyrimidin-2-amine hydrochloride | 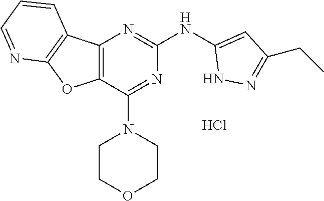 [SMILES: CCc1cc(Nc2nc(N3CCOCC3)c3oc4ncccc4c3n2)[nH]n1 ; Cl] |
| 64 | N-(3-(cyclopentylmethyl)-1H- pyrazol-5-yl)-4- morpholinopyrido[3′,2′:4,5]furo[3,2- d]pyrimidin-2-amin | 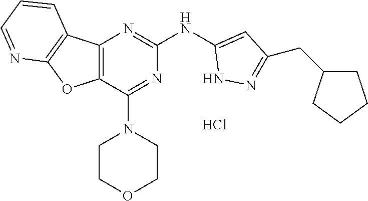 [SMILES: c1cnc2oc3c(N4CCOCC4)nc(Nc4cc(CC5CCCC5)n[nH]4)nc3c2c1 ; Cl] |

and pharmaceutically acceptable salts thereof.

### Methods of Treating, Administration, and Pharmaceutical Compositions

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. In one embodiment, the dosage level will be about 0.1 to about 250 mg/kg per day. In another embodiment the dosage level will be about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

Pharmaceutical compositions can be formulated using one or more pharmaceutically acceptable carriers comprising excipients and auxiliaries. The formulation can be modified depending upon the route of administration chosen. The pharmaceutical compositions can also include the compounds described herein in a free base form or a pharmaceutically acceptable salt form.

Methods for formulation of the pharmaceutical compositions can include formulating any of the compounds described herein with one or more inert, pharmaceutically acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(-)-3-hydroxybutyric acid.

Pharmaceutical formulations of the compositions described herein can be prepared for storage by mixing a compound with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together are safer or more effective than either drug alone. In some embodiments the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form. Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. In some embodiments, the combination agent is a drug for reduction of symptoms of ALS. In some embodiments, the combination agent is selected from an NAD supplement (such as nicotinamide riboside, offered under the trade names Basis® or Tru Niagen®), vitamin B₁₂ (oral or injection), glycopyrrolate, atropine, scopolamine, baclofen, tizanidine, mexiletine, an SSRI, a benzodiazepine, Neudexta, riluzole, and edaravone, and combinations thereof

The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human.

The compounds, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically effective amounts of the compounds or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition and can contemplate a range of results directed to that end, including but not restricted to prevention of the condition entirely.

Prevent, preventing, and the like can refer to the prevention of the disease or condition in the patient. For example, if an individual at risk of contracting a disease is treated with the methods of the present disclosure and does not later contract the disease, then the disease has been prevented, at least over a period of time, in that individual.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, compounds, and pharmaceutical compositions described herein can be for administration to a subject in need thereof. Often, administration of the compounds or pharmaceutical compositions can include routes of administration, non-limiting examples of administration routes include intravenous, intraarterial, subcutaneous, subdural, intramuscular, intracranial, intrasternal, or intraperitoneally. Additionally, a pharmaceutical composition or compound can be administered to a subject by additional routes of administration, for example, by inhalation, oral, dermal, intranasal, or intrathecal administration.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

The compounds, pharmaceutical compositions, and methods provided herein can be useful for the treatment of a plurality of diseases or conditions or preventing a disease or a condition in a subject, or other therapeutic applications for subjects in need thereof. In one aspect, the disclosure relates to a method for treating a neurological disease mediated by PIKfyve activity in a subject in need thereof, comprising administering an effective amount of a compound or a pharmaceutical composition as described herein to the subject. In some embodiments, the disease is associated with a FIG4 deficiency.

In some embodiments, the neurological disease is amyotrophic lateral sclerosis (ALS), primary lateral sclerosis (PLS), Charcot-Marie-Tooth (CMT; including type 4J (CMT4J)), and Yunis-Varon syndrome, autophagy, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, fronto-temporal dementia, diseases of neuronal nuclear inclusions of polyglutamine and intranuclear inclusion bodies, disease of Marinesco and Hirano bodies, tauopathy, Alzheimer's disease, neurodegeneration, spongiform neurodegeneration, peripheral neuropathy, leukoencephalopathy, motor neuropathy, sensory neuropathy, inclusion body disease, progressive supranuclear palsy, corticobasal syndrome, chronic traumatic encephalopathy, traumatic brain injury (TBI), cerebral ischemia, Guillain-Barré Syndrome, chronic inflammatory demyelinating polyneuropathy, multiple sclerosis, a lysosomal storage disease, Fabry's disorder, Gaucher's disorder, Niemann Pick C disease, Tay-Sachs disease, and Mucolipidosis type IV, neuropathy, Huntington's disease, a psychiatric disorder, ADHD, schizophrenia, a mood disorder, major depressive disorder, depression, bipolar disorder I, or bipolar disorder II.

In some embodiments, the neurological disease is ALS, FTD, Alzheimer's disease, Parkinson's disease, Huntington's disease, or CMT. In some embodiments, the neurological disease is ALS.

In some embodiments, the neurological disease is a tauopathy such as Alzheimer's disease, progressive supranuclear palsy, corticobasal syndrome, frontotemporal dementia, or chronic traumatic encephalopathy.

In some embodiments, the neurological disease is a lysosomal storage disease such as Fabry's disorder, Gaucher's disorder, Niemann Pick C disease, Tay-Sachs disease, or Mucolipidosis type IV.

In some embodiments, the neurological disease is a psychiatric disorder such as ADHD, schizophrenia, or mood disorders such as major depressive disorder, depression, bipolar disorder I, or bipolar disorder II.

In some aspects is a method of treating a disease mediated by PI3K activity in a subject in need thereof, comprising administering an effective amount of a compound or a pharmaceutical composition as described herein to the subject. In some embodiments, the PI3K is a PI3K isoform, such as PI3Kα, β, δ, and/or γ. In some embodiments, the disease is a neurological disease.

The disclosure further provides any compounds disclosed herein for use in a method of treatment of the human or animal body by therapy. Therapy may be by any mechanism disclosed herein, such as inhibiting, reducing, or reducing progression of the diseases disclosed herein. The disclosure further provides any compound disclosed herein for prevention or treatment of any condition disclosed herein. The disclosure also provides any compound or pharmaceutical composition thereof disclosed herein for obtaining any clinical outcome disclosed herein for any condition disclosed herein. The disclosure also provides use of any compound disclosed herein in the manufacture of a medicament for preventing or treating any disease or condition disclosed herein.

### EXAMPLES

The following preparations of compounds of Formula (I) and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about —78° C. to about 150° C., or from about 0° C. to about 125° C. or at about room (or ambient) temperature, e.g., about 20° C.

Compounds of Formula (I) and subformulae and species described herein, including those where the substituent groups as defined herein, can be prepared as illustrated and described below.

Unless otherwise noted, all reagents were used without further purification. ¹NMR spectra were obtained in CDCl₃, DMSO-d₆, or CD₃OD at room temperature on a Bruker 300 MHz instrument. When more than one conformer was detected, the chemical shifts for the most abundant one is reported. Chemical shifts of ¹H NMR spectra were recorded in parts per million (ppm) on the δ scale from an internal standard of residual solvent. Splitting patterns are designed as s, singlet; d, doublet; t, triplet; q, quartet; m, multiplet; br, broad. LC-MS conditions were described below:

### LCMS Column: Agilent Zorbax XDB C18 4.6×50 mm, 3.5μm

Mobile phase: Solvent A: Water (with 0.1% formic acid)
Solvent B: MeOH
   Flow rate: 1.0 mL/min,
   Run time: 2 min gradient (20%-90% B), then 3 min @90% B,

### Temperature: 30° C.

### HPLC Column: Agilent SB-C18 4.6×150 mm, 3.5 μm

Mobile phase: Solvent A: water (with 0.02% TFA)
Solvent B: MeOH
   Flow rate: 1.0 mL/min,
   Run time: 0.5 min @10% B, 9.5 min gradient (10%-90% B), then 10 min @90% B,

### Temperature: 30° C.

### Preparative LC Column: Phenomenex Luna 5u 100A, 21.2×250 mm, 5 μm

Mobile phase: Solvent A: Water
Solvent B: MeOH
   Flow rate: 10 mL/min,
   Run time: 1 min @20% B, 30 min gradient (20%-80% B), then 10 min @90% B,

### Temperature: Ambient

The following abbreviations are used in the text: PE=petroleum ether, EA or EtOAc=ethyl acetate, DMSO=dimethyl sulfoxide, DMF=N, N-dimethylacetamide, MeOH=methanol, DCM=dichloromethane, TFA=trifluoroacetic acid, TLC=thin layer chromatography, DME=1,2-dimethoxyethane.

### Example 1

### N-(3-(4-Fluorophenyl)-1H-pyrazol-5-yl)-4-morpholinopyrido[3′,2′:4,5]furo[3,2-d]pyrimidin-2-amine hydrochloride

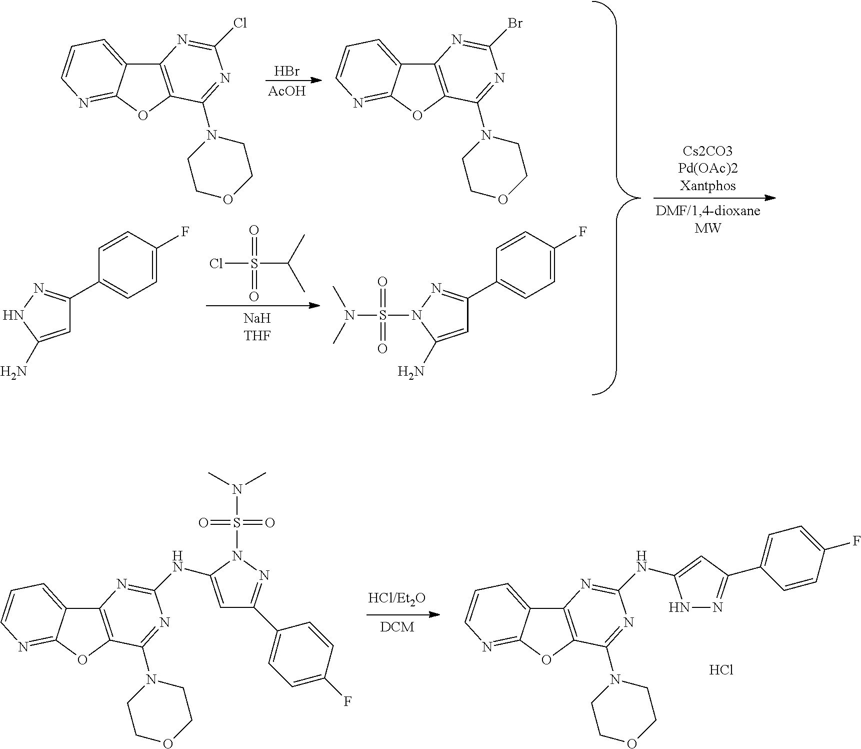

Step A: 2-Bromo-4-morpholinopyrido[3′,2′:4,5]furo[3,2-d]pyrimidine. A solution of 2-chloro-4-morpholinopyrido[3′,2′:4,5]furo[3,2-d]pyrimidine (2 g, 6.9 mmol) in HBr/AcOH (33 wt. % in acetic acid, 30 mL) was heated to reflux for 3.5 h. The reaction mixture was quenched with a saturated aq. NaHCO₃ solution and the pH was adjusted to 8. The aqueous solution was extracted with DCM/MeOH (15/1, 3×50 mL). The combined organic phase was dried over anhydrous Na₂SO₄, filtered, and concentrated to provide 2.19 g of crude product as yellow solid, which was used directly for the next step without further purification. LC-MS (ESI+): m/z 335/337 (MH⁺). ¹H NMR (300 MHz, DMSO-d₆) δ8.68 (dd, J=4.8, 1.5 Hz, 1H), 8.60 (dd, J=7.5, 1.5 Hz, 1H), 7.62 (dd, J=7.5, 1.8 Hz, 1H), 4.08-3.95 (m, 4H), 3.88-3.78 (m, 4H).

Step B: 5-Amino-3-(4-fluorophenyl)-N,N-dimethyl-1H-pyrazole-1-sulfonamide. To a solution of 3-(4-fluorophenyl)-1H-pyrazol-5-amine (300 mg, 1.69 mmol) in THF (5 mL) at 0° C. was added NaH (100 mg, 2.54 mmol). The solution was stirred at that temperature for 1 h. To the above suspension was added dimethylsulfamoyl chloride (315 mg, 2.20 mmol). A saturated aq. NH₄Cl solution was added and the aqueous solution was extracted with ethyl acetate (3×50 mL). The combined organic phase was dried over anhydrous Na₂SO₄, filtered, and concentrated. The residue was purified by silica gel column chromatography with a gradient elution of 20% EtOAc/PE to 33% EtOAc/PE to provide 5-amino-3-(4-fluorophenyl)-N,N-dimethyl-1H-pyrazole-1-sulfonamide (310 mg, 1.09 mmol). LC-MS: m/z 285 (MH⁺). ¹H NMR (300 MHz, CDCl₃) δ 7.74 (t, J=8.4 Hz, 2H), 7.07 (t, J=8.7 Hz, 2H), 5.70 (s, 1H), 4.85 (s, 2H), 3.03 (s, 6H).

Step C: 3-(4-Fluorophenyl)-N,N-dimethyl-5-((4-orpholinopyrido[3′,2′:4,5]furo[3,2-d]pyrimidin-2-yl)amino)-1H-pyrazole-1-sulfonamide. A solution of 2-bromo-4-morpholinopyrido[3′,2′:4,5]furo[3,2-d]pyrimidine (60 mg, 0.18 mmol), 5-amino-3-(4-fluorophenyl)-N,N-dimethyl-1H-pyrazole-1-sulfonamide (61.2 mg, 0.22 mmol), Cs₂CO₃ (134.4 mg, 0.41 mmol), Pd(OAc)₂(4.2 mg, 0.018 mmol) and Xantphos (10.2 mg, 0.018 mmol) in DMF/1,4-dioxane (7/1, 5 mL) was heated to 90° C. for 30 min under microwave conditions. The reaction mixture was concentrated directly and purified by silica gel column chromatography with a gradient elution of 2% MeOH/DCM to 3% MeOH/DCM to provide 3-(4-fluorophenyl)-N,N-dimethyl-5-((4-morpholinopyrido[3′,2′:4,5]furo[3,2-d]pyrimidin-2-yl)amino)-1H-pyrazole-1-sulfonamide (58.6 mg, 0.11 mmol) as a yellow solid. LC-MS: m/z 539 (MH⁺) ¹H NMR (300 MHz, CDCl₃) δ 8.81 (s, 1H), 8.59 (d, J=3.0 Hz, 1H), 8.49 (d, J=8.1 Hz, 1H), 7.89-7.86 (m, 2H), 7.46-7.40 (m, 1H), 7.30 (s, 1H), 7.15 (t, J=8.7 Hz, 2H), 4.18-4.12 (m, 4H), 3.91-3.85 (m, 4H), 3.09 (s, 6H).

Step D. To a solution of 3-(4-fluorophenyl)-N,N-dimethyl-5-((4-morpholinopyrido[3′,2′:4,5]furo[3,2-d]pyrimidin-2-yl)amino)-1H-pyrazole-l-sulfonamide (58.6 mg, 0.11 mmol) in DCM (2 mL) was added HCl/Et₂O (1 mL). The reaction was stirred for 2 h and a large amount of solid was precipitated. After concentration and slurry in MeOH/Et₂O (1/20, 2 mL), N-(3-(4-fluorophenyl)-1H-pyrazol-5-yl)-4-morpholinopyrido[3′,2′:4,5]furo[3,2-d]pyrimidin-2-amine hydrochloride (35 mg, 0.08 mmol) was obtained as a white solid. LC-MS: m/z 432 (MH⁺) ¹H NMR (300 MHz, DMSO-d₆) δ 9.94 (s, 1H), 8.78 (d, J=8.7 Hz, 1H), 8.64 (d, J=8.7 Hz, 1H), 7.86 (t, J=5.7 Hz, 2H), 7.60-7.56 (m, 1H), 7.31 (t, J=8.4 Hz, 2H), 6.66 (s, 1H), 4.12-4.05 (m, 4H), 3.88-3.82 (m, 4H).

### Example 2

### N-Benzyl-4-morpholinopyrido[3′,2′:4,5]furo[3,2-d]pyrimidin-2-amine.

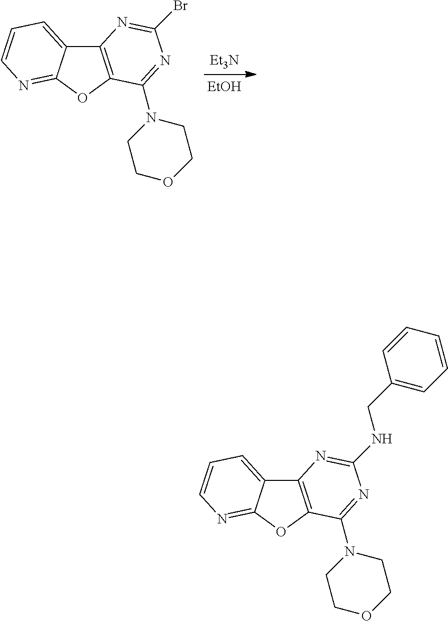 [SMILES: "Et3N" ; "EtOH" ; Brc1nc(N2CCOCC2)c2oc3ncccc3c2n1 ; c1ccc(CNc2nc(N3CCOCC3)c3oc4ncccc4c3n2)cc1]

To a solution of 2-bromo-4-morpholinopyrido[3′,2′:4,5]furo[3,2-d]pyrimidine (60 mg, 0.18 mmol) in EtOH (30 mL) was added Et₃N (36 mg, 0.36 mmol) and phenylmethanamine (20 mg, 0.18 mmol). The reaction was heated to 120° C. for 50 min under microwave conditions. The reaction mixture was concentrated directly and the resulting residue was purified by silica gel column chromatography with a gradient elution of 2% MeOH/DCM to 3% MeOH/DCM to provide N-benzyl-4-morpholinobenzofuro[3,2-d]pyrimidin-2-amine (25 mg, 0.07 mmol) as an off-white solid. LC-MS (ESI+): m/z 362 (MH⁺). ¹H NMR (300 MHz, CDCl₃) δ 8.52 (d, J=3.3 Hz, 1H), 8.50 (dd, J=7.8, 1.8 Hz, 1H), 7.39-7.27 (m, 6H), 5.42-5.31 (m, 1H), 4.67 (d, J=5.7 Hz, 2H), 4.05-4.01 (m, 4H), 3.82-3.79 (m, 4H).

### Example 3

### N-(4-Morpholinobenzofuro[3,2-d]pyrimidin-2-yl)benzamide.

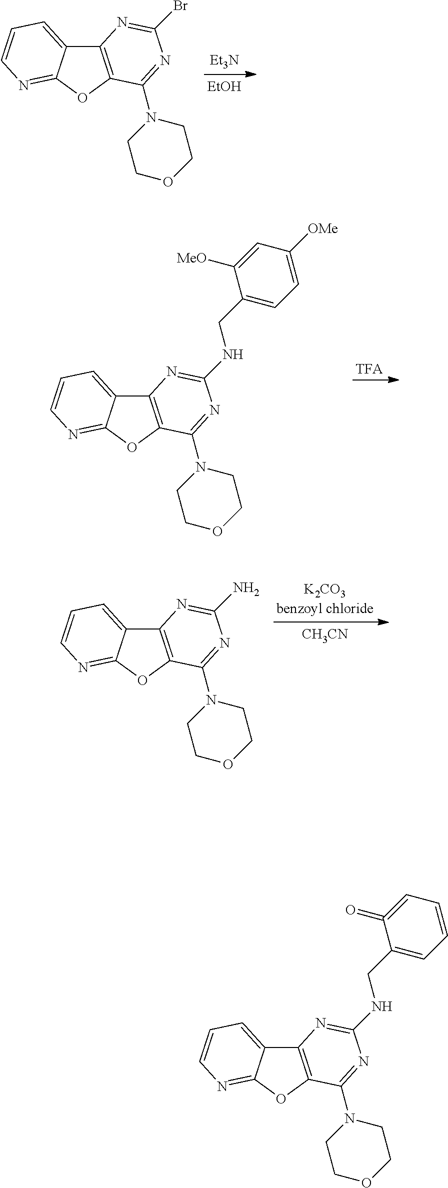

Step A: N-(2,4-Dimethoxybenzyl)-4-morpholinobenzofuro[3,2-d]pyrimidin-2-amine. To a solution of 2-bromo-4-morpholinobenzofuro[3,2-d]pyrimidine (300 mg, 0.89 mmol) in EtOH (20 mL) was added (2,4-dimethoxyphenyl)methanamine (300 mg, 1.79 mmol) and Et₃N (272 mg, 2.69 mmol). The reaction was heated to 120° C. for 60 min under microwave conditions. The reaction mixture was concentrated directly and the resulting residue was purified by silica gel column chromatography with a gradient elution of 1% MeOH/DCM to 3% MeOH/DCM to provide N-(2,4-dimethoxybenzyl)-4-morpholinobenzofuro[3,2-d]pyrimidin-2-amine (188 mg, 0.44 mmol). LC-MS: m/z 422 (MH⁺) ¹H NMR (300 MHz, CDCl₃) δ 8.50 (d, J =4.8 Hz, 1H), 8.38 (dd, J=7.5, 1.8 Hz, 1H), 7.36-7.32 (m, 2H), 6.47 (d, J=2.4 Hz, 1H), 6.42 (dd, J=8.4, 2.4 Hz, 1H), 5.33-5.28 (m, 1H), 4.57 (d, J=6.3 Hz, 2H), 4.08-4.05 (m, 4H), 3.85-3.82 (m, 7H), 3.79 (s, 3H).

Step B: 4-Morpholinobenzofuro[3,2-d]pyrimidin-2-amine. A solution of N-(2,4-dimethoxybenzyl)-4-morpholinobenzofuro[3,2-d]pyrimidin-2-amine (183 mg, 0.43 mmol) in TFA (5 mL) was heated to 60° C. for 1 h. A saturated aq. NaHCO₃ solution was added to adjust the pH to 9. The aqueous solution was extracted with MeOH/DCM (1/15, 3×10 mL). The combined organic phase was dried over anhydrous Na₂SO₄, filtered, and concentrated to provide crude 4-morpholinobenzofuro[3,2-d]pyrimidin-2-amine (200 mg, 0.74 mmol) which was used directly for next step without further purification. LC-MS: m/z 272 (MH⁺). ¹H NMR (300 MHz, CDCl₃) δ 8.53 (dd, J=4.8, 1.8 Hz, 1H), 8.38 (dd, J=7.5, 1.8 Hz, 1H), 7.37 (dd, J=7.8, 4.8 Hz, 1H), 4.76 (s, 2H), 4.08-4.05 (m, 4H), 3.86-3.81 (m, 4H).

Step C. To a suspension of 4-morpholinobenzofuro[3,2-d]pyrimidin-2-amine (20 mg, 0.07 mmol) and K₂CO₃ (25 mg, 0.18 mmol) in CH₃CN at rt was added benzoyl chloride (20 mg, 0.14 mmol). The reaction mixture was stirred at rt overnight. The reaction mixture was quenched with H₂O (5 mL) and the aqueous was extracted with MeOH/DCM (1/15, 3×10 mL). The combined organic phase was dried over anhydrous Na₂SO₄, filtered, and concentrated. The resulting residue was purified by preparative TLC to provide N-(4-morpholinobenzofuro[3,2-d]pyrimidin-2-yl)benzamide (23 mg, 0.06 mmol) as a white solid. LC-MS: m/z 376 (MH⁺). ¹H NMR (300 MHz, CDCl₃) δ 8.60-8.58 (m, 1H), 8.54-8.49 (m, 2H), 7.94 (d, J=6.6 Hz, 2H), 7.61-7.46 (m, 3H), 4.20-4.10 (m, 4H), 3.89-3.86 (m, 4H).

### Example 4

### 7-(2-(Dimethylamino)ethoxy)-4-morpholino-N-(3-phenyl-1H-pyrazol-5-yl)pyrido[3′,2′:4,5]furo[3,2-d]pyrimidin-2-amine hydrochloride.

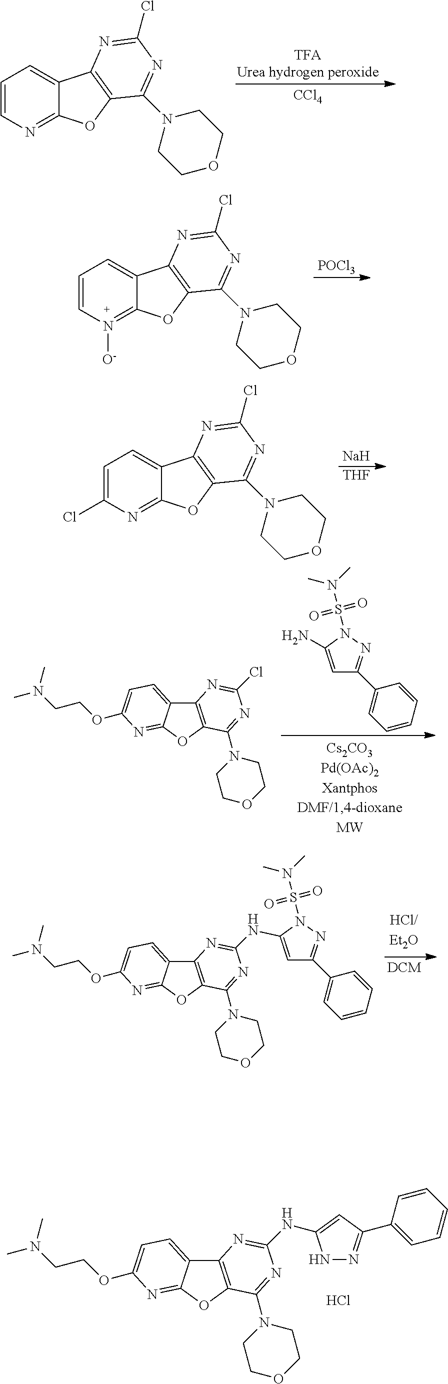

Step A: 2-Chloro-4-morpholinopyrido[3′,2′:4,5]furo[3,2-d]pyrimidine 6-oxide. To a solution of 2-chloro-4-morpholinopyrido[3′,2′:4,5]furo[3,2-d]pyrimidine (5 g, 3.45 mmol) and urea hydrogen peroxide (8.1 g, 17.24 mmol) in CCl₄ (150 mL) at 0° C. was added TFA (6.5 mL, 17.24 mmol) dropwise using addition funnel. After addition, the reaction was heated to reflux overnight. After the reaction mixture was cooled to rt, saturated aq. NaHCO₃ was added to adjust pH to 8. The resulting biphasic mixture was transferred to a separatory funnel. The layers were separated, and the aqueous phase was extracted with DCM (5×50 mL). The combined organic phase was dried over anhydrous Na₂SO₄, filtered, and concentrated under reduced pressure. The resulting residue was purified by silica gel column chromatography with a gradient elution of 2% MeOH/DCM to 4% MeOH/DCM to provide 2-chloro-4-morpholinopyrido[3′,2′:4,5]furo[3,2-d]pyrimidine 6-oxide (420 mg, 1.37 mmol) as light yellow solid and 2 g of 2-chloro-4-morpholinopyrido[3′,2′:4,5]furo[3,2-d]pyrimidine was recovered. LC-MS (ESI+): m/z 307/309 (M⁺).

Step B: 2,7-Dichloro-4-morpholinopyrido[3′,2′:4,5]furo[3,2-d]pyrimidine. A solution of 2-chloro-4-morpholinopyrido[3′,2′:4,5]furo[3,2-d]pyrimidine 6-oxide (420 mg, 1.37 mmol) in POCl₃ (15 mL) was heated to 110° C. for 3 h. The reaction mixture was concentrated directly to remove the excess amount of POCl₃. To the resulting residue was added saturated aq. NaHCO₃ to adjust pH to 8. The aqueous solution was extracted with MeOH/DCM (1/20, 3×20 mL). The combined organic phase was dried over anhydrous Na₂SO₄, filtered, and concentrated under reduced pressure. The resulting residue was purified by silica gel column chromatography with a gradient elution of 20% EtOAc/PE to 33% EtOAc/PE to provide 160 mg of a mixture of the target compound and the 2,9-dichloro isomer as a white solid. LC-MS (ESI+): m/z 325/327 (MH⁺).

Step C: 2-((2-Chloro-4-morpholinopyrido[3′,2′:4,5]furo[3,2-d]pyrimidin-7-yl)oxy)-N,N-dimethylethanamine. To a solution of the mixture from Step B (80 mg, 0.25 mmol) in THF (30 mL) at 0° C. was added NaH (20 mg, 0.5 mmol). After stirring at 0° C. for 20 min, a solution of 2-(dimethylamino)ethanol (33 mg, 0.37 mmol) in THF was added. After addition, the reaction mixture was warmed to rt and stirred for 2 h. The reaction mixture was quenched with saturated aq. NH₄Cl. The aqueous solution was extracted with EtOAc (3×20 mL). The combined organic phase was dried over anhydrous Na₂SO₄, filtered, and concentrated under reduced pressure. The resulting residue was purified by silica gel column chromatography with a gradient elution of 20% EtOAc/PE to 33% EtOAc/PE to provide the target compound (49 mg, 0.13 mmol, upper spot) as white solid. LC-MS (ESI+): m/z 378/380 (MH⁺) ^{1l}H NMR (300 MHz, CDCl₃) δ 8.30 (d, J=8.4 Hz, 1H), 6.92 (d, J=8.4 Hz, 1H), 4.55 (t, J=5.4 Hz, 2H), 4.16-4.08 (m, 4H), 3.91-3.84 (m, 4H), 2.83 (t, J=5.1 Hz, 2H), 2.45 (s, 6H).

Step D: 5-Amino-N,N-dimethyl-3-phenyl-1H-pyrazole-1-sulfonamide. To a solution of 3-phenyl-1H-pyrazol-5-amine (300 mg, 1.88 mmol) in THF (5 mL) at 0° C. was added NaH (100 mg, 2.82 mmol). After stirring at 0° C. for 1 h, to the solution was added dimethylsulfamoyl chloride (315 mg, 2.20 mmol). The reaction mixture was quenched with saturated aq. NH₄Cl. The aqueous solution was extracted with ethyl acetate (3×50 mL). The combined organic phase was dried over anhydrous Na₂SO₄, filtered, and concentrated under reduced pressure. The resulting residue was purified by silica gel column chromatography with a gradient elution of 20% EtOAc/PE to 33% EtOAc/PE to provide the title compound (300 mg, 1.13 mmol). LC-MS: m/z 267 (MH⁺). ¹H NMR (300 MHz, CDCl₃) δ 7.79-7.76 (m, 2H), 7.42-7.35 (m, 3H), 5.75 (s, 1H), 4.84 (s, 2H), 3.03 (s, 6H).

Step E: 5-((7-(2-(Dimethylamino)ethoxy)-4-morpholinopyrido[3′,2′:4,5]furo[3,2-d]pyrimidin-2-yl)amino)-N,N-dimethyl-3-phenyl-1H-pyrazole-1-sulfonamide. A solution of 2-((2-chloro-4-morpholinopyrido[3′,2′:4,5]furo[3,2-d]pyrimidin-7-yl)oxy)-N,N-dimethylethanamine (35 mg, 0.09 mmol), 5-amino-N,N-dimethyl-3-phenyl-1H-pyrazole-1-sulfonamide (37 mg, 0.14 mmol), Cs₂CO₃ (76 mg, 0.23 mmol), Pd(OAc)₂ (2 mg, 0.01 mmol) and Xantphos (5 mg, 0.01 mmol) in DMF/1,4-dioxane (7/1, 4 mL) was heated to 95° C. for 60 min under microwave conditions. The reaction was diluted with H₂O (10 mL) and extracted with ethyl acetate (3×15 mL). The combined organic phase was dried over anhydrous Na₂SO₄, filtered, and concentrated under reduced pressure. The resulting residue was purified by silica column chromatography with a gradient elution from 1% MeOH /DCM to 2% MeOH /DCM to provide the title compound (39 mg, 0.06 mmol) as a white solid. LC-MS (ESI+): m/z 608 (MH⁺). ¹H NMR (300 MHz, CDCl₃) δ 8.77 (s, 1H), 8.31 (d, J=8.1 Hz, 1H), 7.90 (d, J=6.6 Hz, 2H), 7.48-7.39 (m, 3H), 7.32 (s, 1H), 6.94 (d, J=8.4 Hz, 1H), 4.53 (t, J=5.7 Hz, 2H), 4.13-4.07 (m, 4H), 3.93-3.86 (m, 4H), 3.07 (s, 6H), 2.79 (t, J=5.4 Hz, 2H), 2.37 (s, 6H).

Step F. To a solution of 5-((7-(2-(dimethylamino)ethoxy)-4-morpholinopyrido[3′,2′:4,5]furo[3,2-d] pyrimidin-2-yl)amino)-N,N-dimethyl-3 -phenyl- 1H-pyrazole-1-sulfonamide (39 mg, 0.06 mmol) in DCM (2 mL) was added a solution of HCl/Et₂O (1 mL). The reaction was stirred at rt for 2 h. The reaction mixture was concentrated directly and the resulting residue was slurried in MeOH/Et₂O (1/20, 5 mL) to provide the title compound (37 mg, 0.07 mmol) as a white solid. LC-MS (ESI+): m/z 501 (MH⁺). ¹H NMR (300 MHz, CD₃OD) δ 8.54 (d, J=8.4 Hz, 1H), 7.73 (d, J=8.4 Hz, 2H), 7.53-7.44 (m, 3H), 7.22 (d, J=8.7 Hz, 1H), 6.49 (s, 1H), 4.32-4.27 (m, 4H), 3.96-3.88 (m, 4H), 3.70-3.63 (m, 2H), 3.37-3.30 (m, 2H), 3.03 (s, 6H).

### Example 5

### 4-Morpholino-2-((3-phenyl-1H-pyrazol-5-yl)amino)pyrido[3′,2′:4,5]furo[3,2-d]pyrimidin-7-ol hydrochloride.

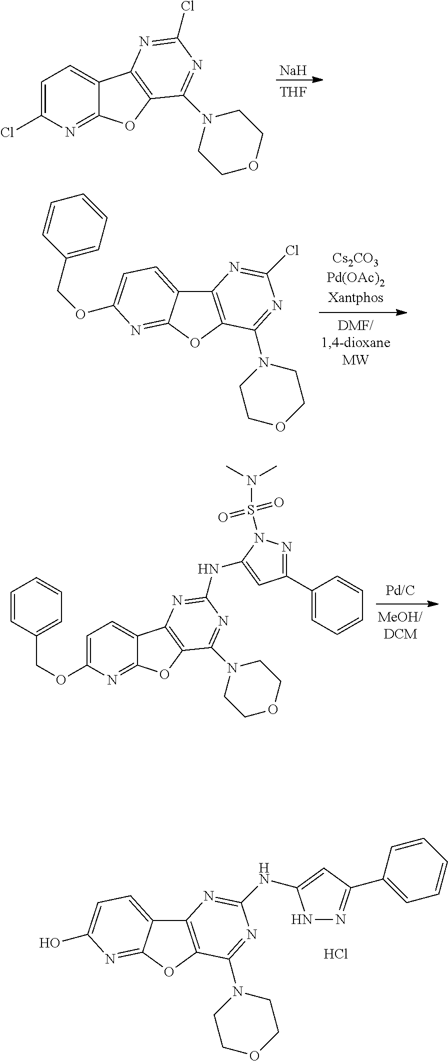

Step A: 7-(Benzyloxy)-2-chloro-4-morpholinopyrido[3′,2′:4,5]furo[3,2-d]pyrimidine. To a solution of 2,7-dichloro-4-morpholinopyrido[3′,2′:4,5]furo[3,2-d]pyrimidine (150 mg, 0.46 mmol) in THF (30 mL) at 0° C. was added NaH (92 mg, 2.3 mmol). The mixture was stirred at 0 ° C. for 20 min. To the reaction mixture was added a solution of phenylmethanol (60 mg, 0.56 mmol) in THF. After addition, the reaction mixture was warmed to rt and stirred for 2 h. The reaction mixture was quenched with saturated aq. NH₄Cl. The aqueous solution was extracted with ethyl acetate (3×20 mL). The combined organic phase was dried over anhydrous Na₂SO₄, filtered, and concentrated under reduced pressure. The resulting residue was purified by silica gel column chromatography with a gradient elution of 10% EtOAc/PE to 20% EtOAc/PE to provide the title compound (110 mg, 0.28 mmol) as a white solid. LC-MS (ESI+): m/z 397/399 (MH⁺). ¹H NMR (300 MHz, CDCl₃) δ 8.33 (d, J=8.4 Hz, 1H), 7.50-7.31 (m, 5H), 6.95 (d, J=8.4 Hz, 1H), 5.49 (s, 2H), 4.13-4.10 (m, 4H), 3.89-3.86 (m, 4H).

Step B: 5-((7-(benzyloxy)-4-morpholinopyrido[3′,2′:4,5]furo[3,2-d]pyrimidin-2-yl)amino)-N,N-dimethyl-3-phenyl-1H-pyrazole-1-sulfonamide. A solution of 7-(benzyloxy)-2-chloro-4-morpholinopyrido[3′,2′:4,5]furo[3,2-d]pyrimidine (100 mg, 0.25 mmol), 5-amino-N,N-dimethyl-3-phenyl-1H-pyrazole-1-sulfonamide (101 mg, 0.38 mmol), Cs₂CO₃ (165 mg, 0.51 mmol), Pd(OAc)₂ (6 mg, 0.03 mmol), and Xantphos (15 mg, 0.03 mmol) in DMF/1,4-dioxane (7/1, 2.4 mL) was heated to 95° C. for 60 min under microwave conditions. The reaction mixture was concentrated directly under reduced pressure. The resulting residue was purified by silica gel column chromatography with a gradient elution of 1% MeOH/DCM to 2% MeOH/DCM to provide 5-((7-(benzyloxy)-4-morpholinopyrido[3′,2′:4,5] furo[3,2-d]pyrimidin-2-yl)amino)-N,N-dimethyl-3-phenyl-1H-pyrazole-1-sulfonamide (62 mg, 0.09 mmol) as a light yellow solid. LC-MS (ESI+): m/z 627 (MH⁺). ¹HNMR (300 MHz, CDCl₃) δ 8.78 (s, 1H), 8.34 (d, J=8.4 Hz, 1H), 8.90 (d, J=6.9 Hz, 2H), 7.52-7.32 (m, 9H), 6.95 (d, J=8.4 Hz, 1H), 5.50 (s, 2H), 4.15-4.10 (m, 4H), 3.93-3.88 (m, 4H), 3.08 (s, 6H).

Step C: 5-((7-Hydroxy-4-morpholinopyrido[3′,2′:4,5]furo[3,2-d]pyrimidin-2-yl)amino)-N,N-dimethyl-3-phenyl-1H-pyrazole-1-sulfonamide. To a solution of 5-((7-(benzyloxy)-4-morpholinopyrido[3′,2′:4,5]furo[3,2-d]pyrimidin-2-yl)amino)-N,N-dimethyl-3-phenyl-1H-pyrazole-1-sulfonamide (62 mg, 0.09 mmol) in MeOH/DCM (1/4, 5 mL) was added Pd/C (10 mg) and then bubbled with Musing balloon for 2 h. After filtration, the reaction mixture was concentrated directly under reduced pressure. The resulting residue was purified by silica gel column chromatography with a gradient elution of 1% MeOH/DCM to 2% MeOH/DCM to provide the title compound (31 mg, 0.06 mmol) as a white solid. LC-MS (ESI+): m/z 537 (MH⁺). ¹H NMR (300 MHz, CDCl₃) δ 8.81 (s, 1H), 8.39 (d, J=8.7 Hz, 1H), 8.90 (d, J=6.6 Hz, 2H), 7.46-7.40 (m, 3H), 7.30 (s, 1H), 6.91 (d, J=8.4 Hz, 1H), 4.12-4.07 (m, 4H), 3.92-3.87 (m, 4H), 3.08 (s, 6H).

Step D. To a solution of 5-((7-hydroxy-4-morpholinopyrido[3′,2′:4,5]furo[3,2-d]pyrimidin-2-yl)amino)-N,N-dimethyl-3-phenyl-1H-pyrazole-1-sulfonamide (31 mg, 0.06 mmol) in DCM (2 mL) was added a solution of HCl/Et₂O (1 mL). The reaction mixture was stirred at rt for 2 h. The reaction mixture was concentrated directly and the resulting residue was slurried in MeOH/Et₂O(1/20, 5 mL) to provide the title compound (18.2 mg, 0.04 mmol) as a white solid. LC-MS (ESI+): m/z 430 (MH+). ¹H NMR (300 MHz, CD₃OD) δ 8.36 (d, J=8.7 Hz, 1H), 7.74 (d, J=7.2 Hz, 2H), 7.52-7.43 (m, 3H), 6.95 (d, J=8.7 Hz, 1H), 6.47 (s, 1H), 4.31-4.25 (m, 4H), 3.92-3.87 (m, 4H).

### Example 6

### 4-Morpholino- N-(3-(pyridin-2-yl)-1H-pyrazol-5-yl)pyrido[3′,2′:4,5]furo[3,2-d]pyrimidin-2-amine hydrochloride.

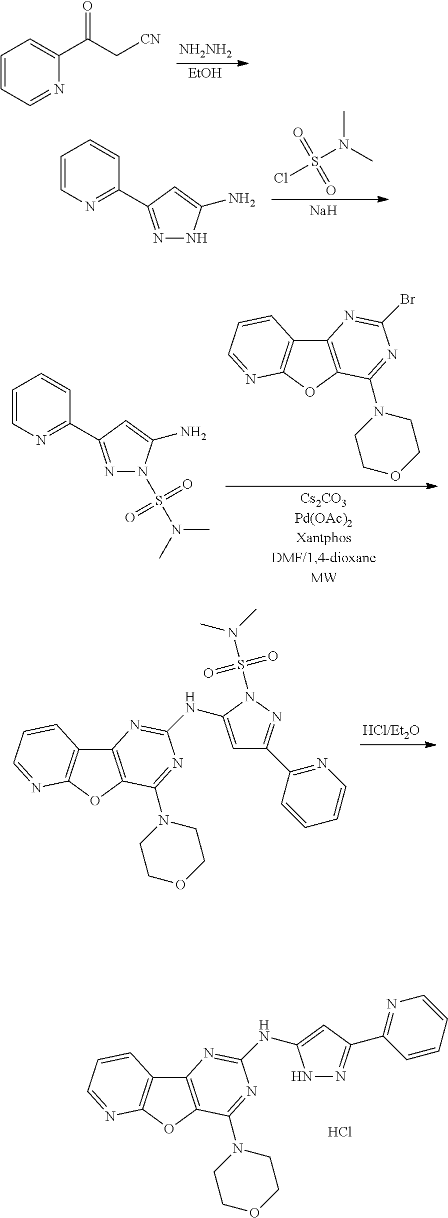

Step A: 3-(Pyridin-2-yl)-1H-pyrazol-5-amine. To a solution of 3-oxo-3-(pyridin-2-yl)propanenitrile (1 g, 6.84 mmol) and hydrazine (513 mg, 10.24 mmol, 99%) in EtOH (35 mL) was added two drops of AcOH. The reaction was heated to 80° C. for 5 h. The reaction mixture was concentrated directly under reduced pressure. The resulting residue was purified by silica gel column chromatography with a gradient elution of 20% EtOAc/PE to 33% EtOAc/PE to provide 3-(pyridin-2-yl)-1H-pyrazol-5-amine (630 mg, 3.9 mmol) as white solid. LC-MS (ESI+): m/z 161 (MH⁺). ¹H NMR (300 MHz, DMSO-d₆) δ 11.9 (brs, 1H), 8.53 (d, J=4.8 Hz, 1H), 7.78-7.76 (m, 2H), 7.26-7.22 (m, 1H), 5.93 (s, 1H), 4.82 (brs, 2H).

Step B: 5-Amino-N,N-dimethyl-3-(pyridin-2-yl)-1H-pyrazole-1-sulfonamide. To a solution of 3-(pyridin-2-yl)-1H-pyrazol-5-amine (630 mg, 3.94 mmol) in THF (5 mL) at 0° C. was added NaH (315 mg, 7.88 mmol). After stirring at 0° C. for 1 h, to the solution was added dimethylsulfamoyl chloride (676 mg, 4.72 mmol). The reaction mixture was quenched with saturated aq. NH₄Cl. The aqueous solution was extracted with ethyl acetate (3×50 mL). The combined organic phase was dried over anhydrous Na₂SO₄, filtrated, and concentrated under reduced pressure. The resulting residue was purified by silica gel column chromatography with a gradient elution of 20% EtOAc/PE to 33% EtOAc/PE to provide the title compound (310 mg, 1.16 mmol). LC-MS (ESI+): m/z 268 (MN). ¹H NMR (300 MHz, CDCl₃) δ 8.59 (d, J=5.7 Hz, 1H), 8.04 (d, J=8.1 Hz, 1H), 7.72 (td, J=7.8, 1.8 Hz, 1H), 7.25-7.22 (m, 1H), 6.11 (s, 1H), 4.89 (brs, 2H), 3.02 (s, 6H).

Step C: N,N-Dimethyl-5-((4-orpholinopyrido[3′,2′:4,5]furo[3,2-d]pyrimidin-2-yl)amino)-3-(pyridin-2-yl)-1H-pyrazole-1-sulfonamide. A solution of 2-bromo-4-morpholinopyrido[3′,2′:4,5]furo[3,2-d]pyrimidine (60 mg, 0.18 mmol), 5-amino-N,N-dimethyl-3-(pyridin-2-yl)-1H-pyrazole-1-sulfonamide (53 mg, 0.21 mmol), Cs₂CO₃ (146 mg, 0.45 mmol), Pd(OAc)₂ (2.4 mg, 0.01 mmol), and Xantphos (10 mg, 0.01 mmol) in DMF/1,4-dioxane (7/1, 2.4 mL) was heated to 90° C. for 40 min under microwave conditions. The reaction mixture was concentrated directly under reduced pressure. The resulting residue was purified by silica gel column chromatography with a gradient elution of 1% MeOH /DCM to 2% MeOH /DCM to provide the title compound (70 mg, 0.13 mmol) as a white solid. LC-MS (ESI+): m/z 522 (MH⁺) ⁱH NMR (300 MHz, CDCl₃) δ 8.80 (s, 1H), 8.71-8.69 (m, 1H), 8.59-8.54 (m, 2H), 8.09 (d, J=7.8 Hz, 1H), 7.61 (t, J=1.8 Hz, 1H), 7.64 (s, 1H), 7.46-7.42 (m, 1H), 7.32-7.25 (m, 1H), 4.17-4.14 (m, 4H), 3.91-3.88 (m, 4H), 3.09 (s, 6H).

Step D. To a solution of N,N-dimethyl-5-((4-orpholinopyrido[3′,2′:4,5]furo[3,2-d]pyrimidin-2-yl) amino)-3-(pyridin-2-yl)-1H-pyrazole-1-sulfonamide (70 mg, 0.13 mmol) in DCM (2 mL) was added a solution of HCl/Et₂O (2 mL). The reaction was stirred at rt for 2 h. The reaction mixture was concentrated directly and the resulting residue was slurried in MeOH/Et₂O (1/20, 5 mL) to provide the title compound (40.7 mg, 0.1 mmol) as a white solid. LC-MS (ESI+): m/z 415 (MH⁺). ¹H NMR (300 MHz, DMSO-d₆) δ 10.23 (s, 1H), 8.55 (d, J=7.5 Hz, 1H), 8.71 (d, J=5.1 Hz, 1H), 8.65 (d, J=3.3 Hz, 1H), 8.30-8.24 (m, 2H), 7.70-7.68 (m, 1H), 7.63-7.59 (m, 1H), 6.98 (s, 1H), 4.08-4.01 (m, 4H), 3.86-3.79 (m, 4H).

### Example 7

### N-(3-(4-Methylpyridin-2-yl)-1H-pyrazol-5-yl)-4-morpholinopyrido[3′,2′:4,5]furo[3,2-d]pyrimidin-2-amine hydrochloride.

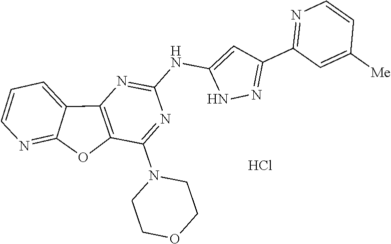 [SMILES: Cc1ccnc(-c2cc(Nc3nc(N4CCOCC4)c4oc5ncccc5c4n3)[nH]n2)c1 ; Cl]

Step A: 3-(4-Methylpyridin-2-yl)-3-oxopropanenitrile. To a solution of methyl 4-methylpicolinate (900 mg, 5.95 mmol) and anhydrous acetonitrile (367 mg, 8.93 mmol) in THF (45 mL) at 0° C. was added NaHMDS (4.5 mL, 8.9 mmol) dropwise. After addition, the reaction mixture was warmed to rt and stirred at rt for 0.5 h. The reaction mixture was quenched with saturated aq. NH₄Cl and extracted with EtOAc (3×20 mL). The combined organic phase was dried over anhydrous Na₂SO₄, filtrated, and concentrated. The residue was purified by silica column chromatography with a gradient elution of 20% EtOAc/PE to 33% EtOAc/PE to provide 3-(4-methylpyridin-2-yl)-3-oxopropanenitrile (780 mg, 4.87 mmol). ¹H NMR (300 MHz, CDCl₃) δ 8.53 (d, J=4.8 Hz, 1H), 7.93 (s, 1H), 7.37 (d, J=4.5 Hz, 1H), 4.37 (s, 2H), 2.46 (s, 3H).

Step B. The title compound was prepared as described in Example 6, using 3-(4-methylpyridin-2-yl)-3-oxopropanenitrile in Step A. LC-MS (ESI+): m/z 429 (MH+). ¹H NMR (300 MHz, DMSO-d₆) δ 10.22 (s, 1H), 8.85 (d, J=7.5 Hz, 1H), 8.65-8.59 (m, 2H), 8.24 (s, 1H), 7.69-7.59 (m, 2H), 6.96 (s, 1H), 4.04-4.01 (m, 4H), 3.82-3.79 (m, 4H), 2.61 (s, 3H).

### Example 8

### N-(3-Methyl-1H-pyrazol-5-yl)-4-morpholinopyrido[3′,2′:4,5]furo[3,2-d]pyrimidin-2-amine.

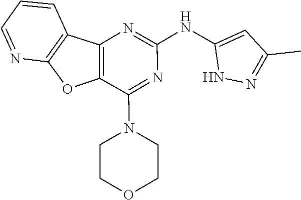 [SMILES: Cc1cc(Nc2nc(N3CCOCC3)c3oc4ncccc4c3n2)[nH]n1]

Trifluoracetic acid (25.8 mL, 337 mmol) was added to a mixture of 2-chloro-4-morpholinopyrido[3′,2′:4,5]furo[3,2-d]pyrimidine (WO2011/021038; 4.9 g, 16.9 mmol), 3-amino-5-methylpyrazole (2.80 g, 28.7 mmol), and iPrOH (25 mL) at 23° C. The mixture was heated and stirred at 120° C. for 12 h. The mixture was allowed to cool and was added to saturated aq. Na₂CO₃ (100 mL). The mixture was extracted with 1:1 THF:EtOAc (100 mL×2) and the combined organic phases were dried and concentrated. The residue was purified by preparatory HPLC (Xtimate C18 10μ 250 mm×50 mm column; water (10 mMNH₄HCO₃): ACN; B%: 20-50%) to give the titled compound. ¹H NMR (400 MHz CDCl₃): 6 8.47-8.52 (m, 1H), 8.33-8.38 (m, 1H), 7.33-7.38 (m, 1H), 5.97 (s, 1H), 3.99-4.07 (m, 4H), 3.78-3.84 (m, 4H), 2.25 (s, 3H). MS (ESI) 352.1 [ME]⁺.

### Example 9

### 4-Morpholino-N-(3-phenyl-1H-pyrazol-5-yl)pyrido[3′,2′:4,5]furo[3,2-d]pyrimidin-2-amine.

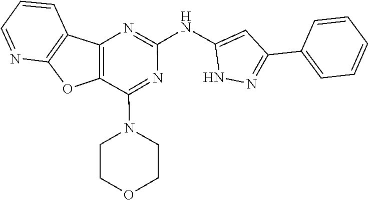 [SMILES: c1ccc(-c2cc(Nc3nc(N4CCOCC4)c4oc5ncccc5c4n3)[nH]n2)cc1]

The titled compound was prepared using 3-amino-5-phenylpyrazole in a manner analogous to Example 8. ¹H NMR (400 MHz CDCl₃): 6 8.59-8.64 (m, 1H), 8.44-8.50 (m, 1H), 7.79-7.84 (m, 2H), 7.44-7.47 (m, 3H), 7.27-7.42 (m, 2H), 6.30 (m, 1H), 4.08-4.26 (m, 4H), 3.87-3.95 (m, 4H). MS (ESI) 414.1 [ME]⁺.

### Example 10

### 4-Morpholino-N-(3-(pyridin-4-yl)-1H-pyrazol-5-yl)pyrido[3′,2′:4,5]furo[3,2-d]pyrimidin-2-amine.

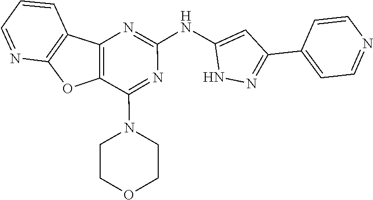 [SMILES: c1cnc2oc3c(N4CCOCC4)nc(Nc4cc(-c5ccncc5)n[nH]4)nc3c2c1]

The titled compound was prepared using 3-amino-5-(pyridin-4-yl)pyrazole in a manner analogous to Example 8. ¹H NMR (400 MHz, CDCl₃): 6 8.63-8.66 (m, 2H), 8.61-8.62 (m, 1H), 8.45-8.46 (m, 1H), 7.70-7.71 (m, 2H), 7.47-7.49 (m, 1H), 7.27-7.28 (m, 1H), 6.20 (s, 1H), 4.13-4.14 (m, 4H), 3.88-3.90 (m, 4H). MS (ESI) 415.1 [ME]⁺.

### Example 11

### N-(1,5-Dimethyl-1H-pyrazol-3-yl)-N-methyl-4-morpholinopyrido[3′,2′:4,5]furo[3,2-d]pyrimidin-2-amine.

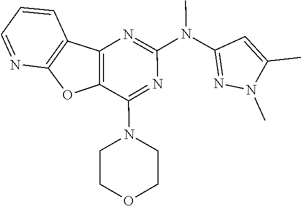 [SMILES: Cc1cc(N(C)c2nc(N3CCOCC3)c3oc4ncccc4c3n2)nn1C]

The titled compound was prepared using N,1,5-trimethyl-1H-pyrazol-3-amine in a manner analogous to Example 8. The crude product was chromatographed using silica gel (DCM:MeOH 100:0 to 96:4) to provide the purified compound. ¹H NMR (400 MHz, CDCl₃): δ 8.51 (dd, J=4.4, 0.7 Hz, 1H), 8.44 (d, J=7.4 Hz, 1H), 7.35 (dd, J=7.4, 5.1 Hz, 1H), 6.54 (s, 1H), 4.06 (t, J=4.5 Hz, 4H), 3.86 (t, J=4.5 Hz, 4H), 3.73 (s, 3H), 3.66 (s, 3H), 2.30 (s, 3H). MS (ESI) 380.2 [MH]⁺.

### Example 12

### N-(1-Methyl-1H-imidazol-2-yl)-4-morpholinopyrido[3′,2′:4,5]furo[3,2-d]pyrimidin-2-amine.

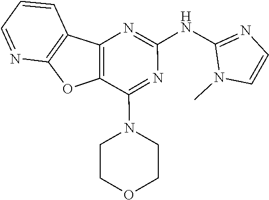 [SMILES: Cn1ccnc1Nc1nc(N2CCOCC2)c2oc3ncccc3c2n1]

2-Chloro-4-morpholinopyrido[3′,2′:4,5]furo[3,2-d]pyrimidine (75 mg, 0.26 mmol), 1-methyl-1H-imidazol-2-amine hydrochloride (41 mg, 0.31 mmol), cesium carbonate (252 mg, 0.774 mmol), palladium acetate (15 mg, 0.064 mmol), rac-BINAP (40 mg, 0.064 mmol), and DMF (1.3 mL) were combined in a sealed tube under argon and stirred at 200° C. for 3 h. The reaction mixture allowed to cool and was then chromatographed (C18 SiO₂ (10:90 to 100:0 ACN/H₂O with 0.1% TFA)) to provide the titled compound. ¹H NMR (400 MHz, DMSO): δ 8.61 (dd, J=4.8, 1.7 Hz, 1H); 8.43-8.45 (m, 1H), 7.53 (dd, J=7.6, 4.8 Hz, 1H), 7.10 (s, 1H), 6.79 (s, 1H), 3.91 (dd, J=5.7, 3.6 Hz, 4H), 3.76 (t, J=4.6 Hz, 4 H), 3.48 (s, 3H). MS (ESI) 352.2 [MH]⁺.

### Example 13

### 8-(2-(Dimethylamino)ethyl)-4-morpholino-N-(3-phenyl-1H-pyrazol-5-yl)pyrido[3′,2′:4,5]furo[3,2-d]pyrimidin-2-amine.

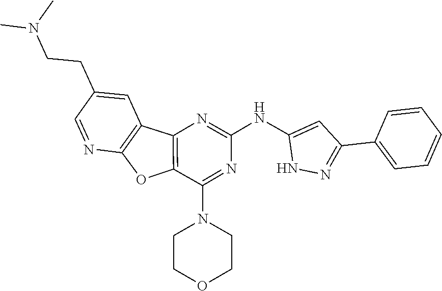 [SMILES: CN(C)CCc1cnc2oc3c(N4CCOCC4)nc(Nc4cc(-c5ccccc5)n[nH]4)nc3c2c1]

Step A: (E)-2-Chloro-8-(2-ethoxyvinyl)-4-morpholinopyrido[3′,2′:4,5]furo[3,2-d]pyrimidine. A mixture of 8-bromo-2-chloro-4-morpholinopyrido[3′,2′:4,5]furo[3,2-d]pyrimidine (CAS #1268241-78-6 (see WO2017/029514, WO2017/029521, WO2017/029519, WO2015/121657); 500 mg, 1.35 mmol), tetrakis(triphenylphosphine)palladium (156 mg, 0.135 mmol), (E)-2-(2-ethoxyvinyl)-4,4,5,5-tetramethyl-1,3,2-dioxaborolane (402 mg, 0.43 mmol), Na₂CO₃ (315 mg, 3.0 mmol), DME (8 mL), and H₂O (2 mL) was heated to 75° C. with stirring for 12 h. The mixture was allowed to cool to rt and was extracted with DCM (2×30 mL). The combined organic phases were washed sequentially with water and brine, then dried (Na₂SO₄) and concentrated. The residue was chromatographed (SiO₂, 0:100 to 30:70 EtOAc/hexanes) to provide the titled compound. MS (ESI) 414.1[MH]⁺.

Step B. A mixture of (E)-2-chloro-8-(2-ethoxyvinyl)-4-morpholinopyrido[3′,2′:4,5]furo[3,2-d]pyrimidine (290 mg, 0.80 mmol), THF (10 mL), and 4 M HCl (2.75 mL) was heated to reflux for 1.5 h. The reaction mixture was allowed to cool to rt and was poured into saturated aqueous NaHCO₃ (20 mL). The mixture was extracted with EtOAc (3×) and the organic phases were combined, washed with brine, then dried (Na₂SO₄) and concentrated. This material (270 mg) was taken up in DCM (5 mL) and a 2 M THF solution of dimethylamine (0.53 mL, 1.06 mmol) was added. The solution was maintained at rt for 20 min, then sodium triacetoxyborohydride (224 mg, 1.06 mmol) was added and the mixture was stirred for 90 min at rt. The mixture was poured into saturated NaHCO₃ and extracted with DCM (2×). The combined organic phases were dried (Na₂SO₄) and concentrated. This residue (290 mg) was combined with 3-amino-5-phenylpyrazole (153 mg, 0.96 mmol), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhos Pd G3, 91 mg, 0.10 mmol), dicyclohexyl(2′,4′,6′-triisopropyl-3,6-dimethoxy-[1,1′-biphenyl]-2-yl)phosphine (54 mg, 0.10 mmol), sodium tert-butoxide (92 mg, 0.96 mmol), and dioxane (4 mL) under argon in a dry reaction tube. The mixture was sparged with argon, then sealed and heated with stirring to 100° C. for 18 h. The mixture was allowed to cool to rt, then was diluted with DCM and washed with water. The aqueous layer was separated and extracted with DCM, then the combined organic phases were dried (Na₂SO₄) and concentrated. The residue was chromatographed (SiO₂, 0:100 to 30:70 MeOH/DCM) to provide the titled compound. ¹H NMR (400 MHz, DMSO): 6 12.59 (br s, 1H), 8.44 (s, 1H), 7.76 (d, J=7.6 Hz, 2H), 7.42 (s, 2H), 3.99 (s, 4H), 3.78 (s, 4H), 2.91 (t, J=7.3 Hz, 2H), 2.57 (t, J=7.3 Hz, 2H), 2.21 (s, 6H). MS (ESI) 485.3 [MH]⁺.

### Example 14

### 4-Morpholino-N-(3-phenyl-1H-pyrazol-5-yl)-8-(2-(pyrrolidin-1-yl)ethyl)pyrido[3′,2′:4,5]furo[3,2-d]pyrimidin-2-amine.

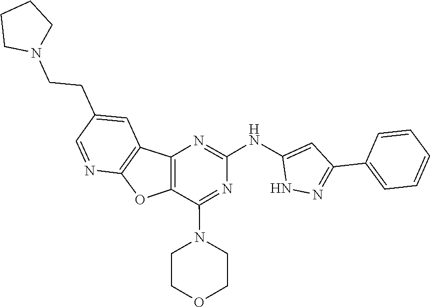 [SMILES: c1ccc(-c2cc(Nc3nc(N4CCOCC4)c4oc5ncc(CCN6CCCC6)cc5c4n3)[nH]n2)cc1]

The titled compound was prepared in a manner analogous to Example 13, using pyrrolidine in place of dimethylamine in Step B. ¹H NMR (400 MHz, DMSO): δ 8.45 (d, J=2.1 Hz, 1H), 7.76 (d, J=7.7 Hz, 2H), 7.42 (t, J=7.5 Hz, 2H), 7.31 (d, J=7.6 Hz, 1H), 3.99 (t, J=4.6 Hz, 4H), 3.78 (t, J=4.6 Hz, 4H), 2.95 (t, J=7.3 Hz, 2 H), 2.75 (t, J=7.3 Hz, 2H), 2.53 (s, 4H), 1.68 (s, 4H). MS (ESI) 511.4 [MH]⁺.

The compounds in Table 2 were prepared using methods analogous to those described in the reference example method.

TABLE 2

| | | | *Ex. | |
|---|---|---|---|---|
| Ex. | Chemical Name | Structure | Method | Data |
| 15 | N-(3-(2- fluorophenyl)-1H- pyrazol-5-yl)-4- morpholinopyrido [3′,2′:4,5]furo[3,2- d]pyrimidin-2- amine hydrochloride | 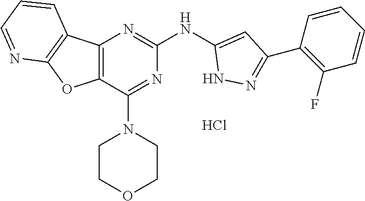 [SMILES: Fc1ccccc1-c1cc(Nc2nc(N3CCOCC3)c3oc4ncccc4c3n2)[nH]n1 ; Cl] | 1 | LC-MS: m/z 432 (MH⁺). ¹H NMR (300 MHz, DMSO-d₆) δ 10.02 (s, 1H), 8.72 (d, J = 7.5 Hz, 1H), 8.65 (d, J = 4.5 Hz, 1H), 7.92 (t, J = 3.9 Hz, 1H), 7.62-7.57 (m, 1H), 7.42-7.28 (m, 3H), 6.73 (s, 1H), 4.12-4.06 (m, 4H), 3.87-3.80 (m, 4H). |
| 16 | N-(3-(3- fluorophenyl)-1H- pyrazol-5-yl)-4- morpholinopyrido [3′,2′:4,5]furo[3,2- d]pyrimidin-2- amine hydrochloride | 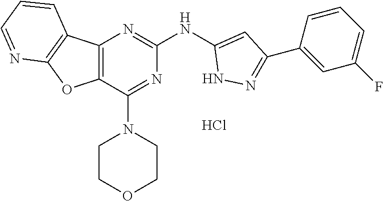 [SMILES: Fc1cccc(-c2cc(Nc3nc(N4CCOCC4)c4oc5ncccc5c4n3)[nH]n2)c1 ; Cl] | 1 | LC-MS: m/z 432 (MH⁺). ¹H NMR (300 MHz, DMSO-d₆) δ 9.98 (s, 1H), 8.82 (d, J = 6.6 Hz, 1H), 8.64 (d, J = 3.3 Hz, 1H), 7.66-7.57 (m, 3H), 7.50- 7.46 (m, 1H), 7.17 (t, J = 7.2 Hz, 1H), 6.72 (s, 1H), 4.11-4.06 (m, 4H), 3.88- 3.82 (m, 4H). |
| 17 | N-(3-(3- chlorophenyl)-1H- pyrazol-5-yl)-4- morpholinopyrido [3′,2′:4,5]furo[3,2- d]pyrimidin-2- amine hydrochloride | 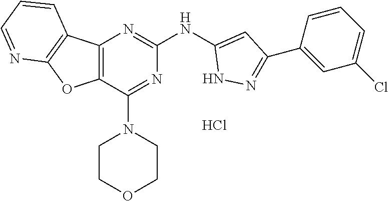 [SMILES: Clc1cccc(-c2cc(Nc3nc(N4CCOCC4)c4oc5ncccc5c4n3)[nH]n2)c1 ; Cl] | 1 | LC-MS: m/z 448 (MH⁺). ¹H NMR (300 MHz, CD₃OD) δ 8.61 (d, J = 4.8 Hz, 1H), 8.54 (dd, J = 7.8, 1.5 Hz, 1H), 7.68 (s, 1H), 7.59-7.55 (m, 2H), 7.42- 7.33 (m, 2H), 6.44 (s, 1H), 4.25-4.18 (m, 4H), 3.85- 3.82 (m, 4H). |
| 18 | 4-morpholino-N- (3-(3- (trifluoromethyl) phenyl)-1H- pyrazol-5- yl)pyrido[3′,2′:4,5] furo[3,2- d]pyrimidin-2- amine hydrochloride | 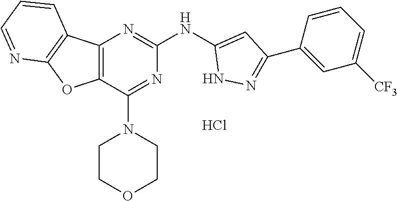 [SMILES: FC(F)(F)c1cccc(-c2cc(Nc3nc(N4CCOCC4)c4oc5ncccc5c4n3)[nH]n2)c1 ; Cl] | 1 | LC-MS: m/z 482 (MH⁺). ¹H NMR (300 MHz, DMSO-d₆) δ 9.97 (s, 1H), 8.81 (d, J = 7.5 Hz, 1H), 8.64 (dd, J = 4.8, 1.8 Hz, 1H), 8.13-8.09 (m, 2H), 7.69 (d, J = 4.8 Hz, 2H), 8.61 (dd, J = 7.5, 4.8 Hz, 1H), 6.73 (s, 1H), 4.09- 4.04 (m, 4H), 3.84-3.81 (m, 4H). |
| 19 | N-(3-(3,4- difluorophenyl)- 1H-pyrazol-5-yl)- 4- morpholinopyrido [3′,2′:4,5]furo[3,2- d]pyrimidin-2- amine hydrochloride | 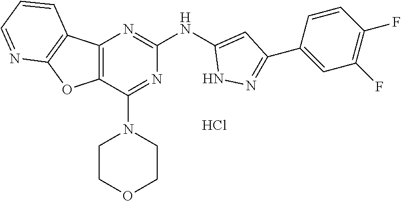 [SMILES: Fc1ccc(-c2cc(Nc3nc(N4CCOCC4)c4oc5ncccc5c4n3)[nH]n2)cc1F ; Cl] | 1 | LC-MS: m/z 450 (MH⁺). ¹H NMR (300 MHz, DMSO-d₆) δ 9.99 (s, 1H), 8.82 (d, J = 6.6 Hz, 1H), 8.64 (dd, J = 4.8, 1.5 Hz, 1H), 7.88-7.80 (m, 1H), 7.61-7.46 (m, 3H), 6.66 (s, 1H), 4.07-4.01 (m, 4H), 3.83-3.76 (m, 4H). |
| 20 | 4-morpholino-N- (4-phenyl-1H- imidazol-2- yl)pyrido[3′,2′:4,5] furo[3,2- d]pyrimidin-2- amine hydrochloride | 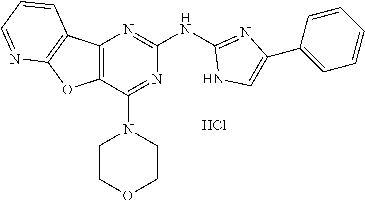 [SMILES: c1ccc(-c2c[nH]c(Nc3nc(N4CCOCC4)c4oc5ncccc5c4n3)n2)cc1 ; Cl] | 1 | LC-MS: m/z 414 (MH⁺). ¹H NMR (300 MHz, DMSO-d₆) δ 8.82 (dd, J = 7.8, 1.8 Hz, 1H), 8.68 (dd, J = 4.8, 1.8 Hz, 1H), 7.80 (d, J = 7.5Hz, 2H), 7.72 (s, 1H), 7.66 (dd, J = 7.5, 4.8 Hz, 1H), 7.54-7.49 (m, 2H), 7.43-7.40 (m, 1H), 4.12-4.06 (m, 4H), 3.87- 3.81 (m, 4H). |
| 21 | 4-morpholino-N- phenethylpyrido [3′,2′:4,5]furo[3,2- d]pyrimidin-2- amine | 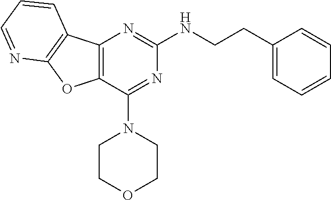 [SMILES: c1ccc(CCNc2nc(N3CCOCC3)c3oc4ncccc4c3n2)cc1] | 2 | LC-MS (ESI+): m/z 376 (MH⁺). ¹H NMR (300 MHz, CDCl₃) δ 8.53 (dd, J = 4.8, 1.8 Hz, 1H), 8.44 (dd, J = 7.8, 1.8 Hz, 1H), 7.39-7.20 (m, 6H), 4.11- 4.07 (m, 4H), 3.87-3.84 (m, 4H), 3.75-3.68 (m, 2H), 2.95 (t, J = 7.2 Hz, 2H). |
| 22 | phenyl(4- morpholinopyrido [3′,2′:4,5]furo[3,2- d]pyrimidin-2- yl)carbamate hydrochloride | 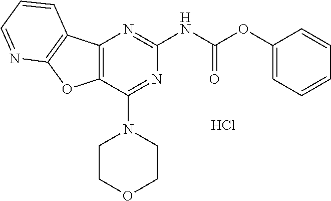 [SMILES: O=C(Nc1nc(N2CCOCC2)c2oc3ncccc3c2n1)Oc1ccccc1 ; Cl] | 3 | LC-MS: m/z 392 (MH⁺). ¹H NMR (300 MHz, CDCl₃) δ 10.00 (s, 1H), 8.78-8.71 (m, 2H), 7.60- 7.54 (m, 1H), 7.44-7.39 (m, 2H), 7.30-7.20 (m, 3H), 4.40-4.25 (m, 4H), 3.90-3.77 (m, 4H). |
| 23 | 7-(3- (dimethylamino) propoxy)-4- morpholino-N-(3- phenyl-1H- pyrazol-5- yl)pyrido[3′,2′:4,5] furo[3,2- d]pyrimidin-2- amine hydrochloride | 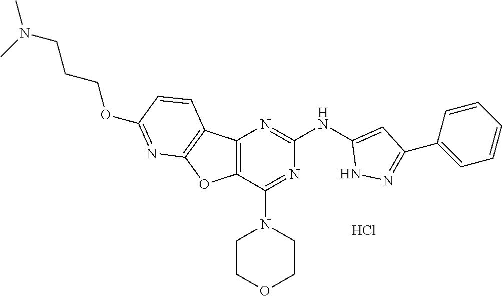 [SMILES: CN(C)CCCOc1ccc2c(n1)oc1c(N3CCOCC3)nc(Nc3cc(-c4ccccc4)n[nH]3)nc12 ; Cl] | 4 | LC-MS (ESI+): m/z 515 (MH⁺). ¹H NMR (300 MHz, CD₃OD) δ 8.48 (d, J = 8.7 Hz, 1H), 7.73 (d, J = 6.9 Hz, 2H), 7.53-7.44 (m, 3H), 7.12 (d, J = 8.4 Hz, 1H), 6.48 (s, 1H), 4.58 (t, J = 5.7 Hz, 2H), 4.31- 4.24 (m, 4H), 3.95-3.88 (m, 4H), 3.45-3.36 (m, 2H), 2.96 (s, 6H), 2.36- 2.29 (m, 2H). |
| 24 | 4-morpholino-7- (2- morpholinoethoxy)- N-(3-phenyl-1H- pyrazol-5- yl)pyrido[3′,2′:4,5] furo[3,2- d]pyrimidin-2- amine hydrochloride | 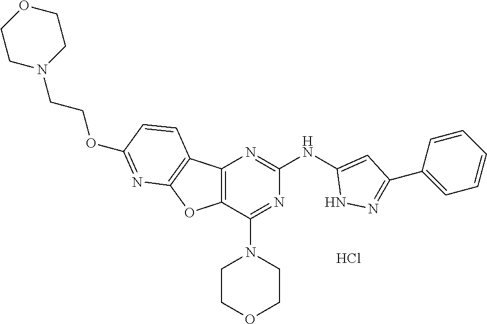 [SMILES: c1ccc(-c2cc(Nc3nc(N4CCOCC4)c4oc5nc(OCCN6CCOCC6)ccc5c4n3)[nH]n2)cc1 ; Cl] | 4 | LC-MS (ESI+): m/z 543 (MH⁺). ¹H NMR (300 MHz, CD₃OD) δ 8.54 (d, J = 8.7 Hz, 1H), 7.74 (dd, J = 8.4, 1.5 Hz, 2H), 7.56- 7.44 (m, 3H), 7.22 (d, J = 8.4 Hz, 1H), 6.49 (s, 1H), 4.32-4.25 (m, 4H), 4.11- 4.07 (m, 2H), 3.93-3.83 (m, 6H), 3.76-3.73 (m, 2H), 3.68-3.64 (m, 3H), 3.47-3.32 (m, 3H). |

| | | | | |
|---|---|---|---|---|
| 25 | 4-morpholino-7- (3- morpholinopropoxy)- N-(3-phenyl- 1H-pyrazol-5- yl)pyrido[3′,2′:4,5] furo[3,2- d]pyrimidin-2- amine hydrochloride | 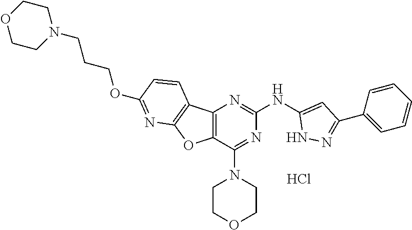 [SMILES: c1ccc(-c2cc(Nc3nc(N4CCOCC4)c4oc5nc(OCCCN6CCOCC6)ccc5c4n3)[nH]n2)cc1 ; Cl] | 4 | LC-MS (ESI+): m/z 557 (MH⁺). ¹H NMR (300 MHz, DMSO-d₆ + D₂O) δ 8.61 (d, J = 8.4 Hz, 1H), 7.80 (d, J = 7.2 Hz, 2H), 7.50-7.46 (m, 2H), 7.40- 7.38 (m, 1H), 7.06 (d, J = 8.4 Hz, 1H), 6.69 (s, 1H), 4.47 (t, J = 6.0 Hz, 2H), 4.12-4.07 (m, 4H), 4.00- 3.96 (m, 2H), 3.83-3.75 (m, 6H), 3.30-3.27 (m, 4H), 3.13-3.06 (m, 2H), 2.31-2.19 (m, 2H). |

| | | | | |
|---|---|---|---|---|
| 26 | 4-morpholino-N- (3-phenyl-1H- pyrazol-5-yl)-7- (2-(pyrrolidin-1- yl)ethoxy)pyrido [3′,2′:4,5]furo[3,2- d]pyrimidin-2- amine hydrochloride | 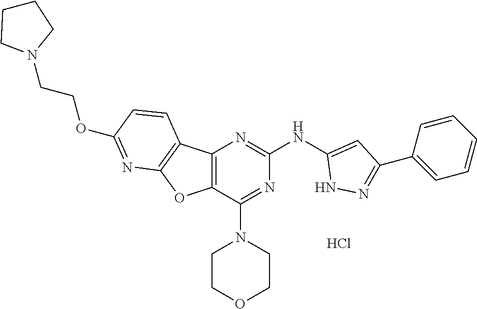 [SMILES: c1ccc(-c2cc(Nc3nc(N4CCOCC4)c4oc5nc(OCCN6CCCC6)ccc5c4n3)[nH]n2)cc1 ; Cl] | 4 | LC-MS (ESI+): m/z 527 (MH⁺). ¹H NMR (300 MHz, DMSO-d₆ + D₂O) δ 8.65 (d, J = 8.4 Hz, 1H), 8.15 (d, J = 8.4 Hz, 2H), 7.48-7.45 (m, 2H), 7.40- 7.35 (m, 1H), 7.13 (d, J = 8.4 Hz, 1H), 6.71 (s, 1H), 4.72 (t, J = 6.0 Hz, 2H), 4.11-4.05 (m, 4H), 3.85- 3.83 (m, 4H), 3.66-3.58 (m, 4H), 3.18-3.08 (m, 2H), 2.08-1.86 (m, 4H). |
| 27 | 4-morpholino-N- (3-phenyl-1H- pyrazol-5-yl)-7- (3-(pyrrolidin-1- yl)propoxy)pyrido [3′,2′:4,5]furo[3,2- d]pyrimidin-2- amine hydrochloride | 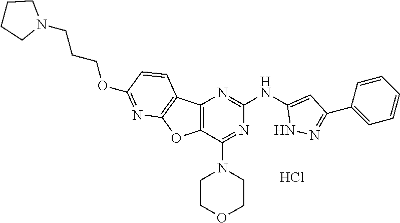 [SMILES: c1ccc(-c2cc(Nc3nc(N4CCOCC4)c4oc5nc(OCCCN6CCCC6)ccc5c4n3)[nH]n2)cc1 ; Cl] | 4 | LC-MS (ESI+): m/z 541 (MH⁺). ¹H NMR(300 MHz, DMSO-d₆) δ 8.61 (d, J = 8.7 Hz, 1H), 7.79 (d, J = 7.2 Hz, 2H), 7.49- 7.44 (m, 2H), 7.38-7.34 (m, 1H), 7.03 (d, J = 8.4 Hz, 1H), 6.72 (s, 1H), 4.46 (t, J = 6.0 Hz, 2H), 4.08- 4.03 (m, 4H), 3.83-3.79 (m, 4H), 3.58-3.54 (m, 2H), 3.32-3.29 (m, 2H), 3.05-3.02 (m, 2H), 2.22- 2.17 (m, 2H), 2.03-2.01 (m, 2H), 1.90-1.86 (m, 2H). |
| 28 | 7-(2-(4- methylpiperazin- 1-yl)ethoxy)-4- morpholino-N-(3- phenyl-1H- pyrazol-5- yl)pyrido[3′,2′:4,5] furo[3,2- d]pyrimidin-2- amine hydrochloride | 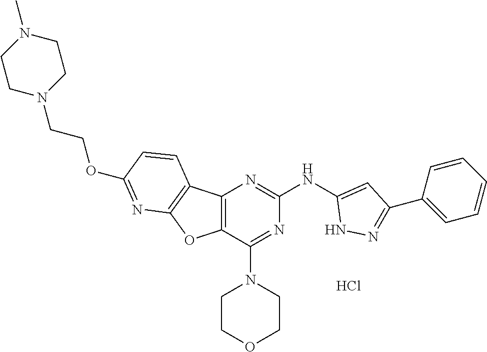 [SMILES: CN1CCN(CCOc2ccc3c(n2)oc2c(N4CCOCC4)nc(Nc4cc(-c5ccccc5)n[nH]4)nc23)CC1 ; Cl] | 4 | LC-MS (ESI+): m/z 556 (MH⁺). ¹H NMR (300 MHz, DMSO-d₆ + D₂O) δ 8.64 (d, J = 8.7 Hz, 1H), 7.80 (d, J = 7.8 Hz, 2H), 7.48-7.44 (m, 2H), 7.37- 7.32 (m, 1H), 7.08 (d, J = 8.7 Hz, 1H), 6.71 (s, 1H), 4.73 (t, J = 6.0 Hz, 2H), 4.07-4.01 (m, 4H), 3.85- 3.79 (m, 4H), 3.72-3.53 (m, 8H), 2.83-2.79 (m, 5H). |
| 29 | 7-(3-(4- methylpiperazin- 1-yl)propoxy)-4- morpholino-N-(3- phenyl-1H- pyrazol-5- yl)pyrido[3′,2′:4,5] furo[3,2- d]pyrimidin-2- amine hydrochloride | 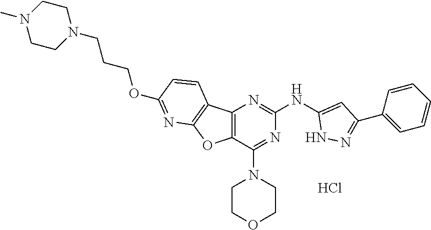 [SMILES: CN1CCN(CCCOc2ccc3c(n2)oc2c(N4CCOCC4)nc(Nc4cc(-c5ccccc5)n[nH]4)nc23)CC1 ; Cl] | 4 | LC-MS (ESI+): m/z 570 (MH⁺). ¹H NMR (300 MHz, CD₃OD) δ 8.47 (d, J = 8.7 Hz, 1H), 7.74 (d, J = 6.9 Hz, 2H), 7.52-7.41 (m, 3H), 7.12 (d, J = 8.7 Hz, 1H), 6.48 (s, 1H), 4.62 (t, J = 6.0 Hz, 2H ), 4.31-4.25 (m, 4H), 3.95-3.91 (m, 4H), 3.87-3.84 (m, 6H), 3.63-3.59 (m, 4H), 3.03 (s, 3H), 2.45-2.37 (m, 2H). |
| 30 | 7-methoxy-4- morpholino-N-(3- phenyl-1H- pyrazol-5- yl)pyrido[3′,2′:4,5] furo[3,2- d]pyrimidin-2- amine hydrochloride | 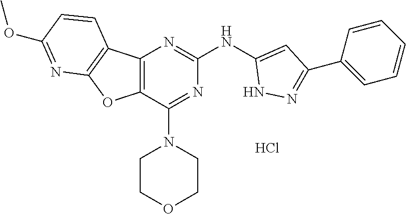 [SMILES: COc1ccc2c(n1)oc1c(N3CCOCC3)nc(Nc3cc(-c4ccccc4)n[nH]3)nc12 ; Cl] | 4 | LC-MS (ESI+): m/z 444 (MH⁺). ¹H NMR (300 MHz, CD₃OD) δ 8.41 (d, J = 8.7 Hz, 1H), 7.73 (d, J = 6.9 Hz, 2H), 7.52-7.41 (m, 3H), 7.07 (d, J = 8.7 Hz, 1H), 6.46 (s, 1H), 4.31- 4.23 (m, 4H), 4.07 (s, 3H), 3.93-3.88 (m, 4H). |
| 31 | 4-morpholino-N- (3-(pyridin-3-yl)- 1H-pyrazol-5- yl)pyrido[3′,2′:4,5] furo[3,2- d]pyrimidin-2- amine hydrochloride | 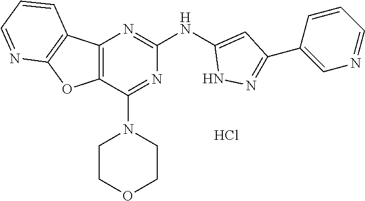 [SMILES: c1cncc(-c2cc(Nc3nc(N4CCOCC4)c4oc5ncccc5c4n3)[nH]n2)c1 ; Cl] | 6 | LC-MS (ESI+): m/z 415 (MH⁺). ¹H NMR (300 MHz, DMSO-d₆) δ 10.16 (s, 1H), 9.24 (s, 1H), 8.89 (d, J = 7.5 Hz, 1H), 8.78 (d, J = 5.1 Hz, 2H), 8.64 (d, J = 3.6 Hz, 1H), 8.02- 7.98 (m, 1H), 7.62-7.58 (m, 1H), 6.76 (s, 1H), 4.09-4.01 (m, 4H), 3.86- 3.79 (m, 4H). |
| 32 | N-(3-(5- methylpyridin-3- yl)-1H-pyrazol-5- yl)-4- morpholinopyrido [3′,2′:4,5]furo[3,2- d]pyrimidin-2- amine hydrochloride | 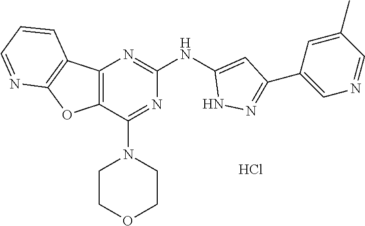 [SMILES: Cc1cncc(-c2cc(Nc3nc(N4CCOCC4)c4oc5ncccc5c4n3)[nH]n2)c1 ; Cl] | 7 | LC-MS (ESI+): m/z 429 (MH⁺). ¹H NMR (300 MHz, DMSO-d₆) δ 10.18 (s, 1H), 9.11 (s, 1H), 8.88 (d, J = 7.2 Hz, 1H), 8.75 (s, 1H), 8.71 (s, 1H), 8.65- 8.63 (m, 1H), 7.62-7.58 (m, 1H), 6.76 (s, 1H), 4.09-4.01 (m, 4H), 3.82- 3.79 (m, 4H), 2.55 (s, 3H). |
| 33 | N-(3-(6- methylpyridin-2- yl)-1H-pyrazol-5- yl)-4- morpholinopyrido [3′,2′:4,5]furo[3,2- d]pyrimidin-2- amine hydrochloride | 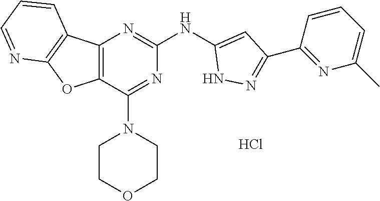 [SMILES: Cc1cccc(-c2cc(Nc3nc(N4CCOCC4)c4oc5ncccc5c4n3)[nH]n2)n1 ; Cl] | 7 | LC-MS (ESI+): m/z 429 (MH⁺) ¹H NMR (300 MHz, DMSO-d₆) δ 10.26 (s, 1H), 8.83 (d, J = 7.5 Hz, 1H), 8.65 (dd, J = 4.8, 1.5 Hz, 1H), 8.32-8.26 (m, 1H), 8.14 (d, J = 7.8 Hz, 1H), 7.66-7.59 (m, 2H), 7.02 (s, 1H), 4.09-4.01 (m, 4H), 3.86-3.79 (m, 4H), 2.75 (s, 3H). |
| 34 | N-(3-(3- methoxyphenyl) - 1H-pyrazol-5-yl)- 4- morpholinopyrido [3′,2′:4,5]furo[3,2- d]pyrimidin-2- amine hydrochloride | 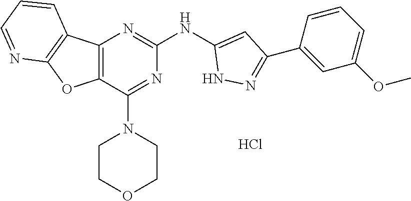 [SMILES: COc1cccc(-c2cc(Nc3nc(N4CCOCC4)c4oc5ncccc5c4n3)[nH]n2)c1 ; Cl] | 7 | LC-MS (ESI+): m/z 444 (MH⁺). ¹H NMR (300 MHz, CD₃OD) δ 8.72- 8.70 (m, 1H), 8.65 (dd, J = 4.8, 1.5 Hz, 1H), 7.67 (dd, J = 8.1, 4.8 Hz, 1H), 7.44- 7.39 (m, 1H), 7.30 (d, J = 7.8 Hz, 2H), 7.02 (d, J = 8.1 Hz, 1H), 6.49 (s, 1H), 4.39-4.31 (m, 4H), 3.94- 3.91 (m, 4H), 3.87 (s, 3H). |
| 35 | 4-morpholino-N- (3-(3- (trifluoromethoxy) phenyl)-1H- pyrazol-5- yl)pyrido[3′,2′:4,5] furo[3,2- d]pyrimidin-2- amine hydrochloride | 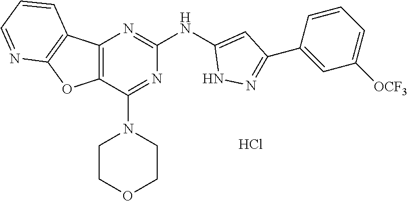 [SMILES: FC(F)(F)Oc1cccc(-c2cc(Nc3nc(N4CCOCC4)c4oc5ncccc5c4n3)[nH]n2)c1 ; Cl] | 7 | LC-MS (ESI+): m/z 498 (MH⁺). ¹H NMR (300 MHz, CD₃OD) δ 8.73- 8.71 (m, 1H), 8.64 (dd, J = 7.8, 1.8 Hz, 1H), 7.75 (d, J = 8.1 Hz, 1H), 7.69-7.59 (m, 3H), 7.36 (d, J = 8.4 Hz, 1H), 6.58 (s, 1H), 4.31-4.28 (m, 4H), 3.94- 3.91 (m, 4H). |
| 36 | (S)-N-(1- cyclopropylethyl)- 4- morpholinopyrido [3′,2′:4,5]furo[3,2- d]pyrimidin-2- amine | 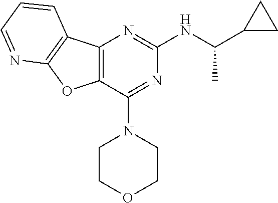 [SMILES: C[C@H](Nc1nc(N2CCOCC2)c2oc3ncccc3c2n1)C1CC1] | 2 | ¹H NMR (CDCl₃) δ 8.26 (d, 1H), 8.20 (d, 1H), 7.12 (dd, 1H), 3.80 (br s, 4H), 3.59 (d, 4H), 3.25 (m, 1H), 1.04 S (d, 3H), 0.35 (m, 1H), 0.0-0.25 (m, 3H); LCMS (M + H): 341 m/z. |
| 37 | 4-morpholino-N- phenylpyrido[3′,2′: 4,5]furo[3,2- d]pyrimidin-2- amine | 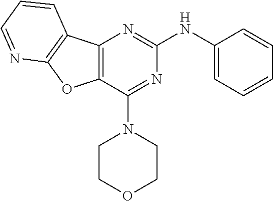 [SMILES: c1ccc(Nc2nc(N3CCOCC3)c3oc4ncccc4c3n2)cc1] | 12 | ¹H NMR (CDCl₃) δ 8.56 (d, 1H), 8.5 (d, 1H), 7.64 (d, 2H), 7.41 (m, 1H), 7.35 (m, 2H), 7.04 (m, 1H), 4.11 (br s, 4H), 3.88 (dd, 4H); LCMS (M + H): 348.15 m/z. |
| 38 | 4-morpholino-N- (pyridin-4- yl)pyrido[3′,2′:4,5] furo[3,2- d]pyrimidin-2- amine | 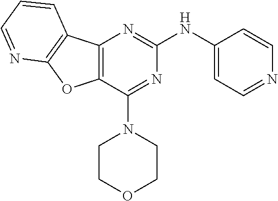 [SMILES: c1cnc2oc3c(N4CCOCC4)nc(Nc4ccncc4)nc3c2c1] | 12 | ¹H NMR (CDCl₃) δ 9.4 (s, 1H), 9.12 (d, 2H), 8.68 (s, 1H), 8.54 (s, 1H), 7.55 (m, 1H), 7.45 (d, 2H), 4.1 (broad, 4H), 3.94 (d, 4H). |
| 39 | 4-morpholino-N- (pyridin-3- yl)pyrido[3′,2′:4,5] furo[3,2- d]pyrimidin-2- amine | 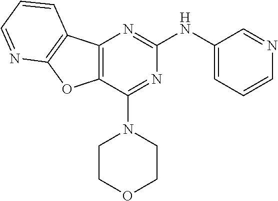 [SMILES: c1cncc(Nc2nc(N3CCOCC3)c3oc4ncccc4c3n2)c1] | 12 | ¹H NMR (DMSO-d₆) δ 9.54 (s, 1H), 8.93 (d, 1H), 8.63 (dd, 1H), 8.5 (d, 1H), 8.28 (d, 1H), 8.12 (d, 1H), 7.58 (dd, 1H), 7.33 (dd, 1H), 4.02 (d, 4H), 3.82 (dd, 4H); LCMS (M + H): 349.1 m/z. |
| 40 | 4-morpholino-N- (pyridin-2- yl)pyrido[3′,2′:4,5] furo[3,2- d]pyrimidin-2- amine | 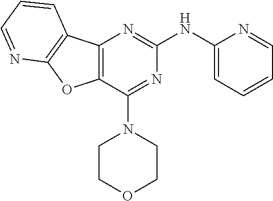 [SMILES: c1ccc(Nc2nc(N3CCOCC3)c3oc4ncccc4c3n2)nc1] | 12 | ¹H NMR (DMSO-d₆) δ 9.33 (s, 1H), 8.63 (d, 1H), 8.50 (dd, 1H), 8.39 (d, 1H), 7.78 (dd, 1H), 7.58 (dd, 1H), 6.97 (dd, 1H), 4.03 (d, 4H), 3.82 (dd, 4H); LCMS (M + H): 349.1 m/z. |
| 41 | 4-morpholino-N- (pyrimidin-4- yl)pyrido[3′,2′:4,5] furo[3,2- d]pyrimidin-2- amine | 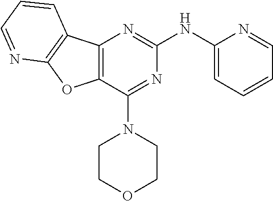 [SMILES: c1ccc(Nc2nc(N3CCOCC3)c3oc4ncccc4c3n2)nc1] | 12 | ¹H NMR (DMSO-d₆) δ 10.1 (broad, 1H), 8.74 (s, 1H), 8.64-8.35 (m, 4H), 7.61 (dd, 1H), 4.05 (m, 4H), 3.82 (m, 4H); LCMS (M + H): 350.1 m/z. |
| 42 | 4-morpholino-N- (pyrimidin-2- yl)pyrido[3′,2′:4,5] furo[3,2- d]pyrimidin-2- amine | 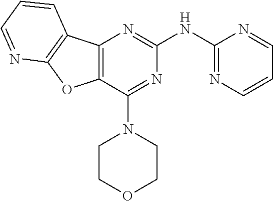 [SMILES: c1cnc(Nc2nc(N3CCOCC3)c3oc4ncccc4c3n2)nc1] | 12 | ¹H NMR (DMSO-d₆) δ 9.95 (s, 1H), 8.65-8.35 (m, 4H), 7.58 (dd, 1H), 7.0 (dd, 1H), 4.02 (m, 4H), 3.79 (m, 4H); LCMS (M + H): 350.1 m/z. |
| 43 | 4-morpholino-N- (pyrimidin-5- yl)pyrido[3′,2′:4,5] furo[3,2- d]pyrimidin-2- amine | 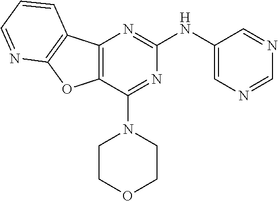 [SMILES: c1cnc2oc3c(N4CCOCC4)nc(Nc4cncnc4)nc3c2c1] | 12 | ¹H NMR (DMSO-d₆) δ 9.70 (s, 1H), 9.22 (s, 2H), 8.74 (s, 1H), 8.56 (dd, 1H), 7.58 (t, 1H), 4.03 (m, 4H), 3.83 (m, 4H); LCMS (M + H): 350.1 m/z. |
| 44 | N-([1,1′- biphenyl]-4-yl)-4- morpholinopyrido [3′,2′:4,5]furo[3,2- d]pyrimidin-2- amine | 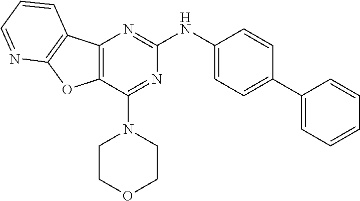 [SMILES: c1ccc(-c2ccc(Nc3nc(N4CCOCC4)c4oc5ncccc5c4n3)cc2)cc1] | 12 | ¹H NMR (DMSO-d₆) δ 9.49 (s, 1H), 8.62 (dd, 2H), 7.92 (d, 2H), 7.66 (m, 5H), 7.45 (dd, 2H), 7.28 (dd, 1H), 4.03 (m, 4H), 3.83 (m, 4H); LCMS (M + H): 424.2 m/z. |
| 45 | N-([1,1′- biphenyl]-3-yl)-4- morpholinopyrido [3′,2′:4,5]furo[3,2- d]pyrimidin-2- amine | 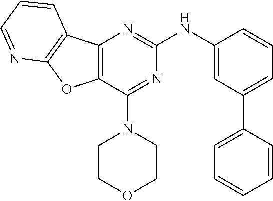 [SMILES: c1ccc(-c2cccc(Nc3nc(N4CCOCC4)c4oc5ncccc5c4n3)c2)cc1] | 12 | ¹H NMR (DMSO-d₆) δ 9.48 (s, 1H), 8.63 (d, 1H), 8.47 (m, 1H), 8.21 (s, 1H), 7.7-7.1 (m, 9H), 4.04 (m, 4H), 3.82 (m, 4H); LCMS (M + H): 424.1 m/z. |
| 46 | N-([1,1′- biphenyl]-2-yl)-4- morpholinopyrido [3′,2′:4,5]furo[3,2- d]pyrimidin-2- amine | 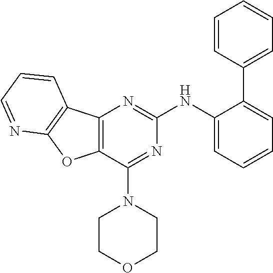 [SMILES: c1ccc(-c2ccccc2Nc2nc(N3CCOCC3)c3oc4ncccc4c3n2)cc1] | 12 | ¹H NMR (DMSO-d₆) δ 8.56 (d, 1H), 8.40 (d, 1H), 7.99 (br s, 1H), 7.90 (m, 1H), 7.55-7.35 (m, 6H), 7.29 (m, 2H), 7.19 (m, 1H), 3.86 (m, 4H), 3.73 (m, 4H); LCMS (M + H): 424.2 m/z. |
| 47 | N-(1,5-dimethyl- 1H-pyrazol-3-yl)- 4- morpholinopyrido [3′,2′:4,5]furo[3,2- d]pyrimidin-2- amine | 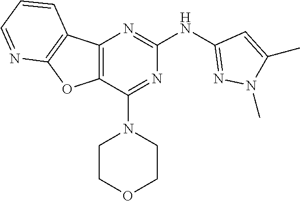 [SMILES: Cc1cc(Nc2nc(N3CCOCC3)c3oc4ncccc4c3n2)nn1C] | 12 | ¹H NMR (CDCl₃) δ 8.9 (d, 1H), 8.8 (d, 1H), 7.8 (m, 1H), 7.55 (br s, 1H), 6.9 (s, 1H), 4.45 (m, 4H), 4.25 (m, 4H), 4.05 (s, 3H), 2.65 (s, 3H); LCMS (M + H): 366.15 m/z. |
| 48 | 4-morpholino-N- (3-(o-tolyl)-1H- pyrazol-5- yl)pyrido[3′,2′:4,5] furo[3,2- d]pyrimidin-2- amine | 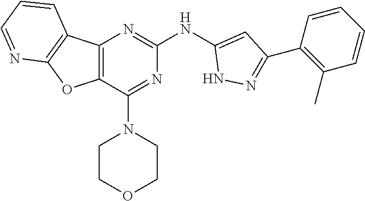 [SMILES: Cc1ccccc1-c1cc(Nc2nc(N3CCOCC3)c3oc4ncccc4c3n2)[nH]n1] | 7 | ¹H NMR (CDCl₃) δ 8.55 (m, 2H), 7.5 (m, 1H), 7.4 (d, 1H), 7.3 (s, 3H), 6.5 (s, 1H), 4.2 (m, 4H), 3.8 (m, 4H), 2.5 (s, 3H); LCMS (M + H): 428.1 m/z. |
| 49 | 4-morpholino-N- (3-(m-tolyl)-1H- pyrazol-5- yl)pyrido[3′,2′:4,5] furo[3,2- d]pyrimidin-2- amine | 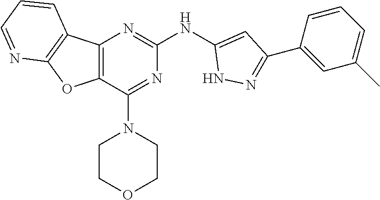 [SMILES: Cc1cccc(-c2cc(Nc3nc(N4CCOCC4)c4oc5ncccc5c4n3)[nH]n2)c1] | 7 | ¹H NMR (DMSO-d₆) δ 12.5 (s, 1H), 9.6 (d, 1H), 7.8-7.5 (m, 4H), 7.4 (m, 1H), 7.3 (m, 1H), 4.0 (m, 4H), 3.8 (m, 4H), 2.5 (s, 3H); LCMS (M + H): 428.2 m/z. |
| 50 | 4-morpholino-N- (3-(p-tolyl)-1H- pyrazol-5- yl)pyrido[3′,2′:4,5] furo[3,2- d]pyrimidin-2- amine | 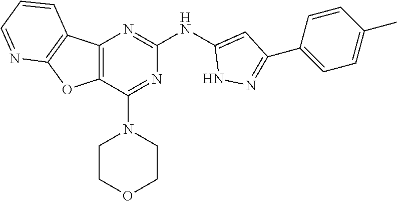 [SMILES: Cc1ccc(-c2cc(Nc3nc(N4CCOCC4)c4oc5ncccc5c4n3)[nH]n2)cc1] | 7 | ¹H NMR (acetone-d₆) δ 8.7 (br s, 1H), 8.61 (d, 1H), 7.74 ( m, 2H), 7.57 (t, 1H), 7.24 (d, 2H), 4.10 (m, 4H), 3.88 (m, 4H), 2.35 (s, 3H); LCMS (M + H): 428.2 m/z. |
| 51 | 8-bromo-4- morpholino-N-(5- phenyl-1H- pyrazol-3- yl)pyrido[3′,2′:4,5] furo[3,2- d]pyrimidin-2- amine | 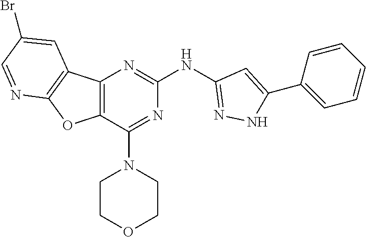 [SMILES: Brc1cnc2oc3c(N4CCOCC4)nc(Nc4cc(-c5ccccc5)[nH]n4)nc3c2c1] | 1 | ¹H NMR (CDl₃) δ 8.6 (s, 2H), 7.9 (m, 2H), 7.5-7.2 (m, 3H), 6.0 (s, 1H), 4.2 (m, 4H), 3.8 (m, 4H); LCMS (M + H): 492/494 m/z. |

*The compounds in this table were prepared generally according to the method identified in the table, substituting the appropriate starting materials.

### Example 52

### 4-Morpholinopyrido[3′,2′:4,5]furo[3,2-d]pyrimidine.

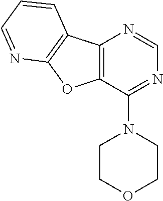 [SMILES: c1cnc2oc3c(N4CCOCC4)ncnc3c2c1]

A mixture of 2-chloro-4-morpholinopyrido[3′,2′:4,5]furo[3,2-d]pyrimidine (75 mg, 0.26 mmol), 10% Pd/C (82 mg), and ammonium formate (98 mg, 1.5 mmol) were stirred in methanol (1 mL) until complete conversion was observed. The reaction mixture was filtered through diatomaceous earth and the filtrate was concentrated. The crude material was subjected to reverse-phase chromatography (C18, SiO₂), eluting with 10-100% MeCN in water, to provide the titled compound (32 mg, 44%).

Where indicated, the compounds in the following table were prepared using methods analogous to those described Example Method 6 described above. The protected 3-aminopyrazoles used for Examples 53 to 63 were either purchased or prepared according to Steps A and B of Example Method 6 from the appropriate beta-ketonitriles. The beta-keto nitriles were prepared by reaction of acetonitrile anion with the appropriate ester according to the method found in: Nani, R. R., Reisman, S. E. (2013). α-Diazo-β-ketonitriles: Uniquely Reactive Substrates for Arene and Alkene Cyclopropanation. Journal of the American Chemical Society, 135(19), 7304-7311. The final compounds were obtained by reaction of 2-bromo-4-morpholinopyrido[3′,2′:4,5]furo[3,2-d]pyrimidine with the protected 3-aminpyrazole under the conditions outlined in Step C followed by deprotection according to Step D of Example Method 6.

TABLE 3

| Ex. | | | Ex | |
|---|---|---|---|---|
| # | Chemical Name | Structure | Method | LC-MS, NMR Data |
| 53 | N-(3-cyclopropyl-1H- pyrazol-5-yl)-4- morpholinopyrido[3′,2′:4,5] furo[3,2-d]pyrimidin- 2-amine hydrochloride | 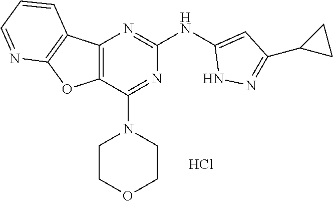 [SMILES: c1cnc2oc3c(N4CCOCC4)nc(Nc4cc(C5CC5)n[nH]4)nc3c2c1 ; Cl] | 6 | LC-MS (ESI+): m/z 378 (MH⁺) ¹H NMR (300 MHz, CD₃OD) δ 8.71- 8.66 (m, 2H), 7.65-7.61 (m, 1H), 5.83 (s, 1H), 4.27-4.22 (m, 4H), 3.91- 3.86 (m, 4H), 2.05-2.00 (m, 1H), 1.20-1.11 (m, 2H), 0.89-0.84 (m, 2H). |
| 54 | N-(3- (cyclopropylmethyl)- 1H-pyrazol-5-yl)-4- morpholinopyrido[3′,2′:4,5] furo[3,2-d]pyrimidin- 2-amine hydrochloride | 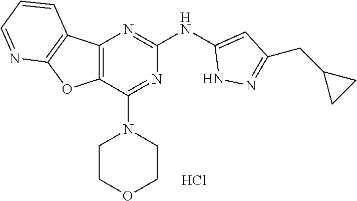 [SMILES: c1cnc2oc3c(N4CCOCC4)nc(Nc4cc(CC5CC5)n[nH]4)nc3c2c1 ; Cl] | 6 | LC-MS (ESI+): m/z 392 (MH⁺). ¹H NMR (300 MHz, CD₃OD) δ 8.74- 8.67 (m, 2H), 7.63 (dd, J = 7.8, 5.1Hz, 1H), 6.10 (s, 1H), 4.27-4.22 (m, 4H), 3.92-3.86 (m, 4H), 2.64 (d, J = 6.9 Hz, 2H), 1.09-1.03 (m, 1H), 0.65- 0.57 (m, 2H), 0.32-0.27 (m, 2H). |
| 55 | N-(3-cyclobutyl-1H- pyrazol-5-yl)-4- morpholinopyrido[3′,2′:4,5] furo[3,2-d]pyrimidin- 2-amine hydrochloride | 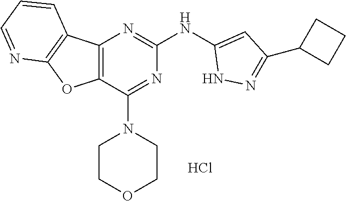 [SMILES: c1cnc2oc3c(N4CCOCC4)nc(Nc4cc(C5CCC5)n[nH]4)nc3c2c1 ; Cl] | 6 | LC-MS (ESI+): m/z 392 (MH⁺). ¹H NMR (300 MHz, CD₃OD) δ 8.70- 8.67 (m, 2H), 7.63 (dd, J = 7.8, 5.1Hz, 1H), 6.07 (s, 1H), 4.31-4.25 (m, 4H), 3.92-3.88 (m, 4H), 3.68-3.54 (m, 1H), 2.49- 2.41 (m, 2H), 2.34-2.12 (m, 3H), 2.09-1.97 (m, 1H). |
| 56 | N-(3- (cyclobutylmethyl)-1H- pyrazol-5-yl)-4- morpholinopyrido[3′,2′:4,5] furo[3,2-d]pyrimidin- 2-amine hydrochloride | 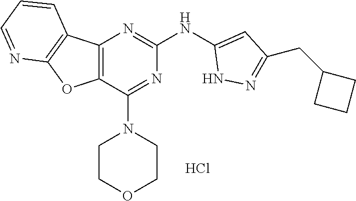 [SMILES: c1cnc2oc3c(N4CCOCC4)nc(Nc4cc(CC5CCC5)n[nH]4)nc3c2c1 ; Cl] | 6 | LC-MS (ESI+): m/z 406 (MH⁺) ¹H NMR (300 MHz, CD₃OD) δ 8.69- 8.67 (m, 2H), 7.63 (dd, J = 7.8, 5.1Hz, 1H), 5.98 (s, 1H), 4.28-4.22 (m, 4H), 3.93-3.88 (m, 4H), 2.92-2.80 (m, 2H), 2.73- 2.63 (m, 1H), 2.16-2.12 (m, 2H), 1.99-1.76 (m, 4H). |
| 57 | 4-morpholino-N-(3- (trifluoromethyl)-1H- pyrazol-5- yl)pyrido[3′,2′:4,5]furo [3,2-d]pyrimidin-2-amine hydrochloride | 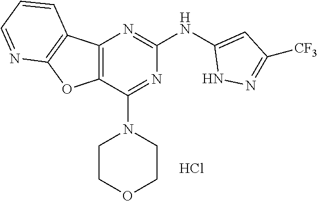 [SMILES: FC(F)(F)c1cc(Nc2nc(N3CCOCC3)c3oc4ncccc4c3n2)[nH]n1 ; Cl] | 6 | LC-MS (ESI+): m/z 406 (MH⁺). ¹H NMR (300 MHz, DMSO-d₆) δ 10.18 (s, 1H), 8.94 (d, J = 7.5 Hz, 1H), 8.62 (d, J = 3.6 Hz, 1H), 7.58 (dd, J = 7.8, 5.4 Hz, 1H), 6.24 (s, 1H), 4.05-4.01 (m, 4H), 3.86-3.80 (m, 4H). |
| 58 | N-(3-benzyl-1H-pyrazol- 5-yl)-4- morpholinopyrido[3′,2′:4,5] furo[3,2-d]pyrimidin- 2-amine hydrochloride | 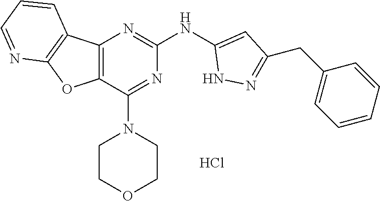 [SMILES: c1ccc(Cc2cc(Nc3nc(N4CCOCC4)c4oc5ncccc5c4n3)[nH]n2)cc1 ; Cl] | 6 | LC-MS (ESI+): m/z 428 (MH⁺) ¹H NMR (300 MHz, CD₃OD) δ 8.67- 8.64 (m, 2H), 7.62 (dd, J = 7.5, 5.1Hz, 1H), 7.34- 7.24 (m, 5H), 5.96 (s, 1H), 4.26-4.19 (m, 4H), 4.07 (s, 2H), 3.92-3.86 (m, 4H). |
| 59 | N-(3-cyclopentyl-1H- pyrazol-5-yl)-4- morpholinopyrido[3′,2′:4,5] furo[3,2-d]pyrimidin- 2-amine hydrochloride | 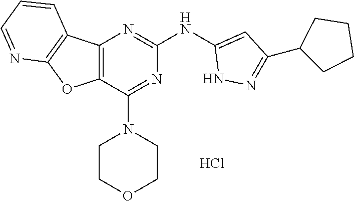 [SMILES: c1cnc2oc3c(N4CCOCC4)nc(Nc4cc(C5CCCC5)n[nH]4)nc3c2c1 ; Cl] | 6 | LC-MS (ESI+): m/z 406 (MH⁺). ¹H NMR (300 MHz, DMSO-d₆) δ 11.80 (s, 1H), 9.10 (s, 1H), 8.60-8. 48 (m, 2H), 7.56-7.52 (m, 1H), 6.45 (s, 1H), 4.08-3.95 (m, 4H), 3.90-3.79 (m, 4H), 3.10-2.98 (m, 1H), 2.10- 1.90 (m, 2H), 1.72-1.62 (m, 6H). |
| 60 | 4-morpholino-N-(3- (thiophen-2-yl)-1H- pyrazol-5- yl)pyrido[3′,2′:4,5]furo [3,2-d]pyrimidin-2-amine hydrochloride | 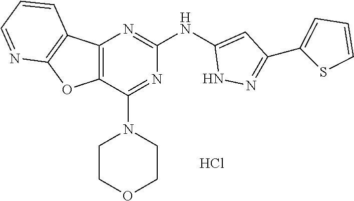 [SMILES: c1csc(-c2cc(Nc3nc(N4CCOCC4)c4oc5ncccc5c4n3)[nH]n2)c1 ; Cl] | 6 | LC-MS (ESI+): m/z 420 (MH⁺). ¹H NMR (300 MHz, CD₃OD) δ 8.72 (dd, J = 4.8, 1.5 Hz, 1H), 8.65 (dd, J = 5.1, 1.8 Hz, 1H), 7.67 (dd, J = 7.8, 4.8 Hz, 1H), 7.55 (d, J = 5.1 Hz, 1H), 7.49 (d, J = 3.0 Hz, 1H), 7.17 (dd, J = 5.1, 3.9 Hz, 1H), 6.34 (s, 1H), 4.40-4.31 (m, 4H), 3.96-3.91 (m, 4H). |
| 61 | 4-morpholino-N-(3- (thiophen-3-yl)-1H- pyrazol-5- yl)pyrido[3′,2′:4,5]furo [3,2-d]pyrimidin-2-amine hydrochloride | 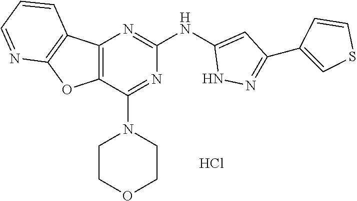 [SMILES: c1cnc2oc3c(N4CCOCC4)nc(Nc4cc(-c5ccsc5)n[nH]4)nc3c2c1 ; Cl] | 6 | LC-MS (ESI+): m/z 420 (MH⁺). ¹H NMR (300 MHz, DMSO-d₆) δ 8.75 (d, J = 7.8 Hz, 1H), 8.68-8.66 (m, 1H), 7.96 (s, 1H), 7.68-7.57 (m, 3H), 6.68 (s, 1H), 4.16- 4.03 (m, 4H), 3.90-3.81 (m, 4H). |
| 62 | N-(3-ethyl-1H-pyrazol- 5-yl)-4- morpholinopyrido[3′,2′:4,5] furo[3,2-d]pyrimidin- 2-amine hydrochloride | 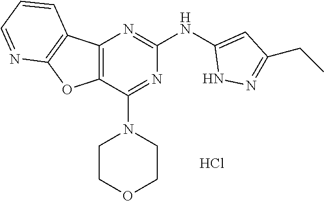 [SMILES: CCc1cc(Nc2nc(N3CCOCC3)c3oc4ncccc4c3n2)[nH]n1 ; Cl] | 6 | LC-MS (ESI+): m/z 366 (MH⁺). ¹H NMR (300 MHz, CD₃OD) δ 8.80- 8.60 (m, 2H), 7.62-7.60 (m, 1H), 6.00 (s, 1H), 4.26-4.21 (m, 4H), 3.90- 3.81 (m, 4H), 2.77 (q, J = 7.5 Hz, 2H), 1.33 (t, J = 7.5 Hz, 3H). |
| 63 | N-(3-isopropyl-1H- pyrazol-5-yl)-4- morpholinopyrido[3′,2′:4,5] furo[3,2-d]pyrimidin- 2-amine hydrochloride | 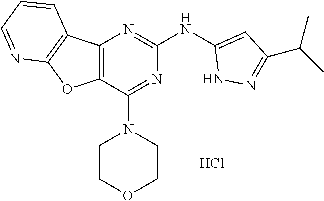 [SMILES: CC(C)c1cc(Nc2nc(N3CCOCC3)c3oc4ncccc4c3n2)[nH]n1 ; Cl] | 6 | LC-MS (ESI+): m/z 380 (MH⁺). ¹H NMR (300 MHz, CD₃OD) δ 8.80- 8.60 (m, 2H), 7.62-7.60 (m, 1H), 6.00 (s, 1H), 4.26-4.21 (m, 4H), 3.92- 3.85 (m, 4H), 3.11-3.06 (m, 1H), 1.35 (d, J = 6.9 Hz, 6H). |
| 64 | N-(3- (cyclopentylmethyl)-1H- pyrazol-5-yl)-4- morpholinopyrido[3′,2′:4,5] furo[3,2-d]pyrimidin- 2-amin | 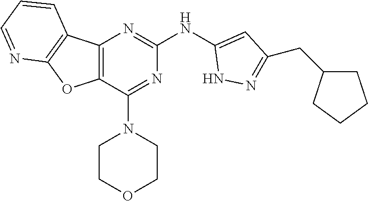 [SMILES: c1cnc2oc3c(N4CCOCC4)nc(Nc4cc(CC5CCCC5)n[nH]4)nc3c2c1] | | |

Biological Example 1: Inhibition of PIKfyve

Full length human recombinant PIKFYVE expressed in baculovirus expression system as N-terminal GST-fusion protein (265 kDa) was obtained from Carna Biosciences (Kobe, Japan). The kinase substrate was prepared by mixing and sonicating fluorescently-labeled phosphatidylinositol 3-phosphate (PI3P) with phospho-L-serine (PS) at a 1:10 ratio in 50 mM HEPES buffer pH_{7.5.}

The kinase reactions were assembled in 384-well plates (Greiner) in a total volume of 20 mL as follows. Kinase protein was pre-diluted in an assay buffer comprising 25 mM HEPES, pH 7.5, 1 mM DTT, 2.5 mM MgCl₂, and 2.5 mM MnCl₂, and 0.005% Triton X-100, and dispensed into a 384-well plate (10 μL per well). Test compounds were serially pre-diluted in DMSO and added to the protein samples by acoustic dispensing (Labcyte Echo). The concentration of DMSO was equalized to 1% in all samples. All test compounds were tested at 12 concentrations. Apilimod was used as a reference compound and was tested in identical manner in each assay plate. Control samples (0%-inhibition, in the absence of inhibitor, DMSO only) and 100%-inhibition (in the absence of enzyme) were assembled in replicates of four and were used to calculate %-inhibition in the presence of compounds. The reactions were initiated by addition of 10 μL of 2× PI3P/PS substrate supplemented with ATP. The final concentration of enzyme was 2 nM, the final concentration of ATP was 10 mM, and the final concentration of PI3P/PS substrate was 1 μM (PI3P). The kinase reactions were allowed to proceed for 3 h at room temperature. Following incubation, the reactions were quenched by addition of 50 mL of termination buffer (100 mM HEPES, pH 7.5, 0.01% Triton X-100, 20 mM EDTA). Terminated plates were analyzed on a microfluidic electrophoresis instrument (Caliper LabChip® 3000, Caliper Life Sciences/Perkin Elmer). The change in the relative fluorescence intensity of the PI(3)P substrate and PI(3,5)P product peaks was measured. The activity in each test sample was determined as the product to sum ratio (PSR): P/(S+P), where P is the peak height of the product, and S is the peak height of the substrate. Percent inhibition (Pᵢₙₕ) was determined using the following equation:

Pᵢₙₕ=(PSR_{0%inh}−PSR_{compound})/(PSR_{0%inh}−P_{100%inh})*100

in which PSR_{compound} is the product/sum ratio in the presence of compound, PSR_{0%inh} is the product/sum ratio in the absence of compound, and the PSR_{100%inh} is the product/sum ratio in the absence of the enzyme. To determine the ICso of test compounds (50%-inhibition) the %-inh cdata (Pᵢₙₕ versus compound concentration) were fitted by a four-parameter sigmoid dose-response model using XLfit software (IDBS).

The IC₅₀ values for certain compounds of the disclosure are provided in Table 4 below.

TABLE 4

| | PIKfyve |
|---|---|
| Ex. | IC₅₀ (nM) |
| 1 | 473 |
| 2 | >10,000 |
| 3 | >10,000 |
| 4 | 68 |
| 5 | 17 |
| 6 | 251 |
| 7 | 573 |
| 8 | 2200 |
| 9 | 23 |
| 10 | 185 |
| 11 | >10000 |
| 12 | >10000 |
| 13 | 143 |
| 14 | 2320 |
| 15 | 108 |
| 16 | 129 |
| 17 | 129 |
| 18 | 1630 |
| 19 | 2180 |
| 20 | 4750 |
| 21 | 988 |
| 22 | 6400 |
| 23 | 20 |
| 24 | 61 |
| 25 | 14 |
| 26 | 82 |
| 27 | 16 |
| 28 | 46 |
| 29 | 3 |
| 30 | 163 |
| 31 | 1090 |
| 32 | 825 |
| 33 | 261 |
| 34 | 303 |
| 35 | 1540 |
| 36 | >10000 |
| 37 | 439 |
| 38 | >10000 |
| 39 | 2180 |
| 40 | 2770 |
| 41 | >10000 |
| 42 | >10000 |
| 43 | >10000 |
| 44 | 356 |
| 45 | 3980 |
| 46 | 3870 |
| 47 | 4320 |
| 48 | 515 |
| 49 | 38 |
| 50 | 398 |
| 51 | 862 |
| 52 | >10000 |
| 53 | 2090 |
| 54 | 2600 |
| 55 | 1280 |
| 56 | 1790 |
| 57 | 5900 |
| 58 | 831 |
| 59 | 1290 |
| 60 | 37 |
| 61 | 26 |
| 62 | 7850 |
| 63 | 1850 |

Biological Example 2: Inhibition of PI3K Isoforms

The enzyme preparations shown in Table 5 were used.

TABLE 5

| Assay name | Catalytic subunit | Regulatory subunit | Expression host |
|---|---|---|---|
| PI3Kα | Full-length N-terminal | Full length, human | SF9 cells |
| (p110α/p85α) | FLAG-tagged human p110α | (no tag) p85α | (baculovirus) |
| PI3Kβ | Full-length N-terminal GST- | Full length, human | SF9 cells |
| (p110β/p85α) | tagged human p110β | (no tag) p85α | (baculovirus) |
| PI3Kδ | Full-length N-terminal GST- | Full length, human | SF9 cells |
| (p110δ/p85α) | tagged human p110δ | (no tag) p85α | (baculovirus |
| PI3Kγ (p120γ) | Full length N-terminal His- | None | SF9 cells |
| | tagged human p120γ | | (baculovirus) |

The kinase substrate was prepared by mixing and sonicating fluorescently-labeled phosphatidylinositol 4,5-phosphate (PIP2) with phospho-L-serine (PS) at 1:20 ratio in 50 mM HEPES buffer pH_{7.5.}

The kinase reactions were assembled in 384-well plates (Greiner) in a total volume of 20 mL as follows. The kinase proteins were pre-diluted in an assay buffer comprising 50 mM HEPES, pH 7.5, 0.012% CHAPS, 1 mM DTT, 10 mM Na₃VaO₄, 10 mM β-GP, 3 mM MgCl₂, and 40 mM NaCl₂, and dispensed into a 384-well plate (10 μL per well). Test compounds were serially pre-diluted in DMSO and added to the protein samples by acoustic dispensing (Labcyte Echo). The concentration of DMSO was equalized to 1% in all samples. All test compounds were tested at 12 concentrations. The control samples (0%-inhibition in the absence of inhibitor, DMSO only) and 100%-inhibition (in the absence of enzyme) were assembled in replicates of four and were used to calculate %-inhibition in the presence of test compounds. The reactions were initiated by addition of 10 μL of the PIP2/PS substrate supplemented with ATP. The final concentration of enzymes was 0.5 nM (PI3Kα), 1 nM (PI3Kβ), 10 nM (PI3Kγ), and 0.25 nM (PI3Kδ). The final concentration of ATP was 90 μM (PI3Ka), 60 μM (PI3Kβ), 100 μM (PI3Kγ), and 90 μM (PI3Kδ). The final concentration of PIP2/PS substrate was 1μM (PIP2). The kinase reactions were allowed to proceed for 3 h at room temperature. Following incubation, the reactions were quenched by addition of 50 μL of termination buffer (100 mM HEPES, pH 7.5, 0.01% Triton X-100, 20 mM EDTA). Terminated plates were analyzed on a microfluidic electrophoresis instrument (Caliper LabChip® 3000, Caliper Life Sciences/Perkin Elmer). The change in the relative fluorescence intensity of the PI(4,5)P substrate and PI(3,4,5)P product peaks was measured. The activity in each test sample was determined as the product to sum ratio (PSR): P/(S+P), where P is the peak height of the product, and S is the peak height of the substrate. Percent inhibition (Pᵢₙₕ) was determined using the following equation:

Pᵢₙₕ=(PSR_{0%inh}−PSR_{compound})/(PSR_{0%inh}−P_{100%inh})*100

in which PSR_{compound} is the product/sum ratio in the presence of compound, PSR_{0%inh} is the product/sum ratio in the absence of compound, and the PSR_{100%inh} is the product/sum ratio in the absence of the enzyme. To determine the ICso of test compounds (50%-inhibition), the %-inh cdata (Pion versus compound concentration) were fitted by a four-parameter sigmoid dose-response model using XLfit software (IDBS).

Biological Example 3: HEK/TDP Survival assay

Immortalized human embryonic kidney 293T (HEK 293T) were transfected with plasmids containing TDP-43 Q331K mutation, resulting in an increase in cell death that is biologically relevant to ALS patients. Cell death is measured as reductions in the amount of ATP, an indicator of metabolically active cells, that is quantified by a luminescence Cell-Titer-Glo® (CTG) reagent. Compounds are evaluated in this model for changes in CTG compared to no treatment group. Increased signal indicates improved survival (rescue) and decreased signal indicates decreased survival. Cell rescue was measured in a 96-well format with eight different concentrations of the test compound over 48hrs with 6 replicates. The Promega Cell-Titer-Glo® Luminescent Cell Viability Assay was used to quantify ATP, an indicator of metabolically active cells (see protocol: https://www.promega.com/-/media/files/resources/protocols/technical-manuals/101/celltiterglo-2-0-assay-protocol.pdf?la=en). The luminescence signal was detected using the PerkinElmer EnVision or Molecular Devices SpectraMax.

The effect of a compound at a given dose on cell viability was determined using a three step procedure. First, we computed Hedge's g for the Cell Titer-Glo luminescence values using six untreated wells on every plate as a control. Second, as multiple experimental trials of each compound-dose pair were performed, these results were meta-analyzed to produce a single estimate of the effect size. Finally, values from all compound-dose pairs were corrected for multiple hypothesis testing using the Empirical Bayes framework of Stephens, M. (False discovery rates: a new deal, Biostatistics, 18 [2], 2017, 275-294) yielding credible intervals for the measured effect and associated s values. Briefly, this method computes a local false sign rate for each experiment. Analogous to the local false discovery rate of Efron, B. (Size, power and false discovery rates, Ann. Statist. 35 [4], 2007, 1351-1377) this value measures the confidence in the sign of each effect (rather than confidence in each effect being non-zero). The s values reported in the previous figures are the expected fraction of errors if we were to estimate the sign of all effects with greater absolute local false sign rate, defined in analogy to the q value of Storey, J D (The positive false discovery rate: a Bayesian interpretation and the q-value, Ann. Statist. 31 [6] 2003, 2013-2035). Compounds that yielded signed logs values greater than 3 were considered hits. This threshold was determined by a separate calibration experiment in which Cell Titer-Glo® was measured in blank plates consisting of untreated cells to assess the noise inherent in the assay. Data are presented as the maximal effect of rescue obtained from the dose-response curve.

Biological Example 4: iPSC MN Survival assay

Fibroblasts from ALS patients with known SOD1 A4V mutation were reprogrammed into inducible pluripotent stem cells (iPSC) and then differentiated to motor neurons. In culture, ALS patient derived motor neurons show increased death rate compared to motor neurons derived from healthy individuals in a stressed condition (nutrient deprived media, Hank's buffered salt solution—HBSS). The SOD1 survival deficit is relevant to a subset of ALS patient biology and serves as a suitable cell-based model for gauging compound induced survival rescue. Cell rescue was measured following more than two different concentrations of each compound for six days with greater than four replicates in a 96-well format to ensure studies with power >0.8. Cells are transduced with a GFP reporter and imaged once a day to track survival. A broad-spectrum caspase inhibitor served as the positive control.

Microscopy image-based readout: Cells are transduced with a GFP reporter and imaged once a day with a blue laser to track survival. Imagers used include the Biotek Cytation 5 and Thermo Fisher EVOS Auto FL 2. All Images undergo uniform processing consisting of rolling hat background subtraction and contrast adjustment. Microscopy images are analyzed using software proprietary to Verge. Cells are identified by their shape and each cell is tracked across images and time points for each well. Survival is visually assessed from the Kaplan-Meier curves. Survival of the cells is modeled and tested using a mixed effects Cox regression where each well is modeled as the random effect, and the group (control/treatment) as the fixed effect. Hazard ratios between treatment and control are estimated within the Cox regression where a value of 1.0 denotes no change, values >1.0 indicate decreased survival in response to treatment, and values <1.0 indicate increased survival in response to treatment. Data are presented in Table 7 as the maximal reduction in hazard ratio scores measured at various concentrations.

The ICso values for certain compounds of the disclosure are provided in Table 6 below.

TABLE 6

| | PI3Kα | | PI3Kδ | |
|---|---|---|---|---|
| Ex. | IC₅₀ (nM) | PI3Kβ IC₅₀ (nM) | IC₅₀ (nM) | PI3Kγ IC₅₀ (nM) |
| 8 | 161 | 306 | 23.9 | 2610 |
| 9 | 144 | >10000 | 75 | >10000 |
| 10 | 117 | 1310 | 37 | 582 |
| 11 | 244 | 7030 | 278 | 4668 |
| 12 | 1640 | 6540 | 1890 | >10000 |
| 21 | 158 | 243 | 37.4 | 1410 |
| 22 | 802 | 2700 | 464 | >10000 |
| 36 | 77 | 749 | 77 | 2650 |
| 37 | 67 | 1040 | 41 | 7280 |
| 38 | 8740 | 5820 | 1490 | >10000 |
| 39 | 82 | 370 | 60 | 677 |
| 40 | 102 | 1190 | 66 | 2040 |
| 47 | 82 | 2630 | 54 | 1870 |
| 55 | 16 | 315 | 7 | 206 |
| 56 | 47 | 937 | 24 | 1540 |

Cell survival data are provided in Table 7 below.

TABLE 7

| | IPSC-C9 | IPSC-SOD1 | HEK/TDP (uM, |
|---|---|---|---|
| Ex. | (Hazard Ratio) | (Hazard Ratio) | Maximum Rescue dose) |
| 9 | 0.65 | 0.81 | 0.2 |
| 10 | 0.96 | 1.04 | 0 |
| 11 | 0.99 | 1.1 | 0 |
| 14 | 0.9 | 0.96 | 0.19 |
| 37 | 0.99 | 1.03 | 0.1 |
| 43 | 1.06 | 1.1 | 0.34 |
| 47 | | 1.1 | 0 |
| 49 | 1.2 | 1.03 | 0.13 |
| 61 | 0.542 | | |

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed is:

1. A compound of Formula (I): 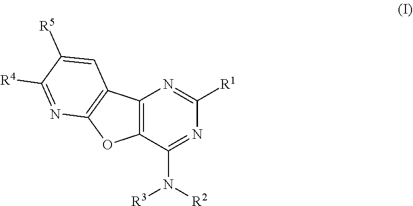
wherein:
R¹ is H, C₁₋₄alkyl, or —NR^{a}R^{b};
wherein R^{a} is H or C₁₋₄alkyl; and
R^{b} is —L—R^{c};
wherein L is a bond, —C(O)—, —C(O)O—, or —C₁₋₄alkylene-; and
R^{c} is phenyl, monocyclic cycloalkyl, monocyclic heterocycloalkyl, or monocyclic heteroaryl, wherein each phenyl, cycloalkyl, heterocycloalkyl, or heteroaryl is optionally substituted with one, two, or three R^{d} substituents;
wherein each R^{d} substituent is independently C₁₋₄alkyl, C₁₋₄alkenyl, C₁₋₄alkynyl, —O—C₁₋₄alkyl, halo, cyano, nitro, azido, halo—C₁₋₄alkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h},=NOR_{g}, —NR^{g}S(═O)₁₋₂R^{h}, —NR^{g}S(═O)₁₋₂NR^{g}R^{h}, ═NSO2R_{g}, —C(═O)R_{g}, —C(═O)OR_{g}, —OC(═O)OR_{g}, —OC(═O)R_{g}, —C(═O)NR^{g}R^{h}, —OC(═O)NR^{g}R^{h}, —OR_{g}, —SR_{g}, —S(═O)R_{g}, —S(═O)₂R_{g}, —OS(═O)₁₋₂R_{g}, —S(═O)₁-20R_{g}, —S(═O)₁₋₂NR^{g}R^{h}, phenyl, —C₁₋₄alkyl-phenyl, monocyclic cycloalkyl, —C₁₋₄alkyl-cycloalkyl, monocyclic heterocycloalkyl, or monocyclic heteroaryl;
wherein each phenyl, monocyclic cycloalkyl, monocyclic heterocycloalkyl, or monocyclic heteroaryl of R^{d} is optionally substituted with one, two, or three substituents R^{e};
wherein each R^{e} substituent is independently C₁₋₄alkyl, C₁₋₄alkenyl, C₁₋₄alkynyl, halo, cyano, nitro, azido, —OH, halo—C₁₋₄alkyl, —O—C₁₋₄alkyl, or —O—C₁₋₄-haloalkyl;
R^{g} and R^{h} are each independently H or C₁₋₄alkyl;
R² and R³ taken together with the nitrogen to which they are attached form a monocyclic heterocycloalkyl, optionally substituted with one, two, or three R¹ substituents;
wherein each R¹ substituent is independently C₁₋₄alkyl, —OH, —NR^{k}R^{l}, halo, halo—C₁₋₄alkyl, —O—C₁₋₄alkyl, or —O—C₁₋₄-haloalkyl;
where R^{k} and R^{I} are each independently H or C₁₋₄alkyl; and
R⁴ and R⁵ are each independently H, C₁₋₄alkyl, halo, —OH, or —OC₁₋₄alkyl, wherein each alkyl is optionally substituted with —NR^{m}Rⁿ;
wherein R^{m} and Rⁿ are each independently H or C₁₋₄alkyl, or R^{m} and Rⁿ taken together with the nitrogen to which they are attached form a monocyclic heterocycloalkyl, optionally substituted with one or two R^{o} substituents;
wherein each R^{o} substituent independently C₁₋₄alkyl, —OH, —OC₁₋₄alkyl, halo, cyano, or —NR^{p}R^{q};
wherein R^{p} and R^{q} are each independently H or C₁₋₄alkyl;
or a pharmaceutically acceptable salt thereof.

2. The compound of claim 1, wherein R¹ is —NR^{a}R^{b}.

3. The compound of claim 1 or claim 2, wherein R^{a} is H.

4. The compound of claim 1 or claim 2, wherein R^{a} is C₁₋₄alkyl.

5. The compound of any one of the preceding claims, wherein L is a bond.

6. The compound of any one of claims 1 to 4, wherein L is —C(O)— or —C(O)O—.

7. The compound of any one of claims 1 to 4, wherein L is —C₁₋₄alkylene-.

8. The compound of claim 7, wherein L is methylene or ethylene.

9. The compound of any one of claims 1 to 8, wherein R^{c} is optionally substituted phenyl.

10. The compound of any one of claims 1 to 8, wherein R^{c} is optionally substituted monocyclic cycloalkyl.

11. The compound of claim 10, wherein R^{c} is optionally substituted cyclopropyl, cyclobutyl, cyclopentyl, or cyclohexyl.

12. The compound of claim 11, wherein R^{c} is optionally substituted cyclopropyl.

13. The compound of any one of claims 1 to 8, wherein R^{c} is optionally substituted monocyclic heterocycloalkyl.

14. The compound of claim 13, wherein R^{c} is optionally substituted pyrrolidinyl, tetrahydrofuranyl, piperidinyl, morpholinyl, or piperazinyl.

15. The compound of any one of claims 1 to 8, wherein R^{c} is optionally substituted monocyclic heteroaryl.

16. The compound of claim 15, wherein R^{c} is optionally substituted pyrrole, imidazole, pyrazole, triazole, tetrazole, furan, oxazole, isoxazole, thiazole, isothiazole, pyridine, pyrimidine, pyrazine, or pyridazine.

17. The compound of claim 16, wherein R^{c} is optionally substituted pyrazole, imidazole, pyridine, or pyrimidine.

18. The compound of claim 16, wherein R^{c} is optionally substituted pyrazole.

19. The compound of any one of claims 1 to 18, wherein each R^{e} is optionally substituted with one or two R^{d} substituents.

20. The compound of any one of claims 1 to 19, wherein each R^{d} substituent is independently C₁₋₄alkyl, C₁₋₄alkenyl, C₁₋₄alkynyl, —O—C₁₋₄alkyl, halo, cyano, nitro, azido, halo-C₁₋₄alkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR_{g}, —NR^{g}S(═O)₁₋₂R^{h}, —NR^{g}S(═O)₁₋₂NR^{g}R^{h},=NSO2R_{g}, —C(═O)R_{g}, —C(═O)OR_{g}, —OC(═O)OR_{g}, —OC(═O)R_{g}, —C(═O)NR^{g}R^{h}, —OC(═O)NR^{g}R^{h}, —OR_{g}, —SR_{g}, —S(═O)R_{g}, —S(═O)₂R_{g}, —OS(═O)₁₋₂R_{g}, —S(═O)₁-20R_{g}, —S(═O)₁₋₂NR^{g}R^{h,} ₚhenyl, —C₁₋₄alkyl-phenyl, monocyclic cycloalkyl, —C₁₋₄alkyl-cycloalkyl, monocyclic heterocycloalkyl, or monocyclic heteroaryl, wherein the phenyl, monocyclic cycloalkyl, monocyclic heterocycloalkyl, and monocyclic heteroaryl of R^{d} are each optionally substituted with one or two substituents

21. The compound of claim 20, wherein each R^{d} substituent is independently C₁₋₄alkyl, halo-C₁₋₄alkyl, phenyl, —C₁₋₄alkyl-phenyl, pyridyl, thiophenyl, cycloalkyl, or —C₁₋₄alkyl-cycloalkyl, wherein the phenyl, pyridyl, and thiophenyl are each optionally substituted with one or two substituents R^{e}.

22. The compound of claim 21, wherein each R^{d} substituent is independently methyl, ethyl isopropyl, —CF₃, —OCH₃, —OCF₃, phenyl, pyridyl, thiophenyl, benzyl, cyclopropyl, cyclobutyl, cyclopentyl, cyclopropylmethyl, cyclobutylmethyl, or cyclopentylmethyl, wherein the phenyl, cycloalkyl, and heteroaryl of R^{d} are each optionally substituted with one or two substituents R^{e}.

23. The compound of claim 22, wherein each R^{d} substituent is independently methyl, ethyl isopropyl, —CF₃, phenyl, pyridyl, thiophenyl, benzyl, cyclopropyl, cyclobutyl, cyclopentyl, cyclopropylmethyl, cyclobutylmethyl, or cyclopentylmethyl, wherein each phenyl, benzyl, cyclopropyl, cyclobutyl, cyclopentyl, cyclopropylmethyl, cyclobutylmethyl, or cyclopentylmethyl is optionally substituted with one or two substituents R^{e}, wherein each R^{e} is independently methyl, —CF₃, fluoro, chloro, —OCH₃, or —OCF₃.

24. The compound of any one of claims 1 to 22, wherein each R^{e} substituent is independently C₁₋₄alkyl, halo, halo—C₁₋₄alkyl, —O—C₁₋₄alkyl, or —O—C₁₋₄-haloalkyl.

25. The compound of claim 24, wherein each R^{e} substituent is independently methyl, —CF₃, fluoro, chloro, —OCH₃, or —OCF₃.

26. The compound of any one of claims 1 to 20, wherein R^{g} and R^{h} are each independently H or methyl.

27. The compound of claim 1, wherein R¹ is H.

28. The compound of claim 1, wherein R¹ is C₁₋₄alkyl.

29. The compound of any one of the preceding claims, wherein R² and R³ taken together with the nitrogen to which they are attached form pyrrolidine, piperidine, piperazine, morpholine, thiomorpholine, or thiomorpholine-1,1-dioxide, each optionally substituted with one, two, or three R¹ substituents.

30. The compound of claim 29, wherein R² and R³ taken together with the nitrogen to which they are attached form morpholine, optionally substituted with one or two R^{j} substituents.

31. The compound of any one of the preceding claims, wherein each substituent is independently methyl, hydroxy, —OCH₃, halo, —CF₃, or —OCF₃.

32. The compound of any one of claims 1 to 30, wherein R^{k} and R^{I} are each independently H or methyl.

33. The compound of any one of the preceding claims, wherein R⁴ and R⁵ are each H.

34. The compound of any one of claims 1 to 32, wherein one of R⁴ and R⁵ is H and the other is C₁₋₄alkyl, halo, —OH, or —OC₁₋₄alkyl, wherein each alkyl is optionally substituted with —NR^{m}Rⁿ.

35. The compound of claim 34, wherein one of R⁴ and R⁵ is H and the other is —OH, halo, or —OCH₃.

36. The compound of claim 34, wherein one of R⁴ and R⁵ is H and the other is C₂₋₃alkyl substituted with —NR^{m}Rⁿ.

37. The compound of claim 34 or claim 36, wherein R^{m} and Rⁿ are each independently H or C₁₋₄alkyl.

38. The compound of claim 37, wherein R^{m} and Rⁿ are each methyl.

39. The compound of claim 34 or claim 36, wherein R^{m} and Rⁿ taken together with the nitrogen to which they are attached form a monocyclic heterocycloalkyl, optionally substituted with one or two R^{o} substituents.

40. The compound of claim 39, wherein R^{m} and Rⁿ taken together with the nitrogen to which they are attached form pyrrolidine, piperidine, piperazine, morpholine, thiomorpholine, or thiomorpholine-1,1-dioxide, each optionally substituted with one or two R^{o} substituents.

41. The compound of claim 39, wherein R^{m} and Rⁿ taken together with the nitrogen to which they are attached form pyrrolidine, piperidine, piperazine, or morpholine, each optionally substituted with one or two R^{o} substituents.

42. The compound of any one of claims 39 to 41, wherein each R^{o} substituent is C₁₋₄alkyl.

43. The compound of claim any one of claims 39 to 41, wherein R^{p} and R^{q} are each independently H or methyl.

44. A compound of Formula (II): 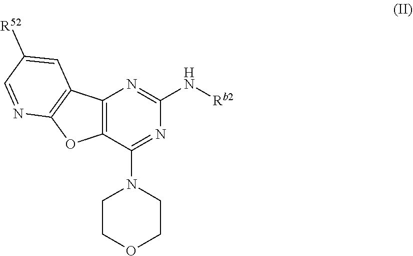
wherein
R^{b2} is pyrazole, phenyl, pyridyl, or pyrimidinyl, each optionally substituted with one or two substituents selected from C₁₋₄alkyl, phenyl, and pyridyl,
wherein the phenyl and pyridyl substituents are optionally substituted with methyl, —CF₃, fluoro, chloro, —OCH₃, —OCF₃, or phenyl;
R⁵² is H or —C₂₋₃-alkylene—NR^{m2}Rⁿ²;
wherein R^{m2} and Rⁿ² are each independently H or methyl, or R^{m2} and Rⁿ² taken together with the nitrogen to which they are attached form pyrrolidine, piperidine, piperazine, or morpholine, each optionally substituted with methyl;
or a pharmaceutically acceptable salt thereof.

45. The compound of claim 44, wherein R^{b2} is pyrazole, optionally substituted with methyl, —CF₃, fluoro, chloro, —OCH₃, —OCF₃, or phenyl.

46. A compound of Formula (III): 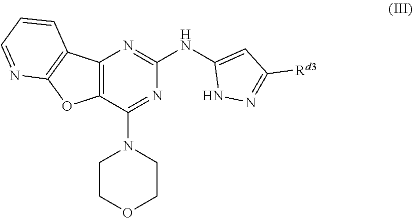
wherein
R^{d3} is (a) methyl, ethyl, isopropyl, —CF₃, —OCH₃, or —OCF₃, or (b) phenyl, benzyl, cyclopropyl, cyclobutyl, cyclopentyl, cyclopropylmethyl, cyclobutylmethyl, cyclopentylmethyl, thiophenyl, or pyridyl, wherein the phenyl, benzyl, each optionally substituted with one or two substituents independently selected from methyl, fluoro, chloro, —CF₃, —OCH₃, and —OCF₃;
or a pharmaceutically acceptable salt thereof.

47. A compound selected from any of the compounds in Table 1, and pharmaceutically acceptable salts thereof.

48. A pharmaceutical composition comprising a compound and/or a pharmaceutically acceptable salt of any one of claims 1 to 47 and a pharmaceutically acceptable excipient.

49. A method of inhibiting PIKfyve and/or a PI3 kinase in a subject in need thereof comprising administering to the subject an effective amount of a compound of any one of claims 1 to 47, or a pharmaceutical composition of claim 48.

50. A method of treating a neurological disease associated with PIKfyve activity and/or PI3 kinase activity in a subject in need thereof comprising administering to the subject an effective amount of a compound of any one of claims 1 to 47, or a pharmaceutical composition of claim 48.

51. The method of claim 50, wherein the disease is associated with PIKfyve activity.

52. The method of claim 50, wherein the disease is amyotrophic lateral sclerosis (ALS), primary lateral sclerosis (PLS), Charcot-Marie-Tooth (CMT; including type 4J (CMT4J)), and Yunis-Varon syndrome, autophagy, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, fronto-temporal dementia, diseases of neuronal nuclear inclusions of polyglutamine and intranuclear inclusion bodies, disease of Marinesco and Hirano bodies, tauopathy, Alzheimer's disease, neurodegeneration, spongiform neurodegeneration, peripheral neuropathy, leukoencephalopathy, inclusion body disease, progressive supranuclear palsy, corticobasal syndrome, chronic traumatic encephalopathy, traumatic brain injury (TBI), cerebral ischemia, Guillain-Barré Syndrome, chronic inflammatory demyelinating polyneuropathy, multiple sclerosis, a lysosomal storage disease, Fabry's disorder, Gaucher's disorder, Niemann Pick C disease, Tay-Sachs disease, and Mucolipidosis type IV, neuropathy, Huntington's disease, a psychiatric disorder, ADHD, schizophrenia, a mood disorder, major depressive disorder, depression, bipolar disorder I, or bipolar disorder II.

53. The method of claim 50, wherein the disease is ALS, FTD, Alzheimer's disease, Parkinson's disease, Huntington's disease, or CMT.

54. The method of claim 50, wherein the disease is ALS.

55. The method of claim 50, wherein the disease is a tauopathy such as Alzheimer's disease, progressive supranuclear palsy, corticobasal syndrome, frontotemporal dementia, or chronic traumatic encephalopathy.

56. The method of claim 50, wherein the disease is a psychiatric disorder such as ADHD, schizophrenia, or mood disorders such as major depressive disorder, depression, bipolar disorder I, or bipolar disorder II.

57. The method of claim 50, wherein the disease is associated with PI3K activity.

58. The method of claim 57, wherein the PI3K is PI3Kα, PI3Kβ, PI3Kδ, and/or PI3Kγ.

59. A compound of any one of claims 1 to 47 for use as a medicament.

60. The compound of claim 59, wherein the compound is for use in treating a neurological disease treatable by inhibition of PIKfyve and/or a PI3 kinase.

61. Use of a compound of any one of claims 1 to 47 in the manufacture of a medicament for treating a neurological disease in a subject in which PIKfyve or PI3K contributes to the pathology and/or symptoms of the neurological disease.
